(19) United States
(12) Patent Application Publication
(10) Pub. No.: US 2025/0157636 A1
(43) Pub. Date: May 15, 2025

(54) GRAPHICAL USER INTERFACE FOR DISCOVERING EFFICIENCY INFORMATION FOR SURGICAL AND HOSPITAL PROCESSES

(71) Applicant: Intuitive Surgical Operations, Inc., Sunnyvale, CA (US)

(72) Inventors: Reza Khodayi Mehr, Sunnyvale, CA (US); Omid Mohareri, San Francisco, CA (US)

(73) Assignee: Intuitive Surgical Operations, Inc., Sunnyvale, CA (US)

(21) Appl. No.: 18/942,106

(22) Filed: Nov. 8, 2024

Related U.S. Application Data
(60) Provisional application No. 63/597,599, filed on Nov. 9, 2023.

(51) Int. Cl. G16H 40/20; G06F 3/0482

(52) U.S. Cl. CPC G16H 40/20; G06F 3/0482

(57) ABSTRACT

Data streams of information of medical procedures are received. The information includes case metadata of the medical procedures, a timeline of phases and tasks within each phase determined for each medical procedure, and three-dimensional point cloud data for each medical procedure during at least portions of phases and tasks within each phase. At least a portion of the information is provided for display using a hierarchical user interface structure. The hierarchical user interface structure includes a first level of a user interface to display, based at least on the three-dimensional point cloud data, a three-dimensional point cloud representation of a task of a phase selected from a timeline of a second level of user interface. The hierarchical structure includes the second level of the user interface to display the timeline and a portion of the case metadata associated with the timeline.

### CROSS-REFERENCE TO RELATED APPLICATION

This application claims the benefit of, and priority to, U.S. Patent Application No. 63/597,599, filed Nov. 9, 2023, the full disclosure of which is incorporated herein in its entirety.

### TECHNICAL FIELD

Various of the disclosed embodiments relate to systems, apparatuses, methods, and non-transitory computer-readable media for providing graphical user interfaces for discovery efficiency information for surgical and hospital processes.

### BACKGROUND

Surgical theaters present unique challenges and operating conditions, which require team members to quickly and efficiently adapt to a variety of rapidly changing technologies and circumstances. Failure of one or more team members to perform efficiently under these challenging conditions may precipitate errors and inefficiencies, which may cause a cascade of downstream delays, imposing undesirable costs and potential risks to patient health. Neither is the potential for such direct and indirect harm limited to inefficiencies occurring during the surgical operations themselves, but also actions taken before, between, and after such procedures. During these “nonoperative” periods, team members must reset the theater from past surgeries and configure the theater for upcoming procedures. Though frequently overlooked, poor performance during these nonoperative periods can itself result in a variety of costly, and potentially harmful, downstream adverse events. Failure to timely sort and store equipment, dilatory transport of patients to and from the theater, excessive and unnecessary motion when working near the patient, and similar nonoperative period inefficiencies can directly cause harm to the patient, impose costs, or may indirectly invite harm or costs via their effects upon downstream tasks.

Unfortunately, identifying, let alone remediating, such nonoperative inefficiencies can be so difficult as to often be intractable. Team members are themselves so focused upon patient care and the performance of their current tasks that opportunities to reflect upon their actions, to recognize the consequences of their actions upon other team members and downstream activities, and to appreciate inefficiencies in the team's collective dynamic as whole, rarely, if ever, occur. While third parties may observe the team's performance within the theater, or subsequently via in-theater sensor data, these observers are themselves subject to reviewer fatigue, reviewer subjectivity, and their presence imposes an undesirable temporal and financial constraint upon performance assessment. Indeed, such reviewers must be expertly trained and can review only a small number of procedures at a time. Furthermore, such human-in-the-loop coaching complicates verification that team members have adhered to any provided feedback, as there is regular turnover among reviewers and team members regularly transition between teams monitored by different reviewers.

While automated data acquisition via in-theater sensors may increase the number of nonoperative periods that can be reviewed, nonoperative periods present unique data management difficulties, as the same data or type of data available during operative periods may no longer be available. For example, reviewers may be able to review handheld instrument kinematics data or robotic surgical system events data captured during a surgery, whereas theses datasets will be unavailable during nonoperative periods (when such equipment is typically inactive or absent). As inefficiencies in the nonoperative preparation may precipitate changes in the downstream operating period instrument kinematics data, discerning such causal relationships by manual inspection of the disparate datasets in isolation can be very difficult.

Conventionally, graphical user interfaces (GUIs) provided for hospitals, hospital consultants, students, and operating rooms (ORs) rely on system events and logs of hospital procedures, rendering such GUIs unreliable, inaccurate, and outdated. Conventional GUIs are also overly complicated and require steep learning curves, given that those GUIs do not provide fast discovery of relevant information and are instead passive tools.

### BRIEF DESCRIPTION OF THE DRAWINGS

Various of the embodiments introduced herein may be better understood by referring to the following Detailed Description in conjunction with the accompanying drawings, in which like reference numerals indicate identical or functionally similar elements:

FIG. 1A is a schematic view of various elements appearing in a surgical theater during a surgical operation, as may occur in relation to some embodiments;

FIG. 1B is a schematic view of various elements appearing in a surgical theater during a surgical operation employing a robotic surgical system, as may occur in relation to some embodiments;

FIG. 2A is a schematic depth map rendering from an example theater-wide sensor perspective, as may be used in some embodiments;

FIG. 2B is a schematic top-down view of objects in the theater of FIG. 2A, with corresponding sensor locations;

FIG. 2C is a pair of images depicting a grid-like pattern of orthogonal rows and columns in perspective, as captured from a theater-wide visual image sensor having a rectilinear view and a theater-wide visual image sensor having a fisheye view, each of which may be used in connection with some embodiments;

FIG. 3 is a schematic representation of a series of surgical procedures within a surgical theater, their intervening nonoperative periods, and corresponding theater-wide sensor datasets for one such nonoperative period, as may occur in connection with some embodiments;

FIG. 4 is a schematic block diagram illustrating an example deployment topology for a nonoperative periods analysis system, as may be implemented in some embodiments;

FIG. 5A is a schematic representation of a collection of metrics intervals, as may be used to assess nonoperative team performance in some embodiments;

FIG. 5B is a schematic processing diagram indicating full-day relations of various intervals, including intervals from FIG. 5A, as may be applied in some embodiments;

FIG. 5C is a schematic block diagram indicating possible activity analysis class groupings, as may be used in connection with some embodiments;

FIG. 6 is a table of example task action temporal definitions, as may be used in some embodiments;

FIG. 7 is a table of additional example task action temporal definitions, as may be used in some embodiments;

FIG. 8 is a schematic block diagram illustrating various metrics and their relation in constructing a composite score (referred to as an OR analysis “ORA” score), as may be used in some embodiments;

FIG. 9A is a schematic block diagram depicting a general nonoperative analysis system processing flow, as may be implemented in some embodiments;

FIG. 9B is a schematic block diagram depicting elements in a more detailed example nonoperative analysis system processing flow than the flow depicted in FIG. 9A, as may be implemented in some embodiments;

FIG. 9C is a flow diagram illustrating various operations in an example overall process for analyzing theater-wide sensor data during nonoperative periods, as may be implemented in some embodiments;

FIG. 10 is a flow diagram illustrating various operations in an example nonoperative segment detection process, as may be performed in some embodiments;

FIG. 11A is a schematic block diagram illustrating an example information processing flow for performing object detection, as may be used in connection with some embodiments;

FIG. 11B is a flow diagram illustrating various operations in an example process for performing object detection, as may be used in some embodiments;

FIG. 12A is schematic block diagram illustrating an example object tracking information processing flow, as may be used in connection with some embodiments;

FIG. 12B is flow diagram illustrating various operations in an example process for performing object tracking, as may be used in connection with some embodiments;

FIG. 13A is a schematic visual image and depth frame theater-wide data pair, from theater-wide data video, with an indication of the optical-flow derived correspondence, as may be used in some embodiments;

FIG. 13B is a schematic top-down view of the scene depicted in FIG. 13A;

FIG. 13C is a schematic pair of visual images showing team member motion distant from and near to an imaging sensor;

FIG. 13D is a schematic top-down view depicting the team member motion presented in the visual images of FIG. 13C;

FIG. 14 is a flow diagram illustrating various operations in an example process for performing motion analysis of nonoperative periods from theater-wide data, as may be used in connection with some embodiments;

FIG. 15 is flow diagram illustrating various operations in an example process for performing clustering and outlier determination analysis based upon metric values, such as those disclosed herein, as may be performed in some embodiments;

FIG. 16 is flow diagram illustrating various operations in an example process for providing coaching feedback based upon determined metric values, as may be performed in some embodiments;

FIG. 17 is a schematic representation of GUI elements in an example dashboard interface layout for nonoperative metrics quick review, as may be implemented in some embodiments;

FIG. 18A is a schematic representation of a GUI element in an example global nonoperative metrics quick review dashboard, as may be implemented in some embodiments;

FIG. 18B is a schematic representation of arrow graphical elements, as may be used in, e.g., the element of FIG. 18A in some embodiments;

FIG. 18C is a schematic representation of an example global nonoperative metrics quick review dashboard layout, as may be implemented in some embodiments;

FIG. 19A is a plot of example interval metric values acquired in connection with an example prototype implementation of an embodiment;

FIG. 19B is a plot of example interval metric values as acquired in connection with an example prototype implementation of an embodiment;

FIG. 20A is a plot of example interval metric values as acquired in connection with an example prototype implementation of an embodiment;

FIG. 20B is a plot of example interval metric values as acquired in connection with an example prototype implementation of an embodiment;

FIG. 21A is a plot of example metric values as acquired in connection with an example prototype implementation of an embodiment;

FIG. 21B is a plot of example metric values as acquired in connection with an example prototype implementation of an embodiment;

FIG. 21C is a plot of example metric values as acquired in connection with an example prototype implementation of an embodiment;

FIG. 22 is a schematic representation of example GUI elements for providing metrics-derived feedback, as may be used in some embodiments;

FIG. 23 is an example schematic data processing overview diagram corresponding to aspects of FIG. 4, as may be used in connection with some embodiments;

FIG. 24 is a screenshot of a feedback interface corresponding to aspects of FIG. 22, as may be used in connection with some embodiments;

FIG. 25 is a screenshot of a feedback interface corresponding to aspects of FIG. 22, as may be used in connection with some embodiments;

FIG. 26 is a screenshot of a feedback interface corresponding to aspects of FIG. 17, as may be used in connection with some embodiments;

FIG. 27 is a collection of color image plots for example metric values corresponding to aspects of FIGS. 21A-C, as acquired in connection with an example prototype implementation of an embodiment;

FIG. 28 is a collection of color plots corresponding to aspects of the plots of FIGS. 19A-B and 20A-B;

FIG. 29A is a collection of photographs of theater-wide sensor visual images captured in a surgical theater during various tasks;

FIG. 29B is a visual image and a depth frame each captured with a theater-wide sensor and related photographs of an example theater-wide sensor data capture platform, as may be used in some embodiments;

FIG. 30 is a block diagram of an example computer system as may be used in conjunction with some of the embodiments;

FIG. 31 is an example schematic diagram illustrating an example hierarchical structure for displaying information of a plurality of medical procedures, according to some embodiments;

FIG. 32A is a screenshot illustrating an example user interface showing highlights of the information of a plurality of medical procedures, according to some examples.

FIG. 32B is a screenshot illustrating an example user interface showing highlights of the information of a plurality of medical procedures, according to some examples.

FIG. 33 is a screenshot illustrating an example user interface showing aggregated data of the information of a plurality of medical procedures, according to some examples.

FIG. 34 is a screenshot illustrating an example user interface showing aggregated data of the information of a plurality of medical procedures, according to some examples.

FIG. 35 is a screenshot illustrating an example user interface showing aggregated data of the information of a plurality of medical procedures, according to some examples.

FIG. 36 is a screenshot illustrating an example user interface showing individual case data of the information of a plurality of medical procedures, according to some examples.

FIG. 37 is a screenshot illustrating an example user interface showing timelines of the information of a plurality of medical procedures, according to some examples.

FIG. 38 is a screenshot illustrating an example user interface showing interactive filter, according to some examples.

FIG. 39 is a screenshot illustrating an example user interface showing interactive filter, according to some examples.

FIG. 40 is a screenshot illustrating an example user interface showing suggested view element, according to some examples.

FIG. 41 is a screenshot illustrating an example user interface showing nonoperative metrics for multiple surgeons and multiple phases/tasks, according to some examples.

FIG. 42 is a screenshot illustrating an example user interface showing nonoperative metrics for multiple surgeons for a given procedure (inguinal hernia repair), according to some examples.

FIG. 43 is a schematic block diagram depicting a method for providing medical information of a plurality of medical procedures using a hierarchical structure, as may be implemented in some embodiments.

FIG. 44 is a schematic block diagram depicting a method for providing medical information of a plurality of medical procedures using a hierarchical structure, as may be implemented in some embodiments.

The specific examples depicted in the drawings have been selected to facilitate understanding. Consequently, the disclosed embodiments should not be restricted to the specific details in the drawings or the corresponding disclosure. For example, the drawings may not be drawn to scale, the dimensions of some elements in the figures may have been adjusted to facilitate understanding, and the operations of the embodiments associated with the flow diagrams may encompass additional, alternative, or fewer operations than those depicted here. Thus, some components and/or operations may be separated into different blocks or combined into a single block in a manner other than as depicted. The embodiments are intended to cover all modifications, equivalents, and alternatives falling within the scope of the disclosed examples, rather than limit the embodiments to the particular examples described or depicted.

### DETAILED DESCRIPTION

Accordingly, there exists a need for systems and methods to overcome challenges and difficulties such as those described above. For example, there exists a need for systems and methods to process disparate forms of surgical theater data acquired during nonoperative periods so as to facilitate reviewer analysis and feedback generation based upon team member inefficiencies identified therein.

### Example Surgical Theaters Overview

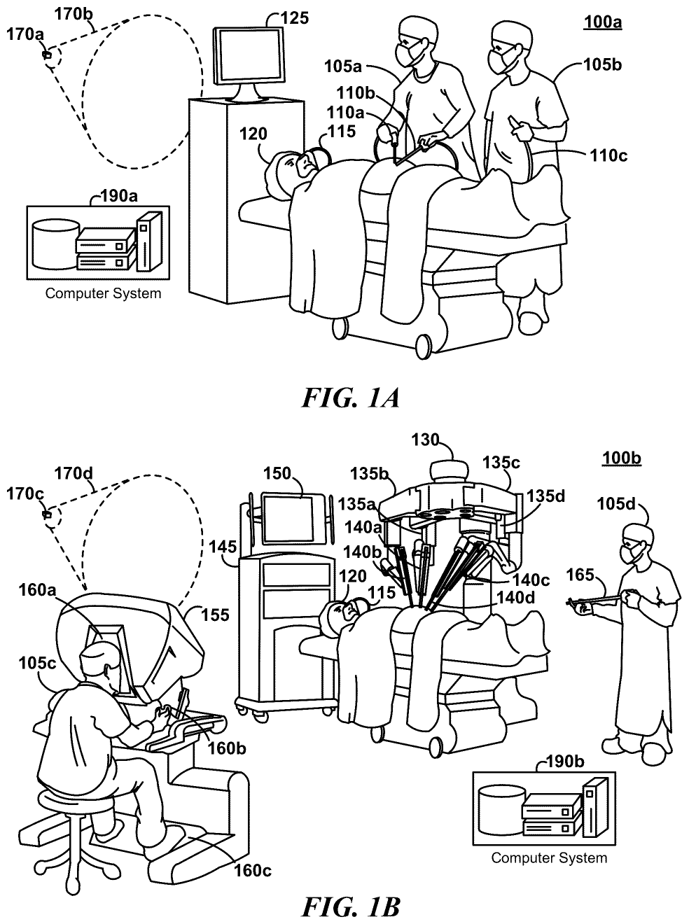

FIG. 1A is a schematic view of various elements appearing in a surgical theater 100a during a surgical operation as may occur in relation to some embodiments. Particularly, FIG. 1A depicts a non-robotic surgical theater 100a, wherein a patient-side surgeon 105a performs an operation upon a patient 120 with the assistance of one or more assisting members 105b, who may themselves be surgeons, physician's assistants, nurses, technicians, etc. The surgeon 105a may perform the operation using a variety of tools, e.g., a visualization tool 110b such as a laparoscopic ultrasound, visual image acquiring endoscope, etc., and a mechanical instrument 110a such as scissors, retractors, a dissector, etc.

The visualization tool 110b provides the surgeon 105a with an interior view of the patient 120, e.g., by displaying visualization output from an imaging device mechanically and electrically coupled with the visualization tool 110b. The surgeon may view the visualization output, e.g., through an eyepiece coupled with visualization tool 110b or upon a display 125 configured to receive the visualization output. For example, where the visualization tool 110b is a visual image acquiring endoscope, the visualization output may be a color or grayscale image. Display 125 may allow assisting member 105b to monitor surgeon 105a's progress during the surgery. The visualization output from visualization tool 110b may be recorded and stored for future review, e.g., using hardware or software on the visualization tool 110b itself, capturing the visualization output in parallel as it is provided to display 125, or capturing the output from display 125 once it appears on-screen, etc. While two-dimensional video capture with visualization tool 110b may be discussed extensively herein, as when visualization tool 110b is a visual image endoscope, one will appreciate that, in some embodiments, visualization tool 110b may capture depth data instead of, or in addition to, two-dimensional image data (e.g., with a laser rangefinder, stereoscopy, etc.).

A single surgery may include the performance of several groups (e.g., phases or stages) of actions, each group of actions forming a discrete unit referred to herein as a task. For example, locating a tumor may constitute a first task, excising the tumor a second task, and closing the surgery site a third task. Each task may include multiple actions, e.g., a tumor excision task may require several cutting actions and several cauterization actions. While some surgeries require that tasks assume a specific order (e.g., excision occurs before closure), the order and presence of some tasks in some surgeries may be allowed to vary (e.g., the elimination of a precautionary task or a reordering of excision tasks where the order has no effect). Transitioning between tasks may require the surgeon 105a to remove tools from the patient, replace tools with different tools, or introduce new tools. Some tasks may require that the visualization tool 110b be removed and repositioned relative to its position in a previous task. While some assisting members 105b may assist with surgery-related tasks, such as administering anesthesia 115 to the patient 120, assisting members 105b may also assist with these task transitions, e.g., anticipating the need for a new tool 110c.

Advances in technology have enabled procedures such as that depicted in FIG. 1A to also be performed with robotic systems, as well as the performance of procedures unable to be performed in non-robotic surgical theater 100a. Specifically, FIG. 1B is a schematic view of various elements appearing in a surgical theater 100b during a surgical operation employing a robotic surgical system, such as a da Vinci™ surgical system, as may occur in relation to some embodiments. Here, patient side cart 130 having tools 140a, 140b, 140c, and 140d attached to each of a plurality of arms 135a, 135b, 135c, and 135d, respectively, may take the position of patient-side surgeon 105a. As before, one or more of tools 140a, 140b, 140c, and 140d may include a visualization tool (here visualization tool 140d), such as a visual image endoscope, laparoscopic ultrasound, etc. An operator 105c, who may be a surgeon, may view the output of visualization tool 140d through a display 160a upon a surgeon console 155. By manipulating a hand-held input mechanism 160b and pedals 160c, the operator 105c may remotely communicate with tools 140a-d on patient side cart 130 so as to perform the surgical procedure on patient 120. Indeed, the operator 105c may or may not be in the same physical location as patient side cart 130 and patient 120 since the communication between surgeon console 155 and patient side cart 130 may occur across a telecommunication network in some embodiments. An electronics/control console 145 may also include a display 150 depicting patient vitals and/or the output of visualization tool 140d.

Similar to the task transitions of non-robotic surgical theater 100a, the surgical operation of theater 100b may require that tools 140a-d, including the visualization tool 140d, be removed or replaced for various tasks as well as new tools, e.g., new tool 165, be introduced. As before, one or more assisting members 105d may now anticipate such changes, working with operator 105c to make any necessary adjustments as the surgery progresses.

Also similar to the non-robotic surgical theater 100a, the output from the visualization tool 140d may here be recorded, e.g., at patient side cart 130, surgeon console 155, from display 150, etc. While some tools 110a, 110b, 110c in non-robotic surgical theater 100a may record additional data, such as temperature, motion, conductivity, energy levels, etc., the presence of surgeon console 155 and patient side cart 130 in theater 100b may facilitate the recordation of considerably more data than is only output from the visualization tool 140d. For example, operator 105c's manipulation of hand-held input mechanism 160b, activation of pedals 160c, eye movement with respect to display 160a, etc., may all be recorded. Similarly, patient side cart 130 may record tool activations (e.g., the application of radiative energy, closing of scissors, etc.), movement of instruments, etc., throughout the surgery. In some embodiments, the data may have been recorded using an in-theater recording device, which may capture and store sensor data locally or at a networked location (e.g., software, firmware, or hardware configured to record surgeon kinematics data, console kinematics data, instrument kinematics data, system events data, patient state data, etc., during the surgery).

Within each of theaters 100a, 100b, or in network communication with the theaters from an external location, may be computer systems 190a and 190b, respectively (in some embodiments, computer system 190b may be integrated with the robotic surgical system, rather than serving as a standalone workstation). As will be discussed in greater detail herein, the computer systems 190a and 190b may facilitate, e.g., data collection, data processing, etc.

Similarly, many of theaters 100a, 100b may include sensors placed around the theater, such as sensors 170a and 170c, respectively, configured to record activity within the surgical theater from the perspectives of their respective fields of view 170b and 170d. Sensors 170a and 170c may be, e.g., visual image sensors (e.g., color or grayscale image sensors), depth-acquiring sensors (e.g., via stereoscopically acquired visual image pairs, via time-of-flight with a laser rangefinder, structural light, etc.), or a multi-modal sensor including a combination of a visual image sensor and a depth-acquiring sensor (e.g., a red green blue depth RGB-D sensor). In some embodiments, sensors 170a and 170c may also include audio acquisition sensors or sensors specifically dedicated to audio acquisition may be placed around the theater. A plurality of such sensors may be placed within theaters 100a, 100b, possibly with overlapping fields of view and sensing range, to achieve a more holistic assessment of the surgery. For example, depth-acquiring sensors may be strategically placed around the theater so that their resulting depth frames at each moment may be consolidated into a single three-dimensional virtual element model depicting objects in the surgical theater. Examples of a three-dimensional virtual element model include a three-dimensional point cloud (also referred to as three-dimensional point cloud data). Similarly, sensors may be strategically placed in the theater to focus upon regions of interest. For example, sensors may be attached to display 125, display 150, or patient side cart 130 with fields of view focusing upon the patient 120's surgical site, attached to the walls or ceiling, etc. Similarly, sensors may be placed upon console 155 to monitor the operator 105c. Sensors may likewise be placed upon movable platforms specifically designed to facilitate orienting of the sensors in various poses within the theater.

As used herein, a “pose” refers to a position or location and an orientation of a body. For example, a pose refers to the translational position and rotational orientation of a body. For example, in a three-dimensional space, one may represent a pose with six total degrees of freedom. One will readily appreciate that poses may be represented using a variety of data structures, e.g., with matrices, with quaternions, with vectors, with combinations thereof, etc. Thus, in some situations, when there is no rotation, a pose may include only a translational component. Conversely, when there is no translation, a pose may include only a rotational component.

Similarly, for clarity, “theater-wide” sensor data refers herein to data acquired from one or more sensors configured to monitor a specific region of the theater (the region encompassing all, or a portion, of the theater) exterior to the patient, to personnel, to equipment, or to any other objects in the theater, such that the sensor can perceive the presence within, or passage through, at least a portion of the region of the patient, personnel, equipment, or other objects, throughout the surgery. Sensors so configured to collect such “theater-wide” data are referred to herein as “theater-wide sensors.” For clarity, one will appreciate that the specific region need not be rigidly fixed throughout the procedure, as, e.g., some sensors may cyclically pan their field of view so as to augment the size of the specific region, even though this may result in temporal lacunae for portions of the region in the sensor's data (lacunae which may be remedied by the coordinated panning or fields of view of other nearby sensors). Similarly, in some cases, personnel or robotics systems may be able to relocate theater-wide sensors, changing the specific region, throughout the procedure, e.g., to better capture different tasks. Accordingly, sensors 170a and 170c are theater-wide sensors configured to produce theater-wide data. “Visualization data” refers herein to visual image or depth image data captured from a sensor. Thus, visualization data may or may not be theater-wide data. For example, visualization data captured at sensors 170a and 170c is theater-wide data, whereas visualization data captured via visualization tool 140d would not be theater-wide data (for at least the reason that the data is not exterior to the patient).

### Example Theater-Wide Sensor Topologies

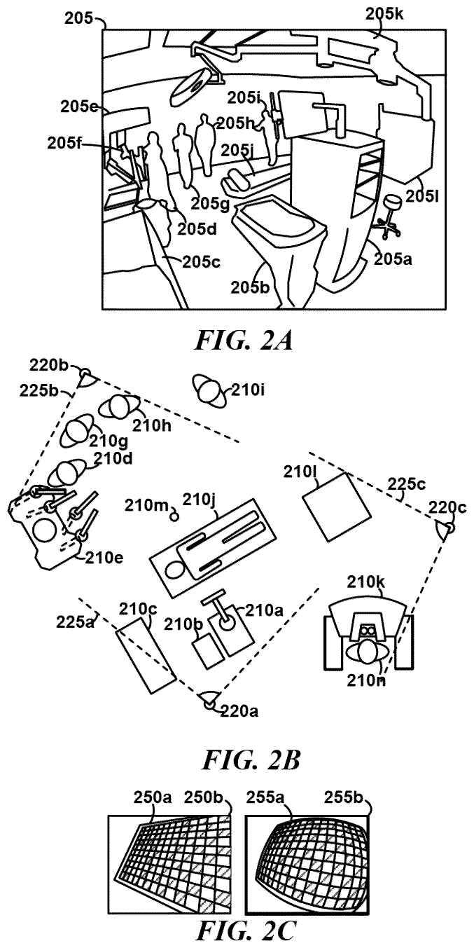

For further clarity regarding theater-wide sensor deployment, FIG. 2A is a schematic depth map rendering from an example theater-wide sensor perspective 205 as may be used in some embodiments. Specifically, this example depicts depth values corresponding to an electronics/control console 205a (e.g., the electronics/control console 145) and a nearby tray 205b, and cabinet 205c. Also within the field of view are depth values associated with a first technician 205d, presently adjusting a robotic arm (associated with depth values 205f) upon a robotic surgical system (associated with depth values 205e). Team members, with corresponding depth values 205g, 205h, and 205i, likewise appear in the field of view, as does a portion of the surgical table 205j. Depth values 205l corresponding to a movable dolly and a boom with a lighting system's depth values 205k also appear within the field of view.

The theater-wide sensor capturing the perspective 205 may be only one of several sensors placed throughout the theater. For example, FIG. 2B is a schematic top-down view of objects in the theater at a given moment during the surgical operation. Specifically, the perspective 205 may have been captured via a theater-wide sensor 220a with corresponding field of view 225a. Thus, for clarity, cabinet depth values 205c may correspond to cabinet 210c, electronics/control console depth values 205a may correspond to electronics/control console 210a, and tray depth values 205b may correspond to tray 210b. Robotic system 210e may correspond to depth values 205e, and each of the individual team members 210d, 210g, 210h, and 210i may correspond to depth values 205d, 205g, 205h, and 205i, respectively. Similarly, dolly 2101 may correspond to depth values 205l. Depth values 205j may correspond to table 210j (with an outline of a patient shown here for clarity, though the patient has not yet been placed upon the table corresponding to depth values 205j in the example perspective 205). A top-down representation of the boom corresponding to depth values 205k is not shown for clarity, though one will appreciate that the boom may likewise be considered in various embodiments.

As indicated, each of the sensors 220a, 220b, 220c is associated with different fields of view 225a, 225b, and 225c, respectively. The fields of view 225a-c may sometimes have complementary characters, providing different perspectives of the same object, or providing a view of an object from one perspective when it is outside, or occluded within, another perspective. Complementarity between the perspectives may be dynamic both spatially and temporally. Such dynamic character may result from movement of an object being tracked, but also from movement of intervening occluding objects (and, in some cases, movement of the sensors themselves). For example, at the moment depicted in FIGS. 2A and 2B, the field of view 225a has only a limited view of the table 210j, as the electronics/control console 210a substantially occludes that portion of the field of view 225a. Consequently, in the depicted moment, the field of view 225b is better able to view the surgical table 210j. However, neither field of view 225b nor 225a has an adequate view of the operator 210n in console 210k. To observe the operator 210n (e.g., when they remove their head in accordance with “head out” events), field of view 225c may be more suitable. However, over the course of the data capture, these complementary relationships may change. For example, before the procedure begins, electronics/control console 210a may be removed and the robotic system 210e moved into the position 210m. In this configuration, field of view 225a may instead be much better suited for viewing the patient table 210j than the field of view 225b. As another example, movement of the console 210k to the presently depicted pose of electronics/control console 210a may render field of view 225a more suitable for viewing operator 210n, than field of view 225c. Suitability of a field of view may thus depend upon the number and duration of occlusions, quality of the field of view (e.g., how close the object of interest is to the sensor), and movement of the object of interest within the theater. Such changes may be transitory and short in duration, as when a team member moving in the theater briefly occludes a sensor, or they may be chronic or sustained, as when equipment is moved into a fixed position throughout the duration of the procedure.

As mentioned, the theater-wide sensors may take a variety of forms and may, e.g., be configured to acquire visual image data, depth data, both visual and depth data, etc. One will appreciate that visual and depth image captures may likewise take on a variety of forms, e.g., to afford increased visibility of different portions of the theater. For example, FIG. 2C is a pair of images 250b, 255b depicting a grid-like pattern of orthogonal rows and columns in perspective, as captured from a theater-wide sensor having a rectilinear view and a theater-wide sensor having a fisheye view, respectively. More specifically, some theater-wide sensors may capture rectilinear visual images or rectilinear depth frames, e.g., via appropriate lenses, post-processing, combinations of lenses and post-processing, etc. while other theater-wide sensors may instead, e.g., acquire fisheye or distorted visual images or rectilinear depth frames, via appropriate lenses, post-processing, combinations of lenses and post-processing, etc. For clarity, image 250b depicts a checkboard pattern in perspective from a rectilinear theater wide sensor. Accordingly, the orthogonal rows and columns 250a shown here in perspective, retain linear relations with their vanishing points. In contrast, image 255b depicts the same checkboard pattern in the same perspective, but from a fish-eye theater-wise sensor perspective. Accordingly, the orthogonal rows and columns 255a, while in reality retaining a linear relationship with their vanishing points (as they appear in image 250b) appear here from the sensor data as having curved relations with their vanishing points. Thus, each type of sensor, and other sensor types, may be used alone, or in some instances, in combination, in connection with various embodiments.

Similarly, one will appreciate that not all sensors may acquire perfectly rectilinear, fisheye, or other desired mappings. Accordingly, checkered patterns, or other calibration fiducials (such as known shapes for depth systems), may facilitate determination of a given theater-wide sensor's intrinsic parameters. For example, the focal point of the fisheye lens, and other details of the theater-wide sensor (principal points, distortion coefficients, etc.), may vary between devices and even across the same device over time. Thus, it may be necessary to recalibrate various processing methods for the particular device at issue, anticipating the device variation when training and configuring a system for machine learning tasks. Additionally, one will appreciate that the rectilinear view may be achieved by undistorting the fisheye view once the intrinsic parameters of the camera are known (which may be useful, e.g., to normalize disparate sensor systems to a similar form recognized by a machine learning architecture). Thus, while a fisheye view may allow the system and users to more readily perceive a wider field of view than in the case of the rectilinear perspective, when a processing system is considering data from some sensors acquiring undistorted perspectives and other sensors acquiring distorted perspectives, the differing perspectives may be normalized to a common perspective form (e.g., mapping all the rectilinear data to a fisheye representation or vice versa).

### Example Surgical Theater Nonoperative Data

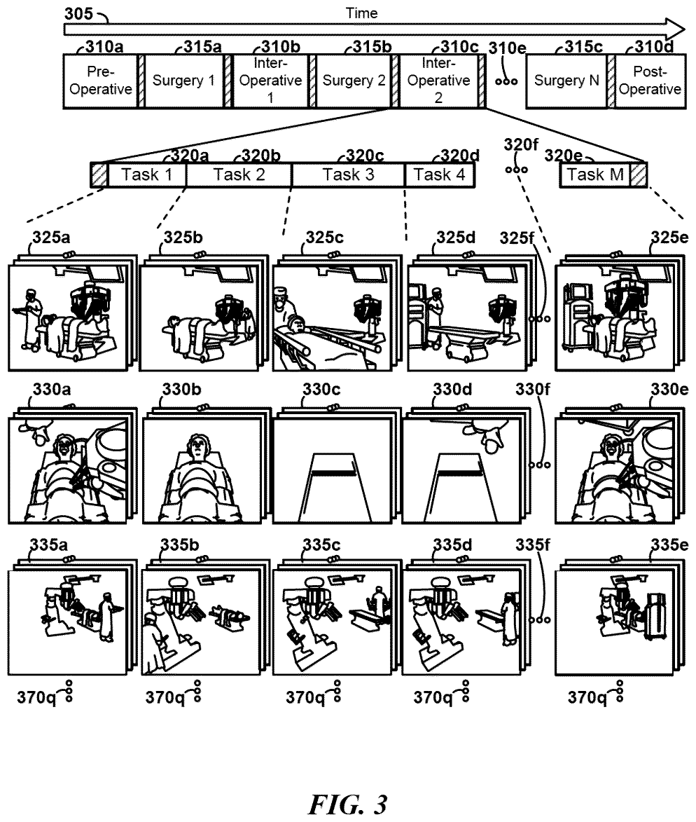

As discussed above, granular and meaningful assessment of team member actions and performance during nonoperative periods in a theater may reveal opportunities to improve efficiency and to avoid inefficient behavior having the potential to affect downstream operative and nonoperative periods. For context, FIG. 3 depicts a state of a single operating room over time 305, e.g., over the course of a day. In this example, during an initial pre-surgical period 310a, the team may prepare the operating room for the day's procedures, collecting appropriate equipment, reviewing scheduled tasks, etc. After performing the day's first surgery 315a, a nonoperative inter-operative period 310b will follow wherein the team performs the turnover from the operating room configuration for performing the surgery 315a to the configuration for performing the surgery 315b. Such alternating nonoperative and operative periods may continue throughout the day, e.g., nonoperative inter-surgical period 310c here follows the second surgery 315b, etc. After the final procedure 315c is performed for the day, the team may perform any final maintenance operations, may secure and put away equipment, deactivate devices, upload data, etc., during the post-operative period 310d. Ellipsis 310e indicates the possibility of additional intervening operative and nonoperative states (though, naturally, in some theaters there may instead by only one surgery during the day). Because of the theater operations' sequential character, an error in an upstream period can cause errors and delays to cascade through downstream periods. For example, improper alignment of equipment during pre-surgical period 310a may result in a delay during surgery 315a. This delay may itself require nonoperative period 310b to be shortened, providing a team member insufficient time to perform proper cleaning procedures, thereby placing the patient of surgery 315b's health at risk. Thus, inefficiencies early in the day may result in the delay, poor execution, or rescheduling of downstream actions. Conversely, efficiencies early in the day may provide tolerance downstream for unexpected events, facilitating more predictable operation outcomes and other benefits.

Each of the theater states, including both the operative periods 315a, 315b, etc. and nonoperative periods 310a, 310b, 310c, 310d, etc. may be divided into a collection of tasks. For example, the nonoperative period 310c may be divided into the tasks 320a, 320b, 320c, 320d, and 320e (with intervening tasks represented by ellipsis 320f). In this example, at least three theater-wide sensors were present in the OR, each sensor capturing at least visual image data (though one will appreciate that there may be fewer than three streams, or more, as indicated by ellipses 370q). Specifically, a first theater-wide sensor captured a collection of visual images 325a (e.g., visual image video) during the first nonoperative task 320a, a collection of visual images 325b during the second nonoperative task 320b, a collection of visual images 325c during the third nonoperative task 320c, a collection of visual images 325d during the fourth nonoperative task 320d, and the collection of visual images 325e during the last nonoperative task 320e (again, intervening groups of frames may have been acquired for other tasks as indicated by ellipsis 325f).

Contemporaneously during each of the tasks of the second nonoperative period 310c, the second theater-wide sensor may acquire the data collections 330a-e (ellipsis 330f depicting possible intervening collections), and the third theater-wide sensor may acquire the collections of 335a-e (ellipsis 335f depicting possible intervening collections). Thus, one will appreciate, e.g., that the data in sets 325a, 330a, and 335a may be acquired contemporaneously by the three theater-wide sensors during the task 320a (and, similarly, each of the other columns of collected data associated with each respective nonoperative task). Again, though visual images are shown in this example, one will appreciate that other data, such as depth frames, may alternatively, or additionally, be likewise acquired in each collection.

Thus, in task 320a, which may be an initial “cleaning” task following the surgery 315b, the sensor associated with collections 325a-e depicts a team member and the patient in a first perceptive. In contrast, the sensor capturing collections 335a-e is located on the opposite side of the theater and provides a fisheye view from a different perspective. Consequently, the second sensor's perception of the patient is more limited. The sensor associated with collections 330a-e is focused upon the patient, however, this sensor's perspective doesn't depict the team member very well in the collection 330a, whereas the collection 325a does provide a clear view of the team member.

Similarly, in task 320b, which may be a “roll-back” task, moving the robotic system away from the patient, the theater-wide sensor associated with collections 330a-e depicts that the patient is no longer subject to anesthesia, but does not depict the state of the team member relocating the robotic system. Rather, the collections 325b and 335b each depict the team member and the new pose of the robotic system at a point distant from the patient and operating table (though the sensor associated with the stream collections 335a-e is better positioned to observe the robot in its post-rollback pose).

In task 320c, which may be a “turnover” or “patient out” task, a team member escorts the patient out of the operating room. While the theater-wide sensor associated with collection 325c has a clear view of the departing patient, the theater-wide sensor associated with the collection 335c may be too far away to observe the departure in detail. Similarly, the collection 330c only indicates that the patient is no longer on the operating table.

In task 320d, which may be a “setup” task, a team member positions equipment which will be used in the next operative period (e.g., the final surgery 315c if there are no intervening periods in the ellipsis 310e).

Finally, in task 320e, which may be a “sterile prep” task before the initial port placements and beginning of the next surgery (again, e.g., surgery 315c), the theater-wide sensor associated with collection 330e is able to perceive the pose of the robotic system and its arms, as well as the state of the new patient. Conversely, collections 325e and 335e may provide wider contextual information regarding the state of the theater.

Thus, one can appreciate the holistic benefit of multiple sensor perspectives, as the combined views of the streams 325a-e, 330a-e, and 335a-e may provide overlapping situational awareness. Again, as mentioned, not all of the sensors may acquire data in exactly the same manner. For example the sensor associated with collections 335a-e may acquire data from a fisheye perspective, whereas the sensors associated with collections 325a-e and 330a-e may acquire rectilinear data. Similarly, there may be fewer or more theater-wide sensors and streams than are depicted here. Generally, because each collection is timestamped, it will be possible for a reviewing system to correlate respective streams' representations, even when they are of disparate forms. Thus, data directed to different theater regions may be reconciled and reviewed. Unfortunately, as mentioned, unlike periods 315a-c, surgical instruments, robotic systems, etc., may no longer be capturing data during the nonoperative periods (e.g., periods 310a-d). Accordingly, systems and reviewers regularly accustomed to analyzing the copious datasets available from periods 315a-c may find it especially difficult to review the more sparse data of periods 310a-d as they may need to rely only upon the disparate theater-wide streams 325a-e, 330a-e, and 335a-e. Even as the reader may have perceived in considering this figure, manually reconciling disparate, but contemporaneously captured perspectives, may be cognitively taxing upon a human reviewer.

### Example Nonoperative Activity Data Processing Overview

Various embodiments employ a processing pipeline facilitating analysis of nonoperative periods, and may include methods to facilitate iterative improvement of the surgical team's performance during these periods. Particularly, some embodiments include computer systems configured to automatically measure and analyze nonoperative activities in surgical operating rooms and recommend customized actionable feedback to operating room staff or hospital management based upon historical dataset patterns so as, e.g., to improve workflow efficiency. Such systems can also help hospital management assess the impact of new personnel, equipment, facilities, etc., as well as scale their review to a larger number, and more disparate types, of surgical theaters and surgeries, consequently driving down workflow variability. As discussed, various embodiments may be applied to surgical theaters having more than one modality, e.g., robotic, non-robotic laparoscopic, non-robotic open. Neither are various of the disclosed approaches limited to nonoperative periods associated with specific types of surgical procedures (e.g., prostatectomy, cholecystectomy, etc.).

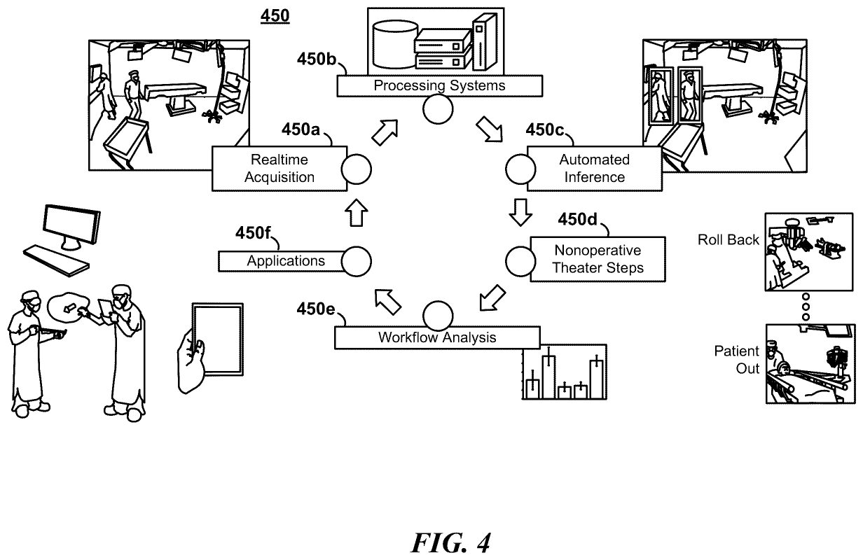

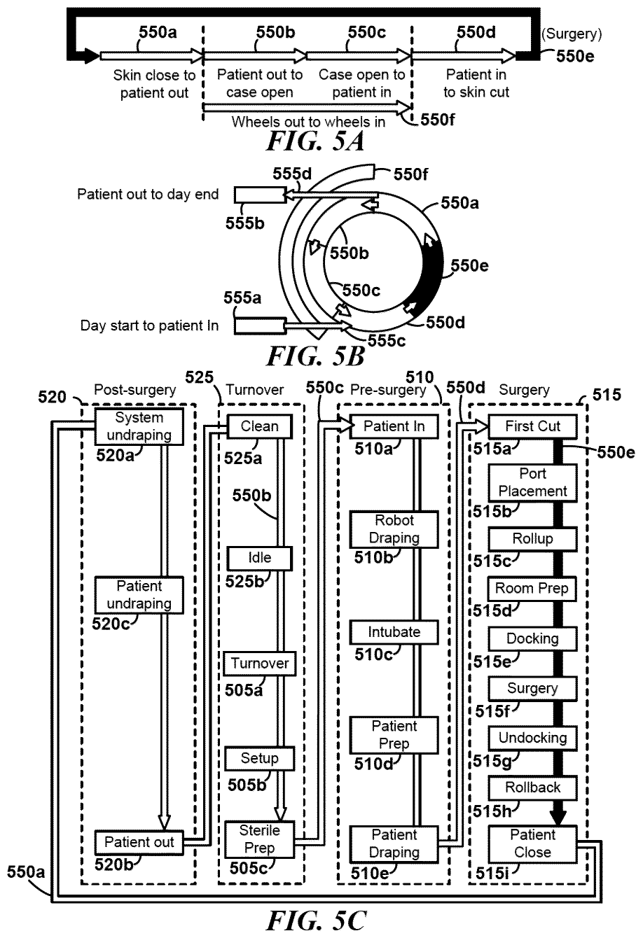

FIG. 4 is a schematic block diagram illustrating an example deployment topology 450 for a nonoperative periods analysis system of certain embodiments. As described herein, during realtime acquisition 450a, data may be collected from one or more theater-wide sensors in one or more perspectives. Multimodal (e.g., visual image and depth) sensor suites within a surgical theater (whether robotic or non-robotic) produce a wide variety of data. Consolidating this data into elemental and composite OR metrics, as described herein, may more readily facilitate analysis. To determine these metrics, the data may be provided to a processing systems 450b, described in greater detail herein, to perform automated inference 450c, including the detection of objects in the theater, such as personnel and equipment, as well as to segment the theater-wide data into distinct steps 450d (which may, e.g., correspond to the groupings and their respective actions discussed herein with respect to FIGS. 5A-C). The discretization of the theater-wide data into the steps 450d may facilitate more meaningful and granular determinations of metrics from the theater-wide data via various workflow analytics 450e, e.g., to ascertain surgical theater efficiency, to provide actionable coaching recommendations, etc.

Following the generation of such metrics during workflow analysis 450e, embodiments also disclose software and algorithms for presentation of the metric values along with other suitable information to users (e.g., consultants, students, medical staff, and so on) and for outlier detection within the metric values relative to historical patterns. As used herein, information of a plurality of medical procedures (e.g., procedure-related information, case-related information, information related to medical environments such as the ORs, and so on) refers to metric values and other associated information determined in the manners described herein. These analytics results may then be used to provide coaching and feedback via various applications 450f. Software applications 450f may present various metrics and derived analysis disclosed herein in various interfaces as part of the actionable feedback, a more rigorous and comprehensive solution than the prior use of human reviewers alone. One will appreciate that such applications 450f may be provided upon any suitable computer system, including desktop applications, tablets, augmented reality devices, etc. Such computer system can be located remote from the surgical theaters 100a and 100b in some examples. In other examples, such computer system can be located within the surgical theaters 100a and 100b (e.g., within the OR or the medical facility in which the hospital or OR processes occur). In one example, a consultant can review the information of a plurality of medical procedures via the applications 450f to provide feedback. In another example, a student can review the information of a plurality of medical procedures via the applications 450f to improve learning experience and to provide feedback. This feedback may result in the adjustment of the theater operation such that subsequent application of the steps 450a-f identify new or more subtle inefficiencies in the team's workflow. Thus, the cycle may continue again, such that the iterative, automated OR workflow analytics facilitate gradual improvement in the team's performance, allowing the team to adapt contextually based on upon the respective adjustments. Such iterative application may also help reviewers to better track the impact of the feedback to the team, analyze the effect of changes to the theater composition and scheduling, as well as for the system to consider historical patterns in future assessments and metrics generation.

### Example Nonoperative Interval Divisions

FIG. 5A is a schematic representation of a collection of metrics intervals as may be used to assess nonoperative team performance in some embodiments. One will appreciate that the intervals may be applied cyclically in accordance with the alternating character of the operative and nonoperative periods in the theater described above in FIG. 3. For example, initially, the surgical operation 315b may correspond to the interval 550e. Following the operation 315b's completion, actions and corresponding data in the theater may be allocated to consecutive intervals 550a-d during the subsequent nonoperative period 310c. Data and actions in the next surgery (e.g., surgery 315c, if there are no intervening periods in ellipsis 310e), may then be ascribed again to a second instance of the interval 550e, and so forth (consequently, data from each of the nonoperative periods 310b, 310b will be allocated to instances of intervals 550a-d). Intervals may also be grouped into larger intervals, as is the case here with the “wheels out to wheels in” interval 550f, which groups the intervals 550b and 550c, sharing the start time of interval 550b and the end time of interval 550c. Consolidating theater-wide data into this taxonomy, in conjunction with various other operations disclosed herein, may more readily facilitate analysis in a manner amenable to larger efficiency review, as described in greater detail herein. For example, organizing data in this manner may facilitate comparisons with different days of the week over the course of the month across theaters, surgery configurations (both robotic and non-robotic), and teams, with specific emphasis upon particular of these intervals 550a-d appearing in the corresponding nonoperative periods. Though not part of the nonoperative period, in some embodiments, it may still be useful to determine the duration of the surgery in interval 550e, as the duration may inform the efficiency or inefficiency of the preceding or succeeding nonoperative period. Accordingly, in some embodiments, some of the disclosed metrics may consider events and actions in this interval 550e, even when seeking ultimately to assess the efficiency of a nonoperative period.

For further clarity in the reader's understanding, FIG. 5B is a schematic block diagram indicating full-day relations of the elements from FIG. 5A. Specifically, as discussed above, instances of the intervals of FIG. 5A may be created cyclically in accordance with the alternating operative and nonoperative periods of FIG. 3. In some embodiments, when considering full day data (e.g., data including the nonoperative pre-operative period 310a, nonoperative post-operative period 310d, and all intervening periods), the system may accordingly anticipate a preliminary interval “day start to patient in” 555a to account for actions within the pre-operative period 310a. This interval may, e.g., begin when the first personnel enters the theater for the day and may end when the patient enters the theater for the first surgery. Accordingly, as shown by the arrow 555c, this may result in a transition to the first instance of the “patient in to skin cut” interval 550d. From there, as indicated by the circular relation, the data may be cyclically grouped into instances of the intervals 550a-e, e.g., in accordance with the alternating periods 315a, 310b, 315b, 310c. etc. until the period 315c.

At the conclusion of the final surgery for the day (e.g., surgery 315c), and following the last instance of the interval 550a after that surgery, then rather than continue with additional cyclical data allocations among instances of the intervals 550a-e, the system may instead transition to a final “patient out to day end” interval 555b, as shown by the arrow 555d (which may be used to assess nonoperative post-operative period 310d). The “patient out to day end” interval 555b may end when the last team member leaves the theater or the data acquisition concludes. One will appreciate that various of the disclosed computer systems may be trained to distinguish actions in the interval 555b from the corresponding data of interval 550b (naturally, conclusion of the data stream may also be used in some embodiments to infer the presence of interval 555b). Though concluding the day's actions, analysis of interval 555b may still be appropriate in some embodiments, as actions taken at the end of one day may affect the following day's performance.

### Example Task to Interval Assignments and Action Temporal Intervals

In some embodiments, the durations of each of intervals 550a-e may be determined based upon respective start and end times of various tasks or actions within the theater. Naturally, when the intervals 550a-e are used consecutively, the end time for a preceding interval (e.g., the end of interval 550c) may be the start time of the succeeding interval (e.g., the beginning of interval 550d). When coupled with a task action grouping ontology, theater-wide data may be readily grouped into meaningful divisions for downstream analysis. This may facilitate, e.g., consistency in verifying that team members have been adhering to proposed feedback, as well as computer-based verification of the same, across disparate theaters, team configurations, etc. As will be explained, some task actions may occur over a period of time (e.g., cleaning), while others may occur at a specific moment (e.g., entrance of a team member).

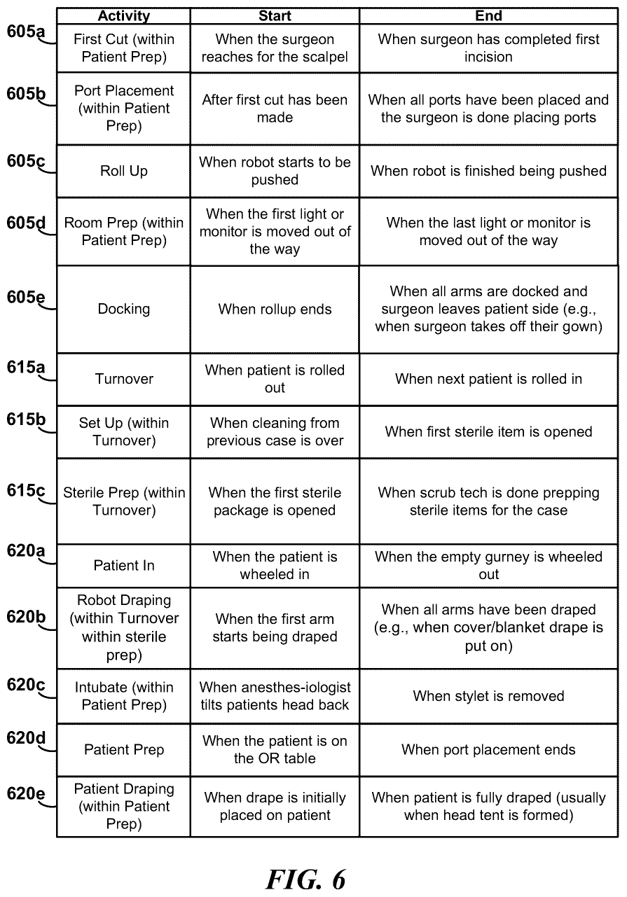

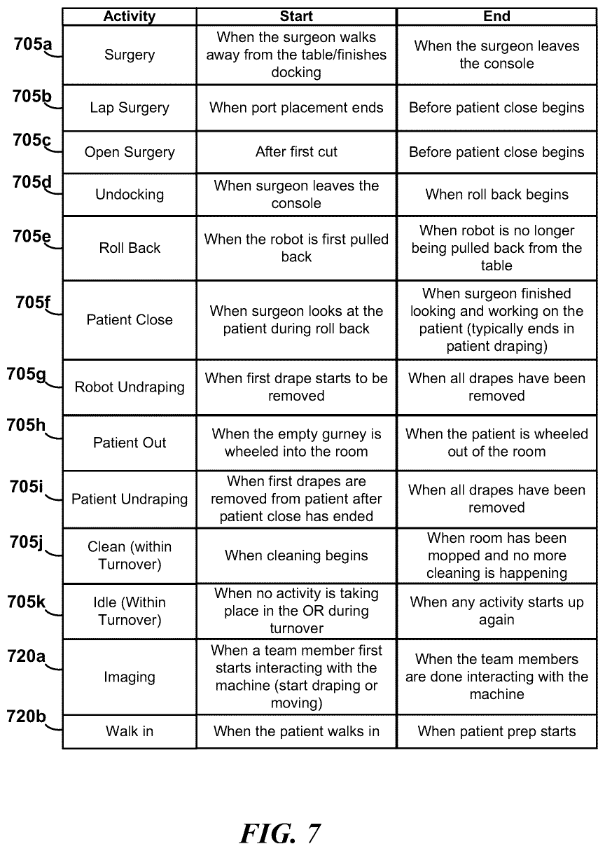

Specifically, FIG. 5C depicts four high-level task action classes or groupings of tasks, referred to for example as phases or stages: post-surgery 520, turnover 525, pre-surgery 510, and surgery 515. Surgery 515 may include the tasks or actions 515a-i. As will be discussed, FIGS. 6 and 7 provide various example temporal definitions for the actions, though for the reader's appreciation, brief summaries will be provided here. Specifically, the task “first cut” 515a, may correspond to a time when the first incision upon the patient occurs (consider, e.g., the duration 605a). The task “port placement” 515b, may correspond to a duration between the time when a first port is placed into the patient and the time when the last port is placed (consider, e.g., the duration 605b). The task “rollup” 515c, may correspond to the duration in which a team member begins moving a robotic system to a time when the robotic system assumes the pose it will use during at least an initial portion of the surgical procedure (consider, e.g., the duration 605c). The task “room prep” 515d, may correspond to a duration beginning with the first surgery preparation action specific to the surgery being performed and may conclude with the last preparation action specific to the surgery being performed (consider, e.g., the duration 605d). The task “docking” 515e, may correspond to a duration starting when a team member begins docking a robotic system and concludes when the robotic system is docked (consider, e.g., the duration 605e). The task “surgery” 515f, may correspond with a duration starting with the first incision and ending with the final closure of the patient (consider, e.g., the durations 705a-c for respective contemplated surgeries, specifically the robotic surgery 705a and non-robotic surgeries 705b and 705c). Naturally, in many taxonomies, these action blocks may be further broken down into considerably more action and task divisions in accordance with the analyst's desired focus (e.g., if the action “port placement” 515b were associated with an inefficiency, a supplemental taxonomy wherein each port's placement were a distinct action, with its own measured duration, may be appropriate for refining the analysis). Here, however, as nonoperative period actions are the subject of review, the general task “surgery” 515f (e.g., one of durations 705a-c) may suffice, despite surgery's encompassing many constituent actions. The task “undocking” 515g, may correspond to a duration beginning when a team member starts to undock a robotic system and concludes when the robotic system is undocked (consider, e.g., the duration 705d). The task “rollback” 515h, may correspond to a duration when a team member begins moving a robotic system away from a patient and concludes when the robotic system assumes a pose it will retain until turnover begins (consider, e.g., the duration 705e). The task “patient close” 515a, may correspond to a duration (e.g., duration 705f) when the surgeon observes the patient during rollback (e.g., one will appreciate by this example that some action durations may overlap and proceed in parallel).

Within the post-surgical class grouping 520, the task “robot undraping” 520a may correspond to a duration when a team member first begins undraping a robotic system and ends when the robotic system is undraped (consider, e.g., the duration 705g). The task “patient out” 520b, may correspond to a time, or duration, during which the patient leaves the theater (consider, e.g., the duration 705h). The task “patient undraping” 520c, may correspond to a duration beginning when a team member begins undraping the patient and ends when the patient is undraped (consider, e.g., the duration 705i).

Within the turnover class grouping 525, the task “clean” 525a, may correspond to a duration starting when the first team member begins cleaning equipment in the theater and concludes when the last team member (which may be the same team member) completes the last cleaning of any equipment (consider, e.g., the duration 705j). The task “idle” 525b, may correspond to a duration that starts when team members are not performing any other task and concludes when they begin performing another task (consider, e.g., the duration 705k). The task “turnover” 505a may correspond to a duration that starts when the first team member begins resetting the theater from the last procedure and concludes when the last team member (which may be the same team member) finishes the reset (consider, e.g., the duration 615a). The task “setup” 505b may correspond to a duration that starts when the first team member begins changing the pose of equipment to be used in a surgery, and concludes when the last team member (which may be the same team member) finishes the last equipment pose adjustment (consider, e.g., the duration 615a). The task “sterile prep” 505c, may correspond to a duration that starts when the first team member begins cleaning the surgical area and concludes when the last team member (which may be the same team member) finishes cleaning the surgical area (consider, e.g., the duration 615c). Again, while shown here in linear sequences, one will appreciate that task actions within the classes may proceed in orders other than that shown or, in some instances, may refer to temporal periods which may overlap and may proceed in parallel (e.g., when performed by different team members).

Within pre-surgery class grouping 510, the task “patient in” 510a may correspond to a duration that starts and ends when the patient first enters the theater (consider, e.g., the duration 620a). The task “robot draping” 510b may correspond to a duration that starts when the a member begins draping the robotic system and concludes when draping is complete (consider, e.g., the duration 620b). The task “intubate” 510c may correspond to a duration that starts when intubation of the patient begins and concludes when intubation is complete (consider, e.g., the duration 620c). The task “patient prep” 510d may correspond to a duration that starts when a team member begins preparing the patient for surgery and concludes when preparations are complete (consider, e.g., the duration 620d). The task “patient draping” 510e may correspond to a duration that starts when a team member begins draping the patient and concludes when the patient is draped (consider, e.g., the duration 620e).

Though not discussed herein, as mentioned, one will appreciate the possibility of additional or different task actions. For example, the durations of “Imaging” 720a and “Walk In” 720b, though not part of the example taxonomy of FIG. 5C, may also be determined in some embodiments.

Thus, as indicated by the respective arrows in FIG. 5C, the intervals of FIG. 5A may be allocated as follows. “Skin-close to patient-out” 550a may begin at the last closing operation 515j of the previous surgery interval and concludes with the patient's departure from the theater (e.g., from the end of the last suture at block 515i until the patient has departed at block 520b). Similarly, the interval “Patient-out to case-open” 550b may begin when the patient's departure from the theater at block 520b and concludes with the start of sterile prep at block 505c for the next case.

The interval “case-open to patient-in” 550c, may begin with the start of the sterile prep at block 505c and conclude with the start of the new patient entering the theater at block 510a. The interval “patient-in to skin cut” 550d may begin when the new patient enters the theater at block 510a and concludes at the start of the first cut at block 515. The surgery itself may occur during the interval 550e as shown.

As previously discussed, the “wheels out to wheels in” interval 550f is the interval from the start of “Patient out to case open” 550b and concludes with the end of “case open to patient in” 550c.

### Example Nonoperative Metric Generation and Scoring

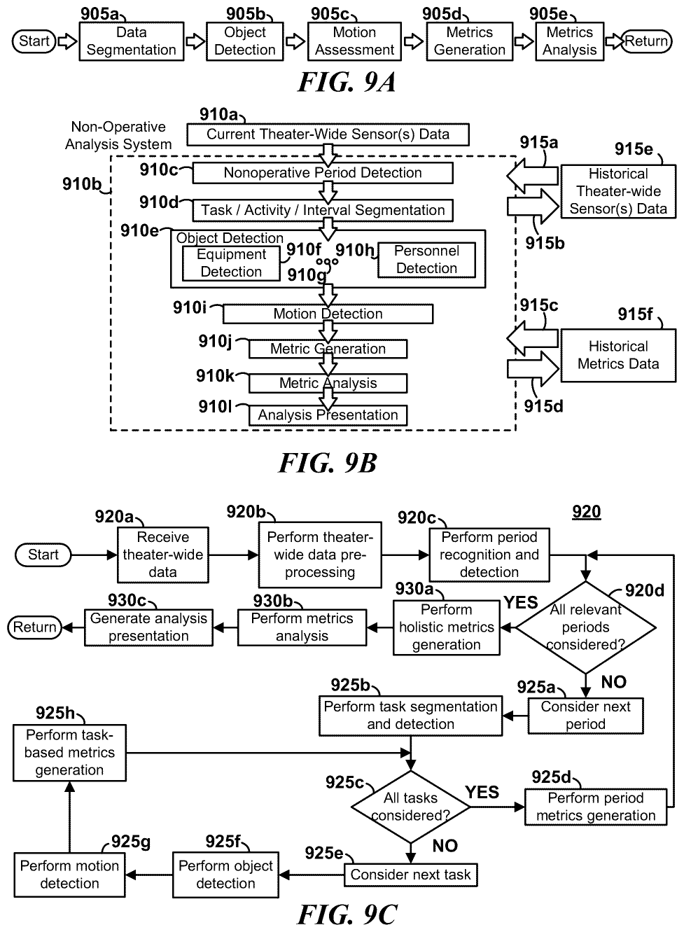

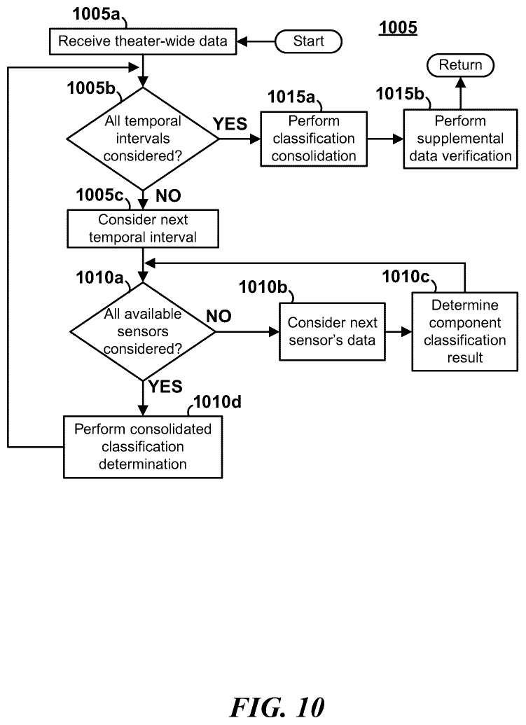

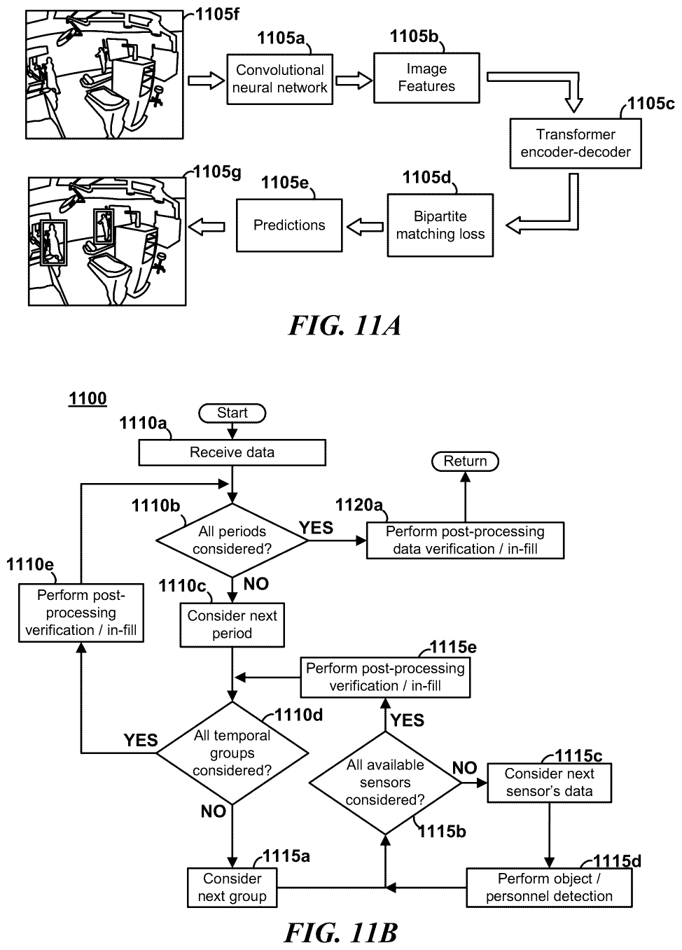

After the nonoperative segments have been identified (e.g., using systems and methods discussed herein with respect to FIGS. 9A-C and FIG. 10), the number and location of objects (e.g., using systems and methods discussed herein with respect to FIGS. 9A-C and FIGS. 11A-B), such as personnel, within each segment, and their respective motions have been identified (e.g., using systems and methods discussed herein with respect to FIGS. 9A-C, 12A-B, 13A-D, and 14), the system may generate one or more metric values. As mentioned, the duration and relative times of the intervals, classes, and task actions of FIGS. 5A-C may themselves serve as metrics.

Various embodiments may also determine “composite” metric scores based upon various of the other determined metrics. These metrics assume the functional form of EQN. 1:

$$s = f ( m ) \qquad ( 1 )$$

where s refers to the composite metric score value, which may be confined to a range, e.g., from 0 to 1, from 0 to 100, etc., and f(·) represents the mapping from individual metrics to the composite score. For example, m may be a vector of metrics computed using various data streams and models as disclosed herein. In such composite scores, in some embodiments, the constituent metrics may fall within one of temporal workflow, scheduling, human resource, or other groupings disclosed herein.

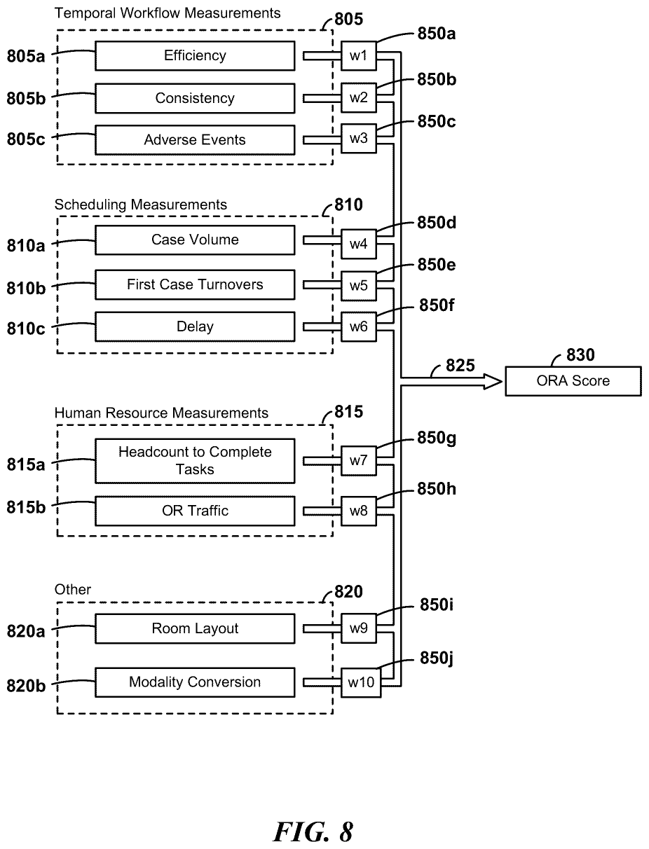

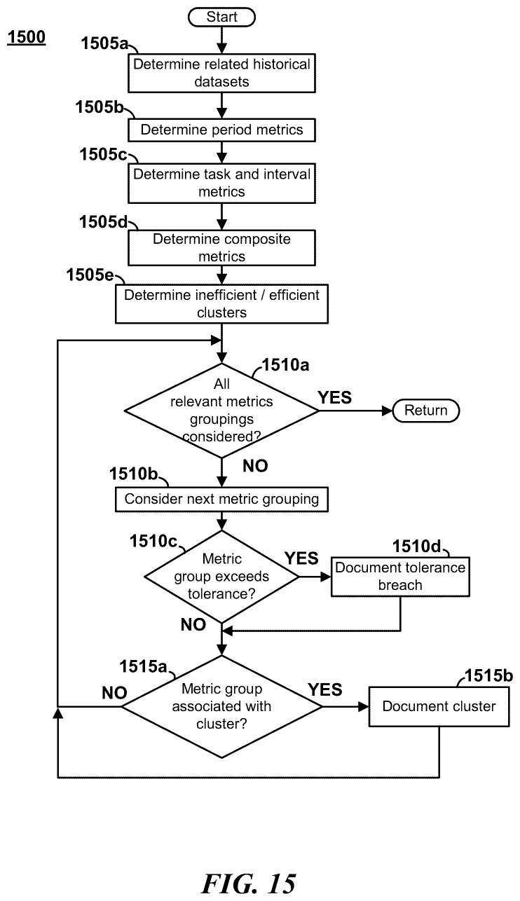

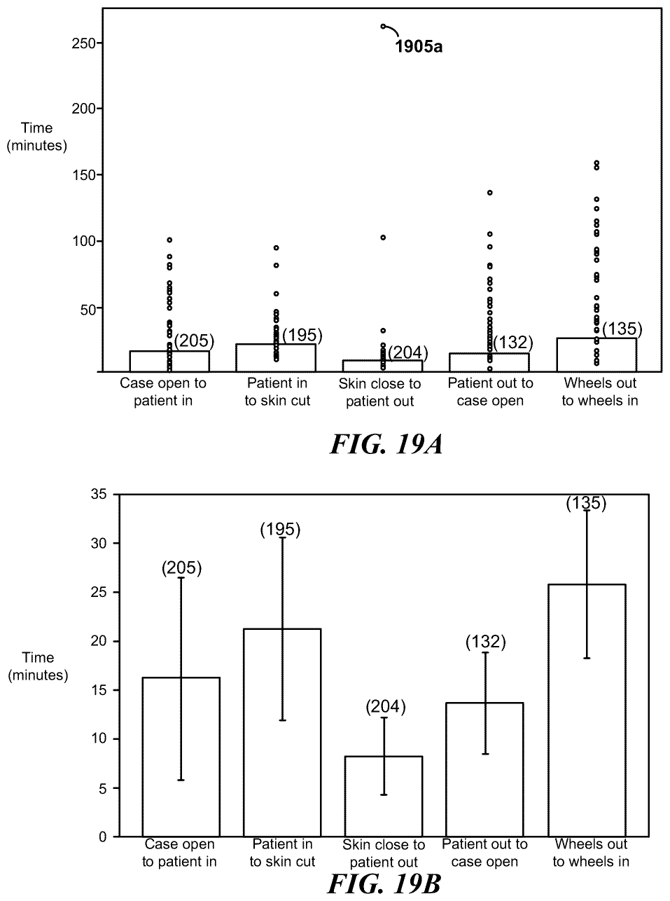

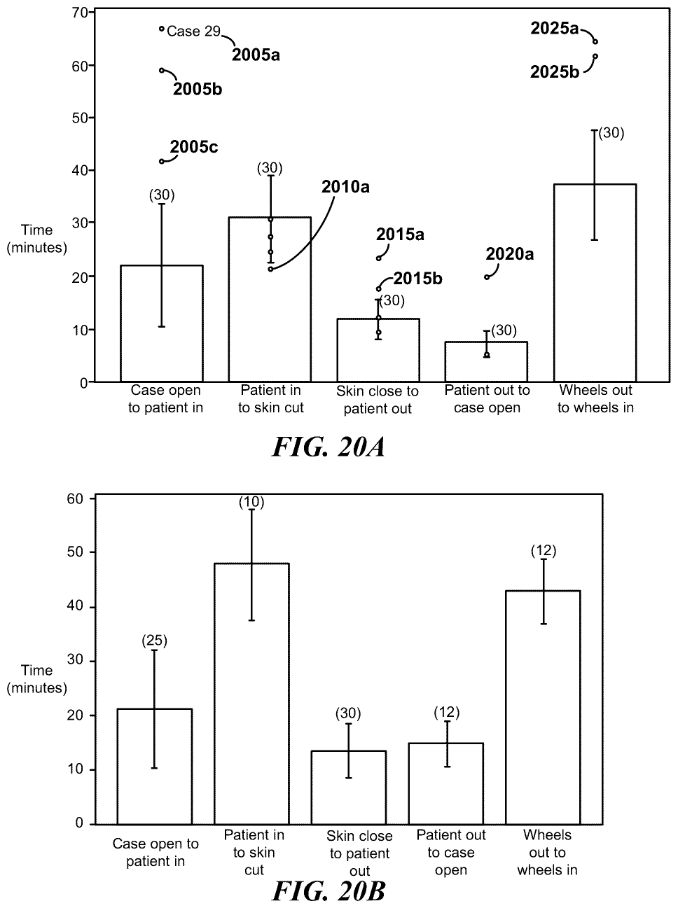

Specifically, FIG. 8 is a schematic block diagram illustrating various metrics and their relations in constructing an “ORA score” as may be performed in some embodiments. Within the temporal grouping 805, an “efficiency” scoring metric 805a may combine the nonoperative metrics that measure temporal workflow efficiency in an OR, e.g., the duration of one or more of the six temporal interval metrics of FIG. 5A. More specifically, the nonoperative metrics, averaged, as a mean or median, over all cases collected from a team, theater, or hospital, may be compared to the top 20% teams, theaters, or hospitals (e.g., as manually indicated by reviewers or from historical patterns via iterations of topology 450) in a database as a benchmark. A “consistency” metric 805b may combine (e.g., sum or find the mean or median) the standard deviations of nonoperative metrics (e.g., the six temporal interval metrics of FIG. 5A) across all cases collected from a current team, theater, or hospital. An “adverse event” metric 805c may combine (e.g., sum) negative outliers, e.g., as detected in terms of the interval metrics of FIGS. 5A-B. Outliers may, e.g., be detected using statistical analysis algorithms (e.g., clustering, distribution analysis, regression, etc. as discussed herein with reference to FIGS. 15, 16, 19A-B, and 20A-B). Negative outliers may be identified as those for which at least one of the nonoperative interval metrics of FIGS. 5A-B metrics are outside a threshold, such as a standard deviation, from than the relevant team, theater, or hospital median or mean (e.g., based on a threshold specified by an expert reviewer or upon historical patterns from past iterations of topology 450). Examples of such outliers are discussed herein, e.g., with respect to FIGS. 19A-B and FIGS. 20A-B.

Within the scheduling grouping 810, a “case volume” scoring metric 810a includes the mean or median number of cases operated per OR, per day, for a team, theater, or hospital, normalized by the expected case volume for a typical OR (e.g., again, as designated in a historical dataset benchmark, such as a mean or median). A “first case turnovers” scoring metric 810b is the ratio of first cases in an operating day that were turned over compared to the total number of first cases captured from a team, theater, or hospital. Alternatively, a more general “case turnovers” metric is the ratio of all cases that were turned-over compared to the total number of cases as performed by a team, in a theater, or in hospital. A “delay” scoring metric 810c is an mean or median positive (behind a scheduled start time of an action) or negative (before a scheduled start time of an action) departure from a scheduled time in minutes for each case, normalized by the acceptable delay (e.g., a historical mean or median benchmark). Naturally, the negative or positive definition may be reversed (e.g., wherein starting late is instead negative and starting early is instead positive) if other contextual parameters are likewise adjusted.

Within the human resource metrics grouping 815, a “headcount to complete tasks” scoring metric 815a combines the mean or median headcount (the largest number of detected personnel throughout the procedure in the OR at one time) over all cases collected for the team, theater, or hospital needed to complete each of the temporal nonoperative tasks for each case, normalized by the recommended headcount for each task (e.g., a historical benchmark median or mean). An “OR Traffic” scoring metric 815b measures the mean amount of motion in the OR during each case, averaged (itself as a median or mean) over all cases collected for the team, theater, or hospital, normalized by the recommended amount of traffic (e.g., based upon a historical benchmark as described above). For example, this metric may receive (two or three-dimensional) optical flow, and convert such raw data to a single numerical value, e.g., an entropy representation, a mean magnitude, a median magnitude, etc.

Within the “other” metrics grouping 815, a “room layout” scoring metric 820a includes a ratio of robotic cases with multi-part roll-ups or roll-backs, normalized by the total number of robotic cases for the team, theater, or hospital. That is, ideally, each roll up or back of the robotic system would include a single motion. When, instead, the team member moves the robotic system back and forth, such a “multi-part” roll implies an inefficiency, and so the number of such multi-part rolls relative to all the roll up and roll back events may provide an indication of the proportion of inefficient attempts. As indicated by this example, some metrics may be unique to robotic theaters, just as some metrics may be unique to nonrobotic theaters. Is some embodiments, correspondences between metrics unique to each theater-type may be specified to facilitate their comparison. A “modality conversion” scoring metric 820b includes a ratio of cases that have both robotic and non-robotic modalities normalized by the total number of cases for the team, theater, or hospital. For example, this metric may count the number of conversions, e.g., transitioning from a planned robotic configuration to a nonrobotic configuration, and vice versa, and then dividing the total number of such cases with such a conversion by the total cases. Whether occurring in an operative or nonoperative periods, such conversions may be reflective of inefficiencies in nonoperative periods (e.g., improper actions in a prior nonoperative period may have rendered the planned robotic procedure in the operative period impractical). Thus, this metric may capture inefficiencies in planning, in equipment, or in unexpected complications in the original surgical plan.

While each of the metrics 805a-c, 810a-c, 815a-c, and 820a-b may be considered individually to assess nonoperative period performances, or in combinations of the multiple of the metrics, as discussed above with respect to EQN. 1, some embodiments consider an “ORA score” 830 reflecting an integrated 825 representation of all these metrics. When, e.g., presented in combination with data of the duration of one or more of the intervals in FIG. 5A-C, the ORA score may provide a readily discernible means for reviewers to quickly and intuitively assess the relative performance of surgical teams, surgical theaters, hospitals and hospital systems, etc. during nonoperative periods, across theaters, across teams, across types of surgical procedures (nonoperative periods before or after prostatectomies, hernia repair, etc.), types of surgical modalities (nonoperative periods preparing for, or resetting after, nonrobotic laparoscopic procedures, nonrobotic open procedures, robotic procedures, etc.), hospital systems, etc.

Accordingly, while some embodiments may employ more complicated relationships (e.g., employing any suitable mathematical functions and operations) between the metrics 805a-c, 810a-c, 815a-c, and 820a-b in forming the ORA score 830, in this example, each of the metrics may be weighted by a corresponding weighting value 850a-j such that the integrating 825 is a weighted sum of each of the metrics. The weights may be selected, e.g., by a hospital administrator or reviewers in accordance with which of the metrics are discerned to be more vital to current needs for efficiency improvement. For example, in a system where reviewers wish to assess whether reports that limited staff are affecting efficiency, then the weight 850g may be upscaled relative to the other weights. Thus, when the ORA score 830 across procedures is compared in connection with the durations of one or more of the intervals in FIG. 5A-C for the groups of surgeries, the reviewer can more readily discern if there exists a relation between the head count and undesirable interval durations. Naturally, one will appreciate other choices and combinations of weight adjustment, as well as particular consideration of specific interval durations, to assess other performance characteristics.

### Example Metric Scoring Methodologies—ORA Significance Assessment

Some higher ORA composite metrics scores may positively correlate with increased system utilization u and reduced OR minutes per case t for the hospitals in a database, e.g., as represented by EQN. 2:

$${p}_{s , u} = \frac{cov ( s , u )}{{σ}_{s} {σ}_{u}} \geq 0.75 \qquad ( 2 )$$

Thus, the ORA composite score may be used for a variety of analysis and feedback applications. For example, the ORA composite score may be used to detect negative trends and prioritize hospitals, theaters, teams, or team members, that need workflow optimizations. The ORA composite score may also be used to monitor workflow optimizations, e.g., to verify adherence to requested adjustments, as well as to verify that the desired improvements are, in fact, occurring. The ORA composite score may also be used to provide an objective measure of efficiency for when teams perform new types of surgeries for the first time.

### Example Metric Scoring Methodologies—Additional Metrics

Additional metrics to assess workflow efficiency may be generated by compositing time, staff count, and motion metrics. For example, a composite score may consider scheduling efficiency (e.g., a composite formed from one or more of case volume 810a, first case turnovers 810b, and case delay 810c) and one or both of modality conversion 820b and the duration of an “idle time” metric, which is a mean or median of the idle time (for individual members or teams collectively) over a period (e.g., during action 525b).

Though, for convenience, sometimes described as considering the behavior of one or more team members, one will appreciate that the metrics described herein may be used to compare the performances of individual members, teams, theaters (across varying teams and modalities), hospitals, hospital systems, etc. Similarly, metrics calculated at the individual, team, or hospital level may be aggregated for assessments of a higher level. For example, to compare hospital systems, metrics for team members within each of the systems, across the system's hospitals, may be determined, and then averaged (e.g., a mean, median, sum weighted by characteristics of the team members, etc.) for a system-to-system comparison.

### Example Nonoperative Data Processing Workflow

FIG. 9A is a schematic block diagram depicting a general processing flow as may be implemented in some embodiments. Specifically, this example flow employs various machine learning consolidation systems for producing elemental OR metrics (such as temporal interval durations, personnel presence, personnel motion, equipment motion, etc., from which other metrics, e.g., as described in FIG. 8, may be generated) from the raw multimodal theater-wide sensor data.

In some embodiments (e.g., where the data has not been pre-processed), a nonoperative segment detection module 905a may be used to detect nonoperative segments from full-day theater-wide data. A personnel count detection module 905b may then be used to detect a number of people involved in each of the detected nonoperative segments/activities of the theater-wide data (e.g., a spatial-temporal machine learning algorithm employing a 3D convolutional network for handing visual image and depth data over time, e.g., as appearing in video). A motion assessment module 905c may then be used to measure the amount of motion (e.g., people, equipment, etc.) observed in each of the nonoperative segment/activities (e.g., using optical flow methods, a machine learning tracking system, etc.). A metrics generation component 905d may then be used to generate metrics, e.g., as disclosed herein (e.g., determining as metrics the temporal durations of each of the intervals and actions of FIGS. 5A-C and the metrics as discussed in FIG. 8). While metrics results may be presented directly to the reviewer in some embodiments, as described herein, some embodiments may instead provide some initial analytical assessment of the metric values, determining standard deviations relative to historical values, prioritizing greater tolerance departures for prioritized presentation to the reviewer, determining if metric values (e.g., motion) indicate that it would be desirable to perform a more refined analysis of the data (e.g., determining team member movement paths, object collision event detections, etc.), etc. Accordingly, a metrics analysis component 905e may then analyze the generated metrics, e.g., to determine outliers relative to historical patterns.

FIG. 9B is a schematic block diagram depicting elements in a more detailed example processing flow than the flow depicted in FIG. 9A, as may be implemented in some embodiments. One will appreciate that each depicted component may be logic or may be one or more machine learning systems, as discussed in greater detail herein. The computer system 910b may receive the theater wide sensor data 910a and first perform the nonoperative period detection 910c (e.g., identifying the periods 310a, 310b, 310c, 310d, though some systems may be configured to only detect nonoperative periods of the character of periods 310b, and 310c). Once the portions of the theater-wide data corresponding to the nonoperative periods have been detected, the data may then be further segmented into corresponding action tasks or intervals (e.g., the intervals 550a-d and/or groupings 510, 515, 520, 525 and respective action tasks) at block 910d.

Using object detection (and in some embodiments, tracking) machine learning systems 910e, the system may perform object detection using machine learning methods, such as of equipment 910f or personnel 910h (ellipsis 910g indicating the possibility of other machine learning systems). In some embodiments, only personnel detection 910h is performed, as only the number of personnel and their motion are needed for the desired metrics. Motion detection component 910i may then analyze the objects detected at block 910e to determine their respective motions, e.g., using various machine learning methods, optical flow, combinations thereof, etc. disclosed herein.

Using the number of objects, detected motion, and determined interval durations, a metric generation system 910j may generate metrics (e.g., the interval durations may themselves serve as metrics, the values of FIG. 8 may be calculated, etc.). The metric values may then be analyzed via component 910k to determine, e.g., outliers and other deviations from historical data (e.g., previous iterations of the topology 450). The system may consider 915a, 915c historical sensor data 915e and historical metrics data 915f when performing the historical comparison at block 910k (e.g., clustering historical metric values around efficient and inefficient nodes, then assessing the newly arrived data's distance to these nodes). In this manner, the system may infer that entire teams, groups of members, or individual members performed subpar compared to historical metrics data for similar roles, team member compositions, or individual team members. Conversely, the processed and raw theater-wide sensor data may be provided 915b to the historical data storage 915e for use in future analysis. Similarly, the metrics results and outlier determinations may be recorded 915d in the historical metrics database 915f for future reference.

The results of the analysis may then be presented via component 910l (e.g., sent over a network to one or more of applications 550f) for presentation to the reviewer. For example, application algorithms may consume the determined metrics and nonoperative data and propose customized actionable coaching for each individual in the team, as well as the team as a whole, based upon metrics analysis results (though such coaching or feedback may first be determined on the computer system 910b in some embodiments). Example recommendations include, e.g.: changes in the OR layout at various points in time, changes in OR scheduling, changes in communication systems between team members, changes in numbers of staff involved in various tasks, etc. In some embodiments, such coaching and feedback may be generated by comparing the metric values to a finite corpus of known inefficient patterns (or conversely, known efficient patterns) and corresponding remediations to be proposed (e.g., slow port placement and excess headcount may be correlated with an inefficiency resolved by reducing head count for that task).

For further clarity, FIG. 9C is a flow diagram illustrating various operations in an example overall process 920 for analyzing theater-wide data. At block 920a, the computer system may receive the theater-wide sensor data for the theater to be examined. At block 920b, the system may perform pre-processing on the data, e.g., reconciling theaterwide data to a common format, as when fisheye and rectilinear sensor data are both to be processed.

At block 920c, the system may perform operative and nonoperative period recognitions, e.g., identifying each of the segments 310a-d and 315a-c from the raw theater wide sensor data. In some embodiments, such divisions may be recognized, or verified, via ancillary data, e.g., console data, instrument kinematics data, etc. (which may, e.g., be active only during operative periods).

The system may then iterate over the detected nonoperative periods (e.g., periods 310a, 310b) at blocks 920d and 925a. In some embodiments, operative periods may also be included in the iteration, e.g., to determine metric values that may inform the analysis of the nonoperative segments, though many embodiments will consider only the nonoperative periods. For each period, the system may identify the relevant tasks and intervals at block 925b, e.g., the intervals, groups, and actions of FIGS. 5A-C.

At blocks 925c and 925e, the system may iterate over the corresponding portions of the theater data for the respectively identified tasks and intervals, performing object detections at block 925f, motion detection at block 925g, and corresponding metrics generation at block 925h. In some embodiments, at block 925f, only a number of personnel in the theater may be determined, without determining their roles or identities. Again, the metrics may thus be generated at the action task level, as well as at the other intervals described in FIGS. 5A-C. In alternative embodiments, the metrics may simply be determined for the nonoperative period (e.g., where the duration of the intervals 550a-e are the only metrics to be determined).

After all the relevant tasks and intervals have been considered for the current period at block 925c, then the system may create any additional metric values (e.g., metrics including the values determined at block 925h across multiple tasks as their component values) at block 925d. Once all the periods have been considered at block 920d the system may perform holistic metrics generation at block 930a (e.g., metrics whose component values depend upon the period metrics of block 925d and block 925h, such as certain composite metrics described herein).

At block 930b, the system may analyze the metrics generated at blocks 930a, 925d, and at block 925h. As discussed, many metrics (possibly at each of blocks 930a, 925h, and 925d) will consider historical values, e.g., to normalize the specific values here, in their generation. Similarly, at block 930b the system may determine outliers as described in greater detail herein, by considering the metrics results in connection with historical values. Finally, at block 930c, the system may publish its analysis for use, e.g., in applications 450f.

### Example Nonoperative Theater-Wide Data Processing—Nonoperative Data Recognition

One will appreciate a number of systems and methods sufficient for performing the operative/nonoperative period detection of components 905a or 910c and activity/task/interval segmentation of block 910d (e.g., identifying the actions, tasks, or intervals of FIGS. 5A-C). Indeed, as mentioned, in some embodiments, alternative signals than the theater-wide data or monitoring of gross-signals in the theater-wide data may suffice for distinguishing periods 310a-d from periods 315a-d. For example, in some embodiments, a team member may provide explicit notification. Similarly, the absence of kinematics and system events data from robotic surgical systemics consoles or instruments may indicate a prolonged separation between the surgeon and patient or between a robotic platform and the patient, which may suffice to indicate that an inter-surgical nonoperative period has begun (or provide verification of a machine learning system's parallel determination).

However, some embodiments consider instead, or in addition, employing machine learning systems for performing the nonoperative period detection. For example, some embodiments employ spatiotemporal model architectures, e.g., like a transformer architecture such as that described in Bertasius, Gedas, Heng Wang, and Lorenzo Torresani. “Is Space-Time Attention All You Need for Video Understanding?” arXiv™ preprint arXiv™:2102.05095 (2021). Such approaches may also be especially useful for automatic activity detection from long sequences of theater-wide sensor data. The spatial segment transformer architecture may be designed to learn features from frames of theater-wide data (e.g., visual image video data, depth frame video data, visual image and depth frame video data, etc.). The temporal segment may be based upon a gated recurrent unit (GRU) method and designed to learn the sequence of actions in a long video and may, e.g., be trained in a fully supervised manner (again, where data labelling may be assisted by the activation of surgical instrument data). For example, OR theater-wide data may be first annotated by a human expert to create ground truth labels and then fed to the model for supervised training.

Some embodiments may employ a two-stage model training strategy: first training the back-bone transformer model to extract features and then training the temporal model to learn a sequence. Input to the model training may be long sequences of theater-wide data (e.g., many hours of visual image video) with output time-stamps for each segment (e.g., the nonoperative segments) or activity (e.g., intervals and tasks of FIGS. 5A-C) of interest. One will appreciate that some models may operate on individual visual images, individual depth frames, groups of image frames (e.g., segments of video), groups of depth frames (e.g., segments of depth frame video), combinations of visual video and depth video, etc.

As another example, FIG. 10 is a flow diagram illustrating various operations in an example process 1005 for performing nonoperative period detection in some embodiments. Specifically, as the number of theater-wide sensors may change across theaters, or across time in the same theater, it may be undesirable to invest in training a machine learning system configured to receive only a specific number of theater-wide data inputs. Thus, in these embodiments, where the classifier is not configured to consider the theater-wide sensor data from all the available streams at once, the system may instead consider the streams individually, or in smaller groups, and then analyze the collective results, e.g., in combination with smoothing operations, so as to assign a categorization to the segment under consideration.

For example, after receiving the theater-wide data at block 1005a (e.g., all of three streams 325a-e, 330a-e, and 335a-e) the system may iterate over the data in intervals at blocks 1005b and 1005c. For example, the system may consider the streams in successive segments (e.g., 30 second, one, or two minute intervals), though the data therein may be down sampled depending upon the framerate of its acquisition. For each interval of data, the system may iterate over the portion of the interval data associated with the respective sensor's streams at blocks 1010a and 1010b (e.g., each of streams 325a-e, 330a-e, and 335a-e or groups thereof, possibly considering the same stream more than once in different groupings). For each stream, the system may determine the classification results at block 1010c as pertaining to an operative or nonoperative interval. After all the streams have been considered, at block 1010d, the system may consider the final classification of the interval. For example, the system may take a majority vote of the individual stream classifications of block 1010c, resolving ties and smoothing the results based upon continuity with previous (and possibly subsequently determined) classifications.

After all the theater-wide data has been considered at block 1005b, then at block 1015a the system may consolidate the classification results (e.g., performing smoothing and continuity harmonization for all the data, analogous to that discussed with respect to block 1010d, but here for larger smoothing windows, e.g., one to two hours). At block 1015b, the system may perform any supplemental data verification before publishing the results. For example, if supplemental data indicates time intervals with known classifications, the classification assignments may be hardcoded for these true positives and the smoothing rerun.

### Example Nonoperative Theater-Wide Data Processing—Object Recognition

Like nonoperative and operative theater-wide data segmentation, one will likewise appreciate a number of ways for performing object detection (e.g., at block 905b or component 910e). Again, in some embodiments, object detection includes merely a number of personnel count, and so a You Only Look Once (YOLO) style network (e.g., as described in Redmon, Joseph, et al. “You Only Look Once: Unified, Realtime Object Detection.” arXiv™ preprint arXiv™:1506.02640 (2015)), perhaps applied iteratively, may suffice. However, some embodiments consider using groups of visual images or depth frames. For example, some embodiments employ a transformer based spatial model to process frames of the theater-wide data, detecting all humans present and reporting the number. An example of such architecture is described in Carion, Nicolas, et al. “End-to-End Object Detection with Transformers.” arXiv™ preprint arXiv™:2005.12872 (2020).

To clarify this specific approach, FIG. 11A is a schematic block diagram illustrating an example information processing flow as may be used for performing object detection in connection with some embodiments. Given a visual or depth frame image 1105f, the system may present the image's raw pixel or depth values to a convolutional network 1105a trained to produce image features 1105b. These features may in turn be provided to a transformer encoder-decoder 1105c and the bipartite matching loss 1105d used to make predictions 1105e for the location and number of objects (e.g., personnel or equipment) in the image, reflected here by bounding boxes within the augmented image 1105g (one will appreciate that an actual augmented image may not be produced by the system, but rather, only indications of the object locations and, in some embodiments, of the type of object found therein).

FIG. 11B is a flow diagram illustrating various operations in an example process 1100 for performing object detection as may be used in connection with some embodiments. At block 1110a, the system may receive the theater-wide data (visual image data, depth data, etc.). At blocks 1110b, and 1110c, as in the process 1005, the system may iterate over the nonoperative periods, considering the data in discrete, successive intervals (as mentioned, in some embodiments the operative periods may be considered as well, e.g., to verify continuity with the object detections and recognitions at the beginnings or ends of the nonoperative periods).

At blocks 1110d and 1115a the system may consider groups of theater-wide data. For example, some embodiments may consider every moment of data capture, whereas other embodiments may consider every other capture or captures at intervals, since some theater sensors may employ high data acquisition rates (indeed, not all sensors in the theater may apply a same rate and so normalization may be applied so as to consolidate the data). For such high rates, it may not be reasonable to interpolate object locations between data captures if the data capture rate is sufficiently larger than the movement speeds of objects in the theater. Similarly, some theater sensor's data captures may not be perfectly synchronized, or may capture data at different rates, obligating the system to interpolate or to select data captures sufficiently corresponding in time so as to perform detection and metrics calculations.

At blocks 1115b and 1115c, the system may consider the data in the separate theater-wide sensor data streams and perform object detection at block 1115d, e.g., as described above with respect to FIG. 11A, or using a YOLO network, etc. After object detection has been performed for each stream for the group under consideration, the system may perform post-processing at block 1115e. For example, if the relative poses of the theater-wide sensors are known within the theater, then their respective object detections may be reconciled to better confirm the location of the object in a three-dimensional representation such as a three-dimensional point cloud. Similarly, the relative data captures may be used to verify one another's determinations and to resolve occlusions based upon temporal continuity (e.g., as when a team member occludes one senor's perspective, but not another sensor's).

After all of the temporal groups have been considered at block 1110d, then at block 1110e, additional verification may be performed, e.g., using temporal information from across the intervals of block 1110d to reconcile occlusions and lacuna in the object detections of block 1115d. Once all the nonoperative periods of interest have been considered at block 1110b, at block 1120a, the system may perform holistic post-processing and verification in-filling. For example, knowledge regarding object presence between periods or based upon a type of theater or operation may inform the expected numbers and relative locations of objects to be recognized. To this end, even though some embodiments may be interested in analyzing nonoperative periods exclusively, the beginning and end of operative periods may help inform or verify the nonoperative period object detections, and may be considered. For example, if four personnel are consistently recognized throughout an operative period, then the system should expect to identify four personnel at the end of the preceding, and the beginning of the succeeding, nonoperative periods.

### Example Nonoperative Theater-Wide Data Processing—Object Tracking

As with segmentation of the raw data into nonoperative periods (e.g., as performed by nonoperative period detection component 910c), and the detection of objects, such as personnel, within those periods (e.g., via component 910e), one will appreciate a number of ways to perform tracking and motion detection. For example, object detection, as described, e.g., in FIG. 11B, in combination with optical flow analysis (with complementary stream perspectives resolving ambiguities) may readily be used to recognize each particular object's movement throughout the theater. As another example, some embodiments may employ multi-object machine learning tracking algorithms, which involve detecting and tracking multiple objects within a sequence of theater-wide data. These approaches may identify and locate objects of interest in each frame and then associate those objects across frames to keep track of their movements over time. For example, some embodiments may use an implementation analogous to that described in Meinhardt, Tim, et al. “TrackFormer: Multi-Object Tracking with Transformers.” arXiv™ preprint arXiv™:2101.02702 (2021).

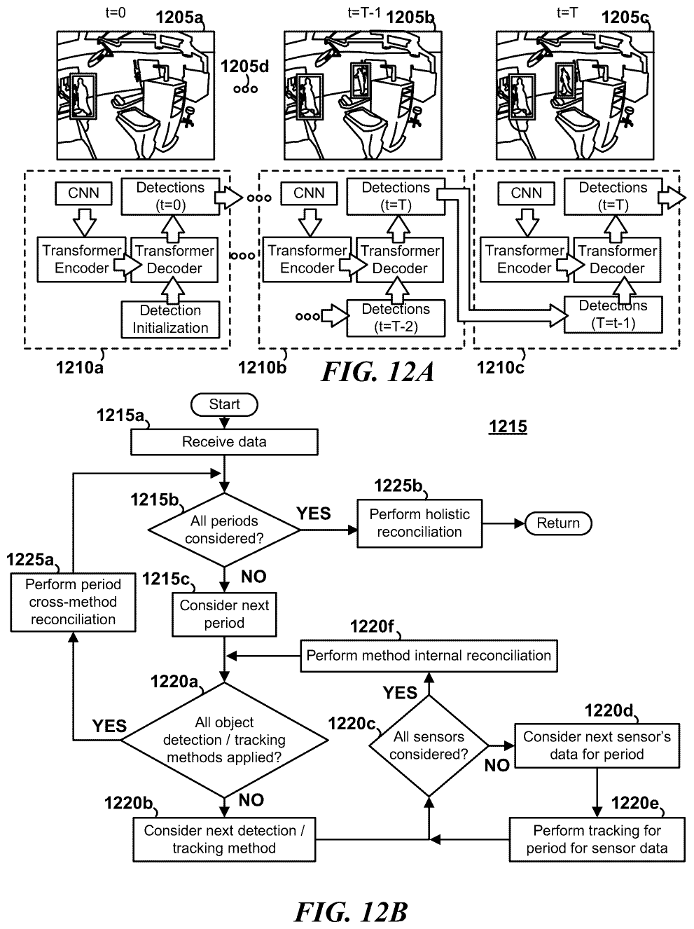

As an example in accordance with the approach of Meinhardt, et al., FIG. 12A is schematic block diagram illustrating an example tracking information processing flow as may be used in connection with some embodiments. In a first visual image or depth frame 1205a, the system may apply a tracking framework collection 1210a of convolution neural network, transformer encoders and decoders, and initial object detection (e.g., with the assistance of the object detection method of FIG. 11A). Iterative application 1210b and 1210c of the tracking framework to subsequent images or frames 1205b and 1205c may produce object detections, such as personnel, with a record of the positions across the frames 1205a, 1205b, 1205c (ellipsis 1205d reflect the presence of intervening frames and tracking recognitions).

FIG. 12B is flow diagram illustrating various operations in an example process 1215 for performing object tracking as may be used in connection with some embodiments. At block 1215a, the system may receive the theater-wide data, e.g., following nonoperative period identification. At blocks 1215b and 1215c the system may iterate over the nonoperative periods and for each period, iterate over the contemplated detection and tracking methods at blocks 1220a and 1220b. For each method, the sensor data streams may be considered in turn at blocks 1220c and 1220d, performing the applicable detection and tracking method at block 1220e (one will appreciate that alternatively, in some embodiments, the streams may be first integrated before applying the object detection and tracking systems, as when simultaneously acquired depth frames from multiple sensors are consolidated into a single virtual model). As mentioned, some methods may benefit from considering temporal and spatial continuity across the theater-wide sensors, and so reconciliation methods for the particular tracking application may be applied at block 1220f.

Similarly, reconciliation between the tracking methods' findings across the period may be performed at block 1225a. For example, determined locations for objects found by the various methods may be averaged. Similarly, the number of objects may be determined by taking a majority vote among the methods, possibly weighted by uncertainty or confidence values associated with the methods. Similarly, after all the nonoperative periods have been considered, the system may perform holistic reconciliation at block 1225b, e.g., ensuring that the initial and final object counts and locations agree with those of neighboring periods or action groups.

As one will note when comparing FIG. 12B and FIG. 9C, object detection, tracking, or motion detection may be performed at the period level (and then associated with tasks/actions/intervals for metrics calculation if desired) or may be performed after the actions, tasks, or intervals have been identified, and upon corresponding data specifically.

### Example Nonoperative Theater-Wide Data Processing—Motion Assessment

While some tracking systems may readily facilitate motion analysis at motion detection component 910i, some embodiments may alternatively, or in parallel, perform motion detection and analysis using visual image and depth frame data. In some embodiments, simply the amount of motion (in magnitude, regardless of its direction component) within the theater in three-dimensional space of any objects, or of only objects of interest, may be useful for determining meaningful metrics during nonoperative periods. However, more refined motion analysis may facilitate more refined inquiries, such as team member path analysis, collision detection, etc.

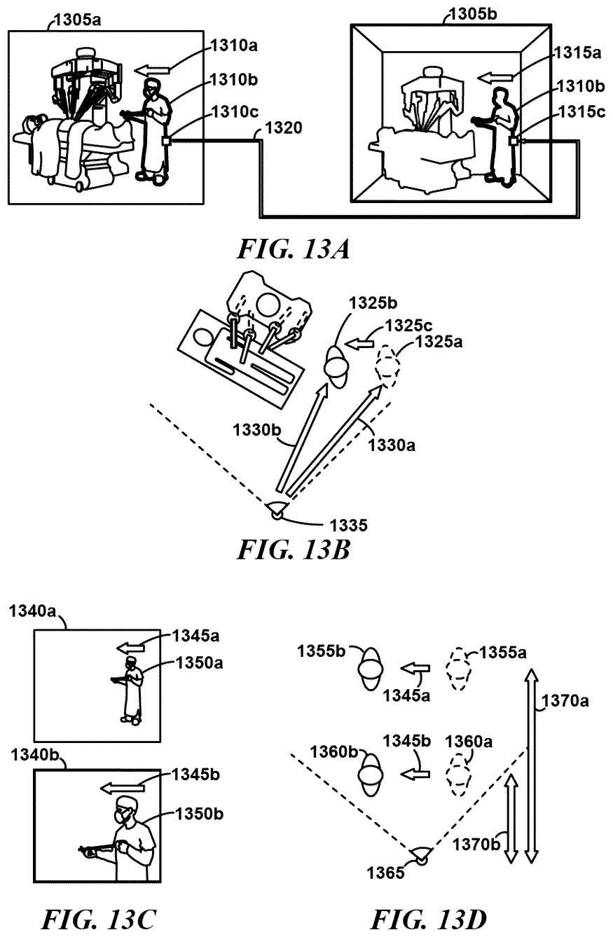

As an example optical-flow based motion assessment, FIG. 13A is a schematic visual image 1305a and depth frame 1305b theater-wide data pair, with an indication of the optical-flow derived correspondence as may be used in some embodiments. Specifically, the data processing system may review sequences of visual image data to detect optical flow. Here, the system has detected that the team member 1310b is moving from the right to the left of the image as indicated by arrow 1310a and by the pixel border around the pixels having optical flow around team member 1310b.

While some embodiments may consider motion based upon the optical flow from visual images alone, it may sometimes be desirable to “standardize” the motion. Specifically, turning to FIG. 13C, movement 1345a far from the camera, as shown in image 1340a may result in a smaller number of pixels (the pixels depicting the member 1350a) being associated with the optical flow. Conversely, as shown in image 1340b, when the team member 1350b is very close to the sensor, their motion 1345b may result in an optical flow affecting many more pixels.

Rather than allow the number of visual image pixels involved in the flow to affect the motion determination, some embodiments may standardize the motion associated with the optical flow to three-dimensional space. That is, with reference to FIG. 13D, the motions 1345a and 1345b may be the same in magnitude in three-dimensional space, as the team members move from locations 1355a, 1360a to locations 1355b, 1360b, respectively. While the locations 1360a-b are a smaller distance 1370b from the sensor 1365 than the distance 1370a from the sensor 1365 to the locations 1355a-b, some embodiments may seek to identify the same amount of motion 1345a, 1345b in each instance. Specifically, downstream metrics may treat the speed of the motions 1345a, 1345b equally, regardless of their distance from the capturing sensor.

To accomplish this, returning to FIG. 13A, for each portion of the visual image 1305a associated with the optical flow, the system may consider the corresponding portions of the simultaneously acquired depth image 1305b, here, where the team member 1310b and their motion, indicated by arrow 1315a, will also be manifest. That is, in this example the pixels 1310c associated with the optical flow may correspond 1320 to the depth values 1315c. By considering these depth values 1320, the system may infer the distance to the object precipitating the optical flow (e.g., one of distances 1370b and 1370a). That is, with reference to FIG. 13B, the system may be able to infer the “standardized” motion 1325c in three-dimensional space for the object moving from position 1325a to position 1325b, once the distances 1330a and 1330b from the capturing sensor 1335 have been inferred from the depth data. In some embodiments, in lieu of first detecting optical flow in the two-dimensional visual image, optical flow in the three-dimensional depth data may instead be used and the standardized motion determined mutatis mutandis.

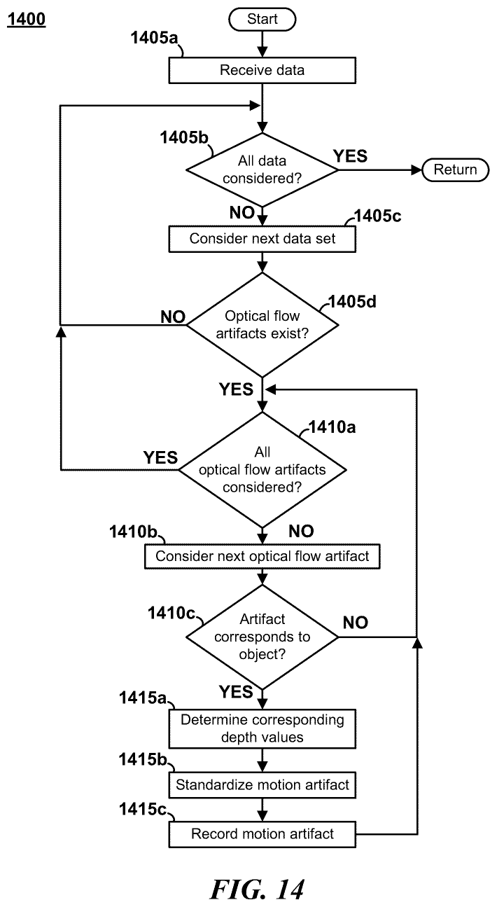

FIG. 14 is a flow diagram illustrating various operations in an example process 1400 for performing motion analysis from theater-wide data, as may be applied in some embodiments. At blocks 1405b and 1405c, the system may iterate over the theater-wide data received at block 1405a. For example, theater-wide data may be down sampled and considered in discrete data sets of temporally successive visual image and depth frame pairs. Where one or more optical flow artifacts (contiguous regions with optical flow above a threshold are detected in either the visual images or the depth frames) are detected within the data set at block 1405d, the system may iterate over the artifacts at blocks 1410a and 1410b. Many artifacts may not correspond to objects of interest for preparing metrics. For example, incidental motion of some equipment, adjustment of some lights, opening of some doors, etc., may not be relevant to the downstream analysis. Accordingly, at block 1410c, the system may verify that the artifact is associated with one or more of the objects of interest (e.g., the personnel or equipment detected using the methods disclosed herein via the machine learning systems of component 910e, e.g., including the systems and methods of FIGS. 11A-B and 12A-B). for example, pixels corresponding to the optical flow may be compared with pixels identified in, e.g., a YOLO network object detection. In some cases, a single optical flow artifact may be associated with more than one object, e.g., when one moving object occludes another moving object. Assessment of the corresponding depth values may reveal the identities of the respective objects appearing in the artifact or at least their respective locations and trajectories.

Thus, where the artifact corresponds to an object of interest (e.g., team personnel), then at block 1415a, the system may determine the corresponding depth values and may standardize the detected motion at block 1415b to be in three-dimensional space (e.g., the same motion value regardless of the distance from the sensor) rather than in the two-dimensional plane of a visual image optical flow, e.g., using the techniques discussed herein with respect to FIGS. 13A-D. The resulting motion may then be recorded at block 1415c for use in subsequent metrics calculation as discussed in greater detail herein.

### Example Nonoperative Theater-Wide Metrics Analysis—Outlier Detection

Following metrics generation (e.g., at metric generation system 910j) some embodiments may seek to recognize outlier behavior (e.g., at metric analysis system 910k) to detect outliers in each team/operating room/hospital/etc. based upon the above metrics, including the durations of the actions and intervals in FIGS. 5A-C, the numbers of people involved in each theater and the amount of motion observed, etc. For example, FIG. 15 is flow diagram illustrating various operations in an example process 1500 for outlier analysis based upon the determined metric values, as may be implemented in some embodiments.

At block 1505a, the system may acquire historical datasets, e.g., for use with metrics having component values (such as normalizations) based upon historical data. At block 1505b, the system may determine metrics results for nonoperative period as a whole (e.g., cumulative motion within the period, regardless of whether it occurred in association with any particular task or interval). At block 1505c, the system may determine metrics results for specific tasks and intervals within each of the nonoperative segments (e.g., the durations of actions and intervals in FIGS. 5A-C). At block 1505d, the system may then determine composite metric values from the previous of the determined metrics (e.g., the ORA score 830 discussed in FIG. 8).

At block 1505e, clusters of metric values corresponding to patterns of inefficient or efficient nonoperative theater states, as well as clusters of metric values corresponding to patterns of efficient or positive nonoperative theater states, may be included in the historical data of block 1505a. Such clusters may be used both to find metric scores, and patterns of metrics scores, distance from ideal clusters and distance from undesirable clusters (e.g., where the distance is the Euclidean distance and each metric of a group is considered as a separate dimension).

Thus, the system may the iterate over the metrics individually, or in groups, at blocks 1510a and 1510b to determine if the metrics or groups exceed a tolerance at block 1510c relative to the historical data clusters (naturally, the nature of the tolerance may change with each expected grouping and may be based upon a historical benchmark, such as one or more standard deviations from a median or mean). Where such tolerance is exceeded (e.g., metric values or groups of metric values are either too close to inefficient clusters or too far from efficient clusters), the system may document the departure at block 1510d for future use in coaching and feedback as described herein.

For clarity, as mentioned, the cluster may occur in an N dimensional space where there are N respective metrics considered in the group (though alternative spaces and surfaces for comparing metric values may also be used). Such an algorithm may be applied to detect outliers for each team/operating room/hospital based upon the above metrics. Cluster algorithms (e.g., based upon K-means, using machine learning classifiers, etc.) may both reveal groupings and identify outliers, the former for recognizing common inefficient/efficient patterns in the values, and the latter for recognizing, e.g., departures from ideal performances or acceptable avoidance of undesirable states.

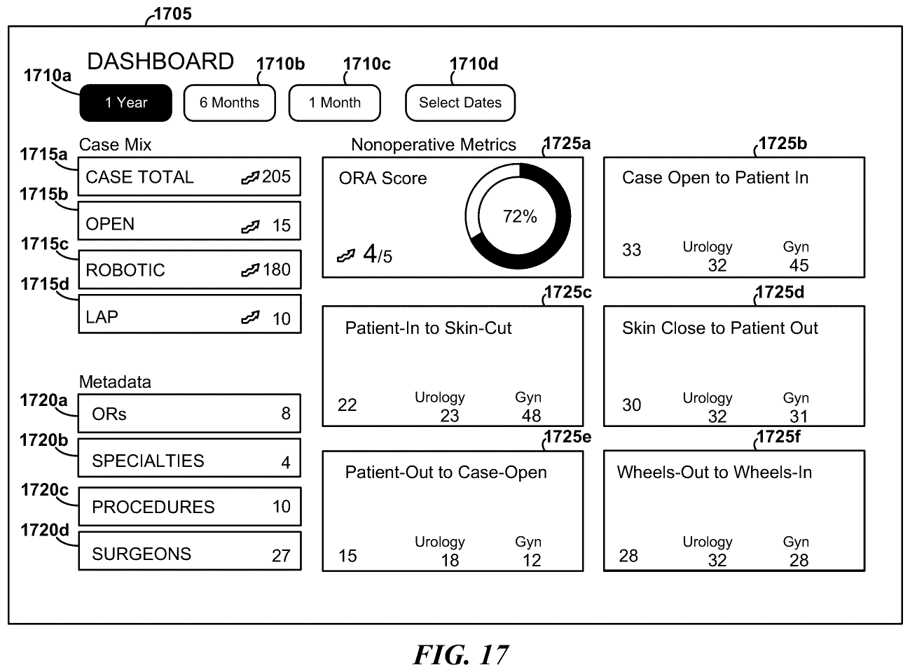

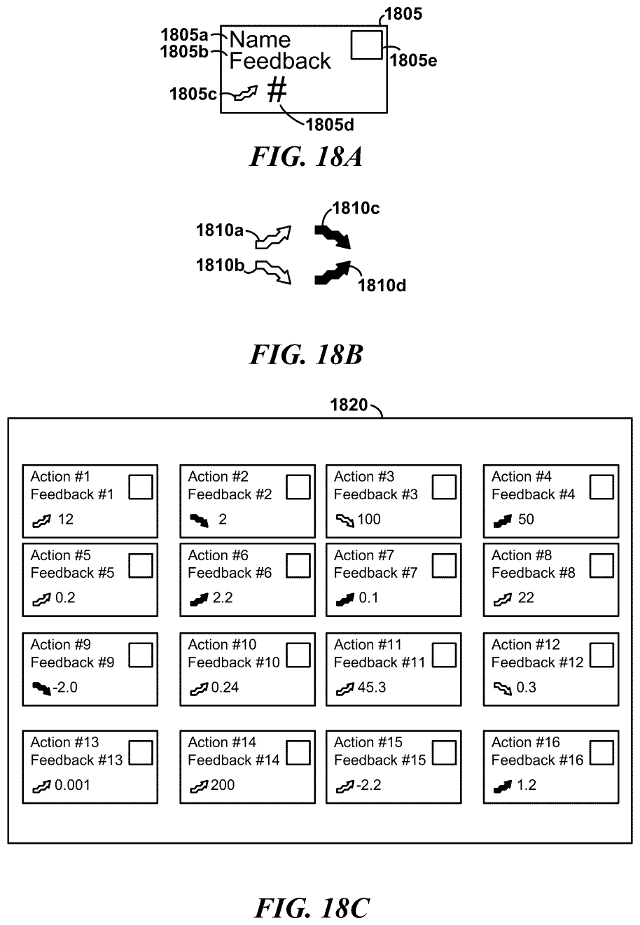

Thus the system may determine whether the metrics individually, or in groups, are associated (e.g., within a threshold distance of, such as the cluster's standard deviation, larges principal component, etc.) with an inefficient, or efficient, cluster at block 1515a, and if so, document the cluster for future coaching and feedback at block 1515b. For example, raw metric values, composite metric values, outliers, distances to or from clusters, correlated remediations, etc., may be presented in a GUI interface, e.g., as will be described herein with respect to FIG. 17 or 18A-C.

### Example Nonoperative Data Analysis—Coaching

Following outlier detection and clustering, in some embodiments, the system may also seek to consolidate the results into a form suitable for use by feedback and coaching (e.g., by the applications 550f). For example, remediating actions may already be known for tolerance breaches (e.g., at block 1510c) or nearness to adverse metrics clusters (e.g., at block 1515a). Here, coaching may, e.g., simply include the known remediation when reporting the breach or clustering association.

Some embodiments may recognize higher level associations in the metric values, from which remediations may be proposed. For example, after considering a new dataset from a theater in a previously unconsidered hospital, various embodiments may determine that a specific surgical specialty (e.g., Urology) in that theater, possesses a large standard deviation in its nonoperative time metrics. Various algorithms disclosed herein may consume such large standard deviations, other data points, and historical data and suggest corrective action regarding with scheduling or staffing model. For example, a regression model may be used that employs historical data to infer potential solutions based upon the data distribution.

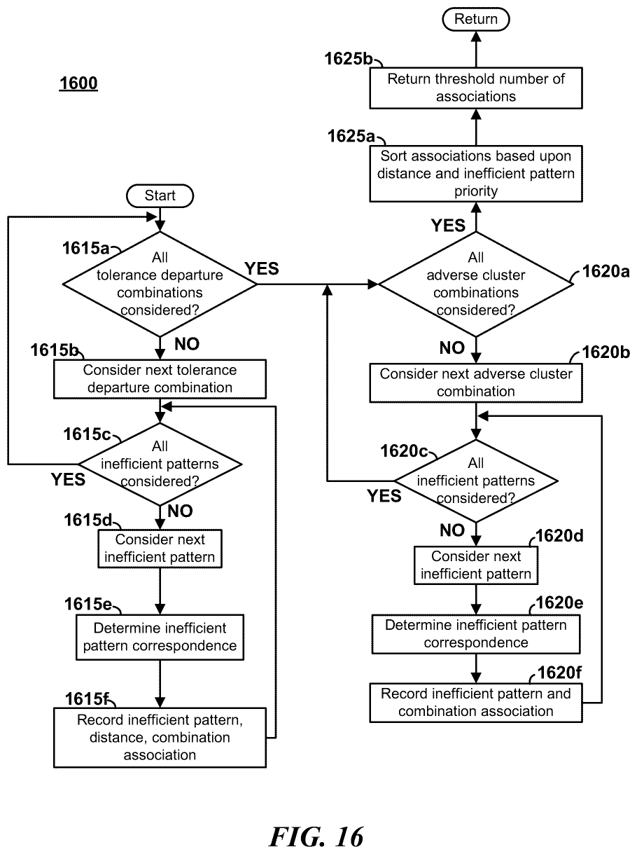

As another example, FIG. 16 is flow diagram illustrating various operations in an example process 1600 for providing coaching feedback based upon the determined metric values, as may be implemented in some embodiments. While focusing on relations between metric values and adverse/inefficient patterns in this example, one will appreciate variations that instead determine relations to desirable/efficient patterns (with corresponding remediations when the metrics depart too far from these preferred states). Similarly, in some embodiments, metrics and groups of metrics may be directly compared to known patterns without first identifying tolerance departures and cluster distances, as in the example process 1600.

Here, at blocks 1615a and 1615b, the system may iterate over all the previously identified tolerance departures (e.g., as determined at block 1510c) for the groupings of one or more metric results and consider whether they correspond with a known inefficient pattern at block 1615c (e.g., taking an inner product with the metric values with a known inefficient vector). For example, a protracted “case open to patient in” duration in combination with certain delay 810c and case volume 810a values, may, e.g., be indicative of a scheduling inefficiency where adjusting the scheduling regularly resolves the undesirable state. Note that the metric or metrics used for mapping to inefficient patterns for remediation may, or may not, be the same as the metric or metrics, which departed from the tolerance (e.g., at block 1615a) or approached the undesirable clustering (e.g., at block 1620a), e.g., the latter may instead indicate that the former may correspond to an inefficient pattern. For example, an outlier in one duration metric from FIG. 5A may imply an inefficient pattern derived from a combinations of metrics from FIG. 8.

Accordingly, the system may iterate through the possible inefficient patterns at blocks 1615c and 1615d to consider how the corresponding metric values resemble the inefficient pattern. For example, the Euclidean distance from the metrics to the pattern may be taken at block 1615e. At block 1615f, the system may record the similarity (e.g., the distance) between the inefficient pattern and the metrics group associated with the tolerance departure.

Similarly, following consideration of the tolerance departures, the system may consider metrics score combinations with clusters near adverse/inefficient events (e.g., as determined at block 1515a) at blocks 1620a and 1620b. As was done previously, the system may iterate over the possible known inefficient patterns at blocks 1620c and 1620d, again determining the inefficient pattern correspondence to the respective metric values (which may or may not be the same group of metric values identified in the cluster association of block 1620a) at block 1620e (again, e.g., the Euclidean or other appropriate similarity metric) and recording the degree of correspondence at block 1620f.

Based upon the distances and correspondences determined at blocks 1615e and 1620e, respectively, the system may determine a priority ordering for the detected inefficient patterns at block 1625a. At block 1625b, the system may return the most significant threshold number of inefficient pattern associations. For example, each inefficient pattern may be associated with a priority (e.g., high priority modes may be those with a potential for causing a downstream cascade of inefficiencies, patient harm, damage to equipment, etc., whereas lower priority modes may simply lead to temporal delays) and presented accordingly to reviewers. Consequently, each association may be scored as a weighted sum of a similarity between the score metric values and metric values associated with inefficient pattern and then weighted by the severity/priority of the inefficient pattern. In this manner, the most significant of the possible failures may be identified and returned first to the reviewer. The iterative nature of topology 450 may facilitate reconsideration and reweighting of the priorities for process 1600 as reviewers observe the impact of the proposed feedback over time. Similarly, the iterations may provide opportunities to identify additional remediation and inefficient pattern correspondences.

### Example GUI Nonoperative Metrics Analysis Feedback Elements

Presentation of the analysis results, e.g., at block 910l, may take a variety of forms in various embodiments. For example, FIG. 17 is a schematic representation of GUI elements in a quick review dashboard interface for nonoperative metrics review as may be implemented in some embodiments. In this example GUI 1705, selectors 1710a-d are provided for the user to select the temporal range of nonoperative period performance data that they wish to analyze. In this example, the user has selected to review the data captured during the past year. Following such a temporal selection, a “Nonoperative Metrics” region, a “Case Mix” region, and a “Metadata” region may be populated with values corresponding to the nonoperative periods for the selected range of data.

The “Case Mix” region may provide a general description of the data filtered from the temporal selection. Here, for example, there are 205 total cases (nonoperative periods) under consideration as indicated by label 1715a. A decomposition of those 205 cases is then provided by type of surgery via labels 1715b-d (specifically, that of the 205 nonoperative periods, 15 were associated with preparation for open surgeries, 180 with preparation for a robotic surgery, and 10 with preparation for a laparoscopic surgery). The nonoperative periods under consideration may be those occurring before and after the 205 surgeries, only those before, or only those after, etc., depending upon the user's selection.

The “Metadata” region may likewise be populated with various parameters describing the selected data, such as the number of ORs involved (8 per label 1720a), the number of specialties (4 per label 1720b), the number of procedure types (10 per label 1720c) and the number of different surgeons involved in the surgeries (27 per label 1720d).

Within the “Nonoperative Metrics” region, a holistic composite score, such as an ORA score, may be presented in region 1725a using the methods described herein (e.g., as described with respect to FIG. 8). Regions 1725b-f may show corresponding statistics for the intervals of FIG. 5A, here, values for various intervals of FIG. 5A.

Some embodiments may also present scoring metrics results comprehensively, e.g., to allow reviewers to quickly scan the feedback and to identify effective and ineffective aspects of the nonoperative theater performance. For example, FIG. 18A is a schematic representation of a GUI element 1805 as may be used for global quick review feedback in some embodiments. Specifically, individual metrics score values, composite metric scores, departures from tolerances, nearness to desirable or undesirable clustering, etc. may be indicated in a numerical region 1805d. The name of the metrics, etc., may be indicated in the name region 1805a and a desired feedback in region 1805b. A quick review icon 1805e may also be included to facilitate ready identification of the nature of the numerical feedback. A quality relation arrow region 1805c may be used to indicate whether the numerical value in region 1805d is above or below an operational point or tolerance, or trending upward or downward over time, and whether this is construed as indicative of improving or decreasing efficiency.

Specifically, FIG. 18B is a schematic representation of arrow elements as may be used in the quality relation arrow region 1805c of FIG. 18A in some embodiments. The arrow may be, e.g., color-coded to indicate whether the value is efficient (e.g., green) or inefficient (e.g., red). Thus, a rising arrow 1810a may indicate that the value in region 1805d is above a lower bound (e.g., when an idle time following successful completion of a task has increased above a historical average). Similarly, the falling arrow 1810b may indicate that the value in region 1805d is below an upper bound (e.g., when an preparation time has decreased below a historical average). Conversely, a falling arrow 1810c may indicate that the value in region 1805d is below a desired minimum value (e.g., when a number of personnel ready for a required step is below a historical average). Similarly, the rising arrow 1810d may indicate that the value in region 1805d is above a desired upper bound (e.g., when an preparation time has increased beyond a historical average).

By associating relational value both with the arrow direction and highlighting (such as by color, bolding, animation, etc.), reviewers may readily scan a large number of values and discern results indicating efficient or inefficient feedback. Highlighting may also take on a variety of degrees (e.g., alpha values, degree of bolding, frequency of an animation, etc.) to indicate a priority associated with an efficient or inefficient value. For example, FIG. 18C is a schematic representation of GUI elements in a quick review feedback interface 1820 as may be used in some embodiments. Here, the individual quick review feedbacks (instances of the element 1805) may be arranged in a grid and sized so that the reviewer may perceive multiple items at one time. Each element may be selectable, presenting details for the value determination, including, e.g., the corresponding historical data, theater-wide data, intermediate metrics calculation results, etc. One will appreciate that the figure is merely schematic and each “Action” or “Feedback” text may be replaced with one of the metrics described herein (e.g., a duration of intervals from FIGS. 5A-C) and remediations, respectively (though in some configurations the feedback may be omitted from all or some of the elements).

FIG. 19A is a plot of example analytic values as acquired in connection with a prototype implementation of an embodiment. Specifically, FIG. 19A shows results following processing for various of the intervals of FIG. 5A. Here, an outlier value 1905a clearly indicates a deviation in the “skin close to patient out” interval from the median duration of −10 minutes (taking instead approximately −260 minutes). FIG. 19B is similarly a plot of example operating room analytic values as acquired in connection with a prototype implementation of an embodiment. Here, standard deviation intervals may be shown to guide the reviewer in recognizing outlier values (e.g., whether they reflect a longer or shorter duration than the standard deviation interval).

FIG. 20A is a plot of example values as acquired in connection with a prototype implementation of an embodiment. Even without performing the outlier detection and inefficient pattern recognition methods disclosed herein, one can readily determine by inspection that various of the values are outliers. For example, within the “case open to patient in” interval, the cases 2005a-c clearly indicate outliers above the standard deviation. For the “patient in to skin cut” interval, the case 2010a is shorter than the standard deviation interval. For the “skin close to patient out” interval, the cases 2015a-b were outside the standard deviation for the selected historical cases. For the “patient out to case open” interval, the case 2020a lies far outside the standard deviation for the selected historical cases. For the “wheels out to wheels in” interval, the cases 2025a and 2025b lie outside the standard deviation for the selected historical cases. FIG. 20B is similarly a plot of example operating room analytic values as acquired in connection with a prototype implementation of an embodiment.

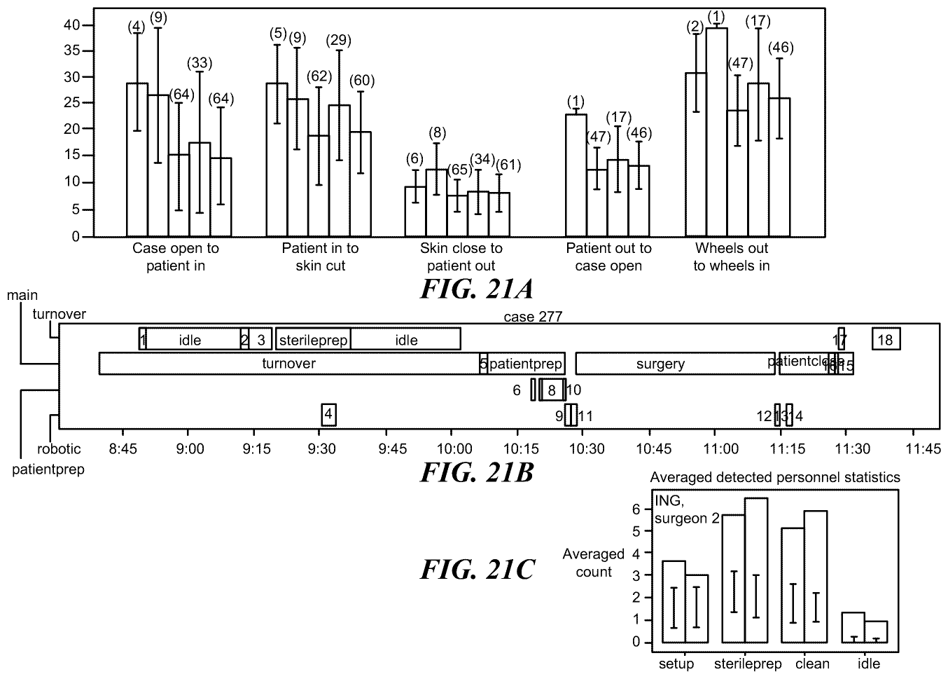

Similarly, FIG. 21A is a plot of example operating room analytic values as acquired in connection with an example prototype implementation of an embodiment. FIG. 21B is a plot of example operating room analytic values as acquired in connection with an example prototype implementation of an embodiment, in a horizontal format. FIG. 21C is also a plot of example operating room analytic values as acquired in connection with an example prototype implementation of an embodiment.

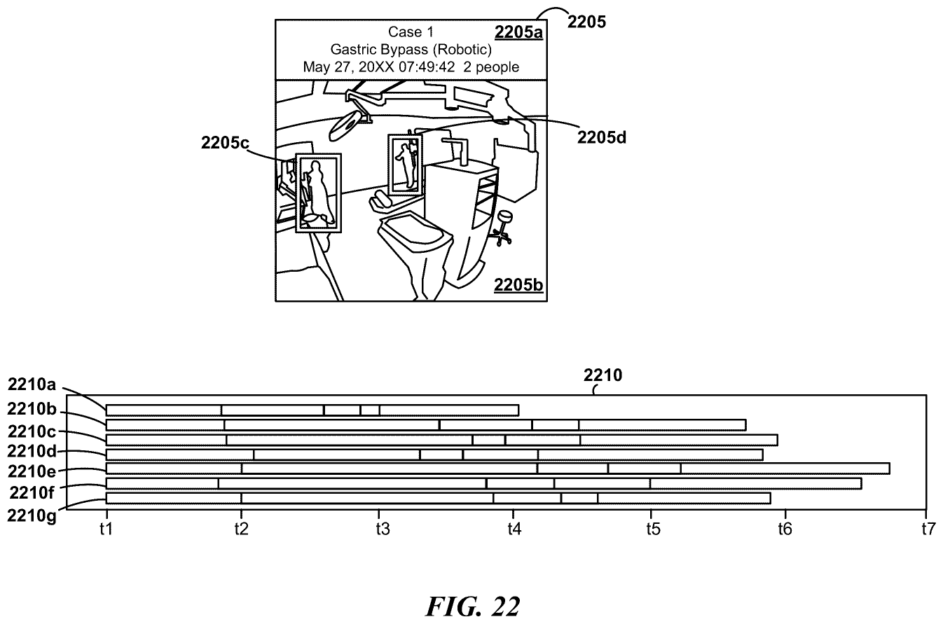

FIG. 22 is a schematic representation of example elements in a graphical user interface for providing metrics-derived feedback, as may be used in some embodiments. In this example, the interface includes two elements: a theater-wide sensor playback element 2205; and a consolidated timeline element 2210 depicting the durations of various intervals within a plurality of nonoperative periods. For example, each of temporal interval breakdowns 2210a-g may indicate the durations of intervals 550a-d for ready comparison (though seven periods are shown in this example, one may readily envision variations with many more rows, as well as more instances of playback element 2205).

Within the theater-wide sensor playback element 2205 may be a metadata section 2205a indicating the identity of the case (“Case 1”), the state of the theater (though a surgical operation “Gastric Bypass”, is shown here, in anticipation of the upcoming surgery, the nonoperative actions and intervals of FIG. 5A-C may be shown here additionally or alternatively), the date and time of the data acquisition (“May 27, 20XX 07:49:42”) and the number of identified personnel (here “2” as determined, e.g., in accordance with component 910h and, e.g., the methods of FIGS. 11A-B). The theater-wide sensor playback element 2205 may also display the data from one or more theater-wide sensors in the playback section 2205b with bounding boxes (e.g., boxes 2205c and 2205d), overlays, outlines, or other suitable indications of the personnel detected. In the consolidated timeline element 2210 a plurality of temporal intervals 2210a-g may be rendered, indicating, e.g., a plurality of interval durations (e.g., the durations of intervals 550a-d for seven nonoperative periods). The playback in the region 2205 may correspond to a selection of one of the intervals in the temporal interval breakdowns 2210a-g (e.g., depicting corresponding theater-wide data playback for that interval). In this manner, the reviewer may readily perceive a corpus of results while simultaneously analyzing the state of a specific instance (e.g., as may have been called to the user's attention based upon, e.g., correspondingly determined metric values or pattern similarities).

Screenshots and Materials Associated with Prototype Implementations of Various Embodiments

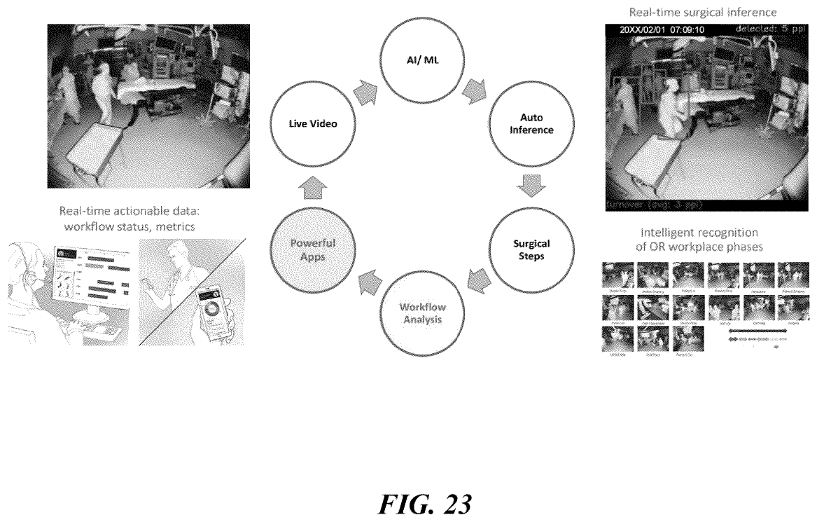

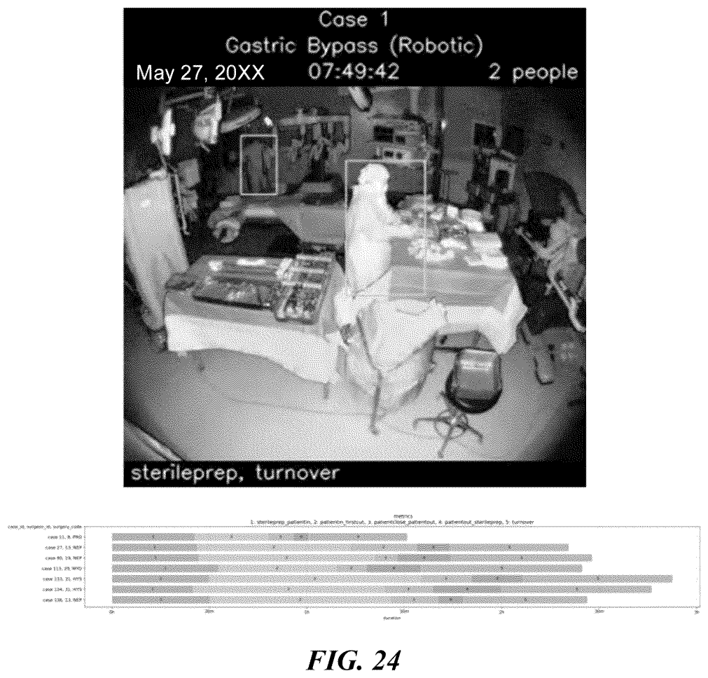

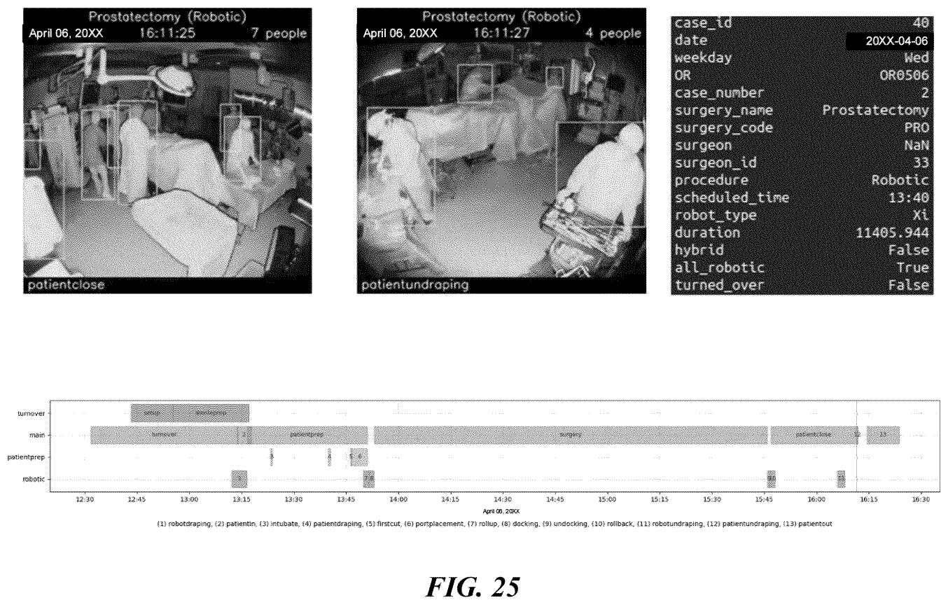

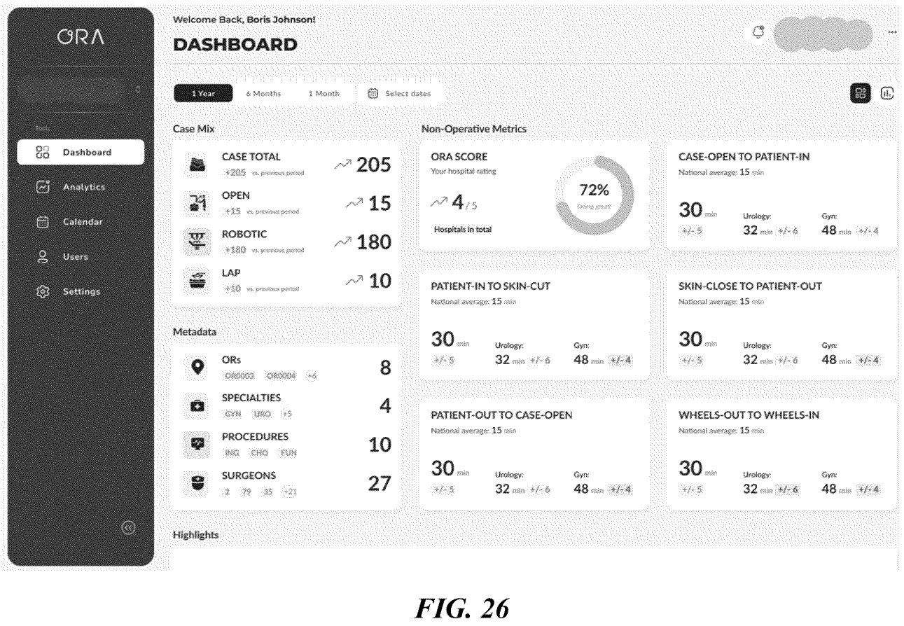

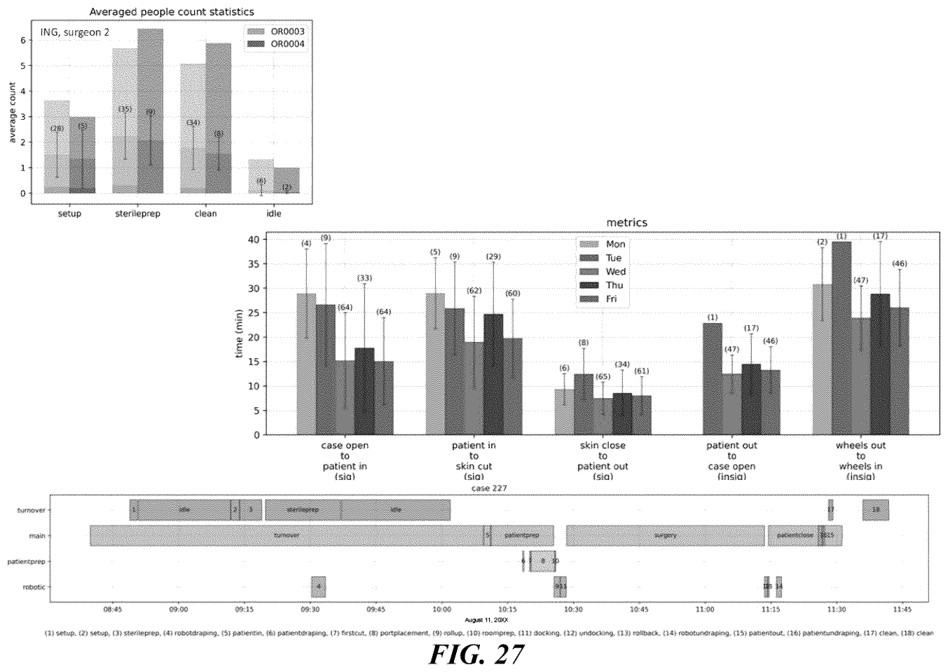

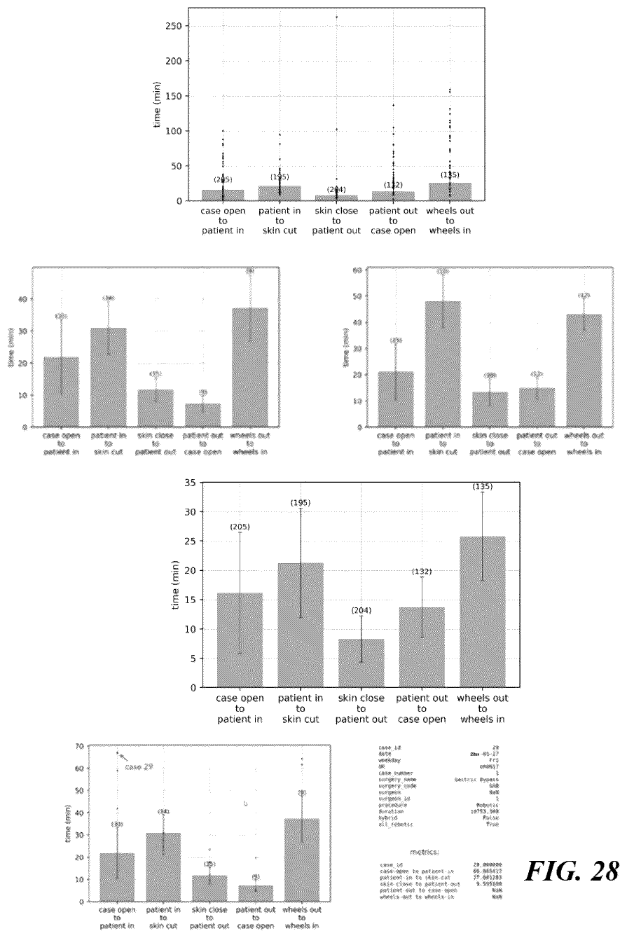

FIG. 23 is an example schematic data processing overview diagram corresponding to aspects of FIG. 4, as may be used in connection with some embodiments. FIG. 24 is a screenshot of a feedback interface corresponding to aspects of FIG. 22, as may be used in connection with some embodiments. FIG. 25 is a screenshot of a feedback interface corresponding to aspects of FIG. 22, as may be used in connection with some embodiments. FIG. 26 is a screenshot of a feedback interface corresponding to aspects of FIG. 17, as may be used in connection with some embodiments. FIG. 27 is a collection of color image plots for example metric values corresponding to aspects of FIGS. 21A-C, as acquired in connection with an example prototype implementation of an embodiment. FIG. 28 is a collection of color plots corresponding to aspects of the plots of FIGS. 19A-B and 20A-B. One will appreciate that dates appearing in the screenshots of FIGS. 23-25, 27, 28 refer to the date of data capture. Accordingly, to better ensure privacy, each instance is here replaced with 20XX.

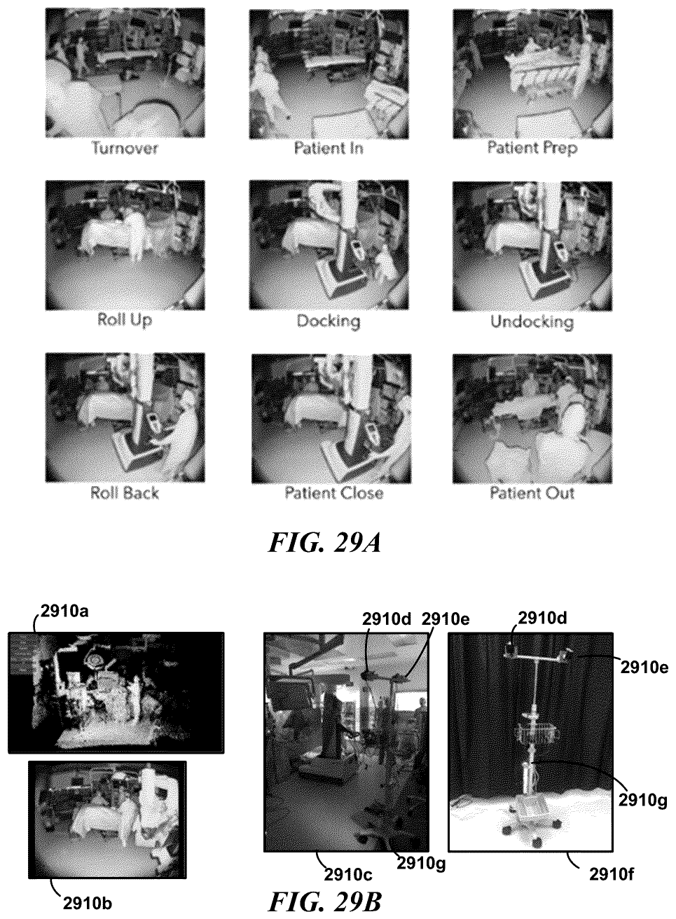

FIG. 29A is a collection of photographs of theater-wide sensor depth and image frames captured in a surgical theater during various of the tasks. FIG. 29B is a collection of theater-wide sensor images captured of a surgical theater during deployment of an example prototype implementation of an embodiment and related photographs of an example theater-wide sensor platform. Specifically, image 2910a depicts a depth frame acquired from a theater-wide sensor wherein the depth values have been color coded to facilitate the reader's visualization. A visual image 2910b acquired from another theater-wide sensor is also provided. In photograph 2910a an elevated stand 2910g for mounting two theater-wide sensors 2910d and 2910e is shown. The image 2910f shows the elevated stand 2910g and sensors 2910d, 2910e from a second perspective.

### Computer System

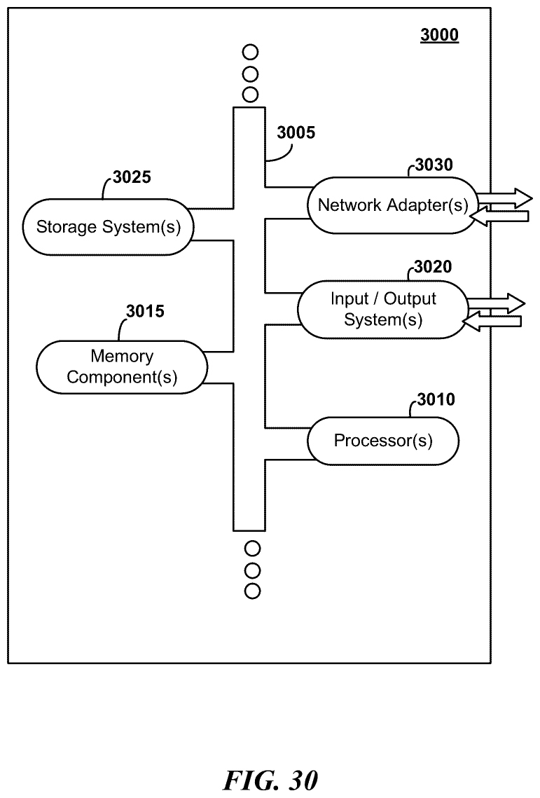

FIG. 30 is a block diagram of an example computer system 3000 as may be used in conjunction with some of the embodiments. In some examples, each of the processing systems 450b can be implemented using the computing system 3000. In some examples, the application 450f can be executed using the computing system 3000. The computing system 3000 may include an interconnect 3005, connecting several components, such as, e.g., one or more processors 3010, one or more memory components 3015, one or more input/output systems 3020, one or more storage systems 3025, one or more network adaptors 3030, etc. The interconnect 3005 may be, e.g., one or more bridges, traces, busses (e.g., an ISA, SCSI, PCI, I2C, Firewire bus, etc.), wires, adapters, or controllers.

The one or more processors 3010 may include, e.g., a general-purpose processor (e.g., x86 processor, RISC processor, et.c), a math coprocessor, a graphics processor, etc. The one or more memory components 3015 may include, e.g., a volatile memory (RAM, SRAM, DRAM, etc.), a non-volatile memory (EPROM, ROM, Flash memory, etc.), or similar devices. The one or more input/output devices 3020 may include, e.g., display devices, keyboards, pointing devices, touchscreen devices, etc. The one or more storage devices 3025 may include, e.g., cloud-based storages, removable Universal Serial Bus (USB) storage, disk drives, etc. In some systems memory components 3015 and storage devices 3025 may be the same components. Network adapters 3030 may include, e.g., wired network interfaces, wireless interfaces, Bluetooth™ adapters, line-of-sight interfaces, etc.

One will recognize that only some of the components, alternative components, or additional components than those depicted in FIG. 30 may be present in some embodiments. Similarly, the components may be combined or serve dual-purposes in some systems. The components may be implemented using special-purpose hardwired circuitry such as, for example, one or more ASICs, PLDs, FPGAs, etc. Thus, some embodiments may be implemented in, for example, programmable circuitry (e.g., one or more microprocessors) programmed with software and/or firmware, or entirely in special-purpose hardwired (non-programmable) circuitry, or in a combination of such forms.

In some embodiments, data structures and message structures may be stored or transmitted via a data transmission medium, e.g., a signal on a communications link, via the network adapters 3030. Transmission may occur across a variety of mediums, e.g., the Internet, a local area network, a wide area network, or a point-to-point dial-up connection, etc. Thus, “computer readable media” can include computer-readable storage media (e.g., “non-transitory” computer-readable media) and computer-readable transmission media.

The one or more memory components 3015 and one or more storage devices 3025 may be computer-readable storage media. In some embodiments, the one or more memory components 3015 or one or more storage devices 3025 may store instructions, which may perform or cause to be performed various of the operations discussed herein. In some embodiments, the instructions stored in memory 3015 can be implemented as software and/or firmware. These instructions may be used to perform operations on the one or more processors 3010 to carry out processes described herein. In some embodiments, such instructions may be provided to the one or more processors 3010 by downloading the instructions from another system, e.g., via network adapter 3030.

For clarity, one will appreciate that while a computer system may be a single machine, residing at a single location, having one or more of the components of FIG. 30, this need not be the case. For example, distributed network computer systems may include multiple individual processing workstations, each workstation having some, or all, of the components depicted in FIG. 30. Processing and various operations described herein may accordingly be spread across the one or more workstations of such a computer system. For example, one will appreciate that a process amenable to being run in a single thread upon a single workstation may instead be separated into an arbitrary number of sub-threads across one or more workstations, such sub-threads then run in serial or in parallel to achieve a same, or substantially similar, result as the process run within the single thread. Similarly, one will appreciate that while a non-transitory computer readable medium may stand alone (e.g., in a single USB storage device), or reside within a single workstation (e.g., in the workstation's random access memory or disk storage), such a medium need not reside at a single geographic location, but may include, e.g., multiple memory storage units residing across geographically separated workstations of a computer system in network communication with one another or across geographically separated storage devices.

### Hierarchical Display Structure for GUIs

With the advent of high-power computing and large-scale data collection and storage capabilities, access to an abundance of data is continuously improving. However, optimal representation of such data to facilitate discovery of efficiencies and inefficiencies in hospital or OR processes is an entirely different task. Optimal representation and summarization of information of a plurality of medical procedures, such as data collected and derived from hospital or OR processes or operations in the manner described herein, can maximize insights gained from the information of a plurality of medical procedures and expedite discovery of such insights in real-time or at a later time. Due to the complex nature of the procedures, the high stakes of the operations, the number of medical staff involved, etc., efficiency and inefficiency information for information of a plurality of medical procedures within the context of hospitals and ORs may be difficult to reveal if presented in a sub-optimal manner. For example, optimizing the size of a care team and room layout affect the amount of foot traffic within the OR and is in turn linked to the reduced risk of infections. In addition, reducing turnover time and improving access and utilization of the robotic system by optimizing scheduling process, leads to reduced anesthesia time as well as cost for hospitals and patients. In other words, the presentation and summarization of information of a plurality of medical procedures is as important as the information itself.

The arrangements disclosed herein can generate information of a plurality of medical procedures that can be provided to a surgeon in real time and to a consultant, student, and staff subsequently for education and further analysis. While the theater-wide data and the information of a plurality of medical procedures (e.g., nonoperative metrics) determined using the machine learning algorithms can provide a tremendous amount of insight to users, which have not been previously available, navigating this wealth of information to obtain the most relevant information efficiently, sometimes in real-time, can pose significant challenges without an effective user interface and methods for selecting/aggregating information for presentation. As discussed in further details herein, information can be presented according to user identity (e.g., roles of the users) such that the information presented to a user is most relevant to that user and can be easily understood by that user. That is a first user with a first role (e.g., surgeon) and a second user with a second role (e.g., consultant) can be presented information at different levels of the hierarchical structure, where the information present at each level can be obtained or derived from the theater-wide sensor data. Furthermore, the hierarchical structure as described herein can optimize space efficiency of an interface that is displayed on a screen on a computer, personal device (e.g., a smart phone), or a screen located within a medical environment. Given that such devices may have limited screen size due to other utilities or the designated deployment within a medical environment, screen size can be an important aspect to optimize.

In some embodiments, the information of a plurality of medical procedures including the analysis results (e.g., at blocks 9101, 930c, and so on) can be presented using a GUI with a hierarchical display structure. For example, the GUI elements, graphical elements, metric values, dashboard layouts, interfaces, graphs, plots, and screenshots shown and described with respect to FIGS. 17-22 and 24-29B are displayed using the hierarchical display structure, as described in further details herein. Such hierarchical display structure optimizes the GUI by improving the representation and summarization of surgery-related data, in various settings such as hospitals, consultants, classroom, and ORs.

As described herein a system (e.g., the computing systems 190a and 190b, the processing systems 450b, or the system 3000) can receive and digest data sources or data streams including one or more of case metadata, three-dimensional point cloud data, RGB image/video data, surgical robot data, and so on. For example, the data sources or data streams can be acquired by the system during real-time acquisition at 450a, received at 910a, 915e, 920a, 1005a, 1110a, 1215a, 1405a, and so on.

In some examples, the case metadata can be displayed in the metadata section 2205a of the theater-wide sensor playback element 2205. In some examples, the case metadata can be displayed in the “Metadata” region of the GUI 1705. In some embodiments, case metadata includes at least one of identifying information of the plurality of medical procedures, identifying information of one or more ORs in which the plurality of medical procedures are performed, identifying information of one or more hospitals in which the plurality of medical procedures are performed, identifying information of medical staff by which the plurality of medical procedures are performed, identifying information of one or more robotic systems or instruments used in the plurality of medical procedures, statistical information of the one or more ORs, statistical information of the one or more hospitals, statistical information of the medical staff, or statistical information of the one or more robotic systems or instruments.

In some examples, the identifying information of the plurality of medical procedures includes at least one of a name or type of each of the plurality of medical procedures, a time at which or a time duration in which each of the plurality of medical procedures is performed, or a modality of each of the plurality of medical procedures. In some examples, the identifying information of the one or more ORs includes a name of each of the one or more ORs. In some examples, the identifying information of the one or more hospitals includes a name of each of the one or more hospitals. In some examples, the identifying information of the medical staff includes a name of each of one or more surgeons. In some examples, the identifying information of the one or more robotic systems or instruments includes at least one of a name of each of the one or more robotic systems or instruments or an attribute of each of the one or more robotic systems or instruments. In some examples, the identifying information of at least one sensor includes at least one of a name of each of the at least one sensor or a modality of each of the at least one sensor.

In some examples, the statistical information of the plurality of medical procedures includes a number of the plurality of medical procedures or a number of types of the plurality of medical procedures performed in the one or more hospital, in the one or more ORs, by the medical staff, or using the one or more robotic systems or instruments. In some examples, the statistical information of the one or more ORs includes a number of the plurality of medical procedures or a number of types of the plurality of medical staff performed in each of the one or more ORs. In some examples, the statistical information of the one or more hospitals includes a number of the plurality of medical procedures or a number of types of the plurality of medical staff performed in each of the one or more hospitals. In some examples, the statistical information of the medical staff includes a number of the plurality of medical procedures or a number of types of the plurality of medical staff performed by the medical staff. In some examples, the statistical information of the one or more robotic systems or instruments includes a number of the plurality of medical procedures or a number of types of the plurality of medical staff performed by the one or more robotic systems or instruments.

In some examples, examples of the medical staff include surgeons, nurses, support staff, and so on, such as the patient-side surgeon 105a and the assisting members 105b. Examples of the robotic systems include the robotic medical system or the robot surgical system described herein. Examples of instruments include the mechanical instrument 110a or the visualization tool 110b. Examples of the modality of a medical procedure (or a modality of a surgical theater) include robotic (e.g., using at least one robotic system), non-robotic laparoscopic, non-robotic open, and so on.

In some examples, case metadata can be stored in a memory device (e.g., the memory component 3015) or a database. The memory device or the database can be provided for a scheduling or work allocation application that schedules hospital or OR processes and operations. For example, a user can input using an input system (e.g., of the input/output system 3020) the case metadata, or the case metadata can be automatically generated using an automated scheduling application. The case metadata can be associated with other types of the information of a plurality of medical procedures such as the three-dimensional point cloud data, RGB image/video data, robot data, and so on. For example, other types of the information of a plurality of medical procedures captured for the same procedure time or scheduled time, in the same OR, with the same procedure name, with the same robot or instrument, or so on can be associated with the corresponding case metadata and can be processed together and displayed using the hierarchical structure together, in the same or different interfaces of the GUI.

The three-dimensional point cloud data is determined using theater-wide data (e.g., depth data, depth frame, or depth frame data) collected using theater-wide sensors (e.g., depth-acquiring sensors). In some examples, the three-dimensional point cloud data can be generated by inputting the theater-wide data into at least one of suitable extrapolation methods, mapping methods, and machine learning models. For example, the depth data for a depth-acquiring sensor with a certain pose can indicate distance measured between the depth-acquiring sensor and points on objects and/or intensity value of the points on objects. Depth data from multiple depth-acquiring sensors with different poses as shown and described relative to FIGS. 2B, 2C, and 3 can be fused into a higher accuracy dataset through registration of depth-acquiring sensors. An intensity value can indicate a reflected signal strength for a point in the three-dimensional point cloud or an object in the three-dimensional point cloud. The three-dimensional point cloud data can define a three-dimensional point cloud or a three-dimensional point cloud representation corresponding to the OR or the theater that can be used to track the location and the number of objects such as the medical staff (personnel) and equipment, for example, using the method described in FIG. 11A. The three-dimensional point cloud includes pixels, each of which is defined by an intensity value (e.g., a gray-scale intensity value) and a three-dimensional coordinates of that pixel with respect to a coordinate frame of the depth-acquiring sensor.

In some examples, the theater-wide sensors, such as the sensors 170a and 170c, can include at least one visual image sensor or a multi-modal sensor that can collect and output images and/or videos, such as color (RGB) image or video data and/or grayscale image or video data. Examples of robot data include data for a robotic system, such as kinematics data, system events data of a robotic system, input received by the console of the robotic system from a user, and timestamps associated therewith.

The system (e.g., the systems 190a, 190b, 450b, and 3000) can execute computer vision algorithms that process the three-dimensional point cloud data and provide one or more of temporal activities data and human actions data associated with procedures in the OR, sometimes performed using a robotic system. In some examples, the system can perform temporal activity recognition to recognize temporal activities data, including phases and activities within a nonoperative or inter-operative period. Examples of a nonoperative period include the nonoperative periods 310a, 310b, 310c, 310d. In some embodiments, the nonoperative periods can be detected at 910c and 920c. Examples of a task within a nonoperative period include the tasks 320a, 320b, 320c, 320d, 320f, and 320e. As described herein, two or more tasks can be grouped as a phase or a stage. Examples of a phase include post-surgery 520, turnover 525, pre-surgery 510, and surgery 515, and so on. Accordingly, the data streams obtained from the theater-wide sensors can be segments into a plurality of periods, including operative periods and nonoperative periods. Each nonoperative periods can include at least one phase. Each phase includes at least one task.

In some examples, to obtain the human actions data, the system can perform human detection to detect at least one individual (e.g., personnel, a medical staff member, a patient, and so on) in each frame of the data collected by the theater-wide sensor. For example, at 910h, personnel detection can be performed by the machine learning systems 910e or at 925f to determine a number of personnel and their motion to determine one or more metrics as described herein. In some examples, the motion detection component 910i can then analyze the objects (including the equipment at 910f and the personnel at 910h) detected at block 910e to determine their respective motions, e.g., using various machine learning methods, optical flow, combinations thereof, etc. disclosed herein.

The system (e.g., the systems 190a, 190b, 450b, and 3000) processes the case metadata, the temporal activities data, and the human actions data to determine metrics (e.g., nonoperative metrics) and statistics. The statistics include a number of personnel involved in completion of each task or phase of the non-operative period, which is computed from the number of personnel detected in each frame of the output of the theater-wide sensor. The case metadata, the temporal activities data, the human actions data, the metrics, and the statistics can be collectively referred to as procedure-related information. The system can determine the metrics based on the activities of personnel, equipment, patient, and so on as evidence in the temporal activities data and the human actions data. The system displays the information of a plurality of medical procedures using an output device (e.g., a display).

Examples of the output device or display include on one or more of the display 125, 150, and 160a, a display that outputs information for the applications 450f, and display communicably coupled to the processing systems 190a, 190b, and 450b, display device or touch screen device of the input/output devices 3020, and so on. In other words, the information of a plurality of medical procedures presented using the hierarchical format can be displayed using displays 125, 150, 160a, etc. that can be located within the surgical theaters 100a and 100b for realtime feedback to the medical staff during hospital or OR processes. The information of a plurality of medical procedures presented using the hierarchical format can be displayed using displays for the applications 450f that can be located remote from the surgical theaters 100a and 100b to provide discovery of information to consultants and students study and analyzing the information of a plurality of medical procedures at any time after or concurrent with the hospital or OR processes and to remote support staff providing realtime assistance to the hospital or OR processes. The information of a plurality of medical procedures presented using the hierarchical format can be displayed using displays of the backend processing systems 190a, 190b, and 450b to provide realtime or ad hoc monitoring and analysis of the information of a plurality of medical procedures by technical or medical staff remote from the surgical theaters 100a and 100b.

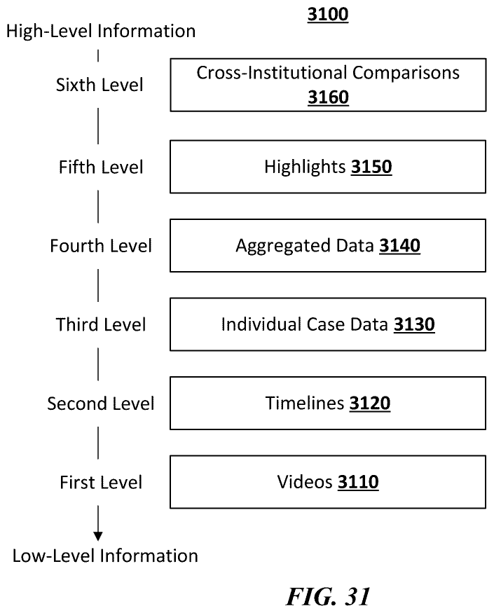

FIG. 31 is an example schematic diagram illustrating an example hierarchical structure 3100 for displaying information of a plurality of medical procedures, according to some embodiments. The information of a plurality of medical procedures can be displayed using a hierarchical design, e.g., the hierarchical structure 3100, to facilitate discovery of efficiencies (corresponding to positive metric values) and inefficiencies (corresponding to negative metric values) in hospital or OR processes for workflow optimization, room layout optimization, care team size optimization, management of scheduling, and so on. For example, the information of a plurality of medical procedures is presented on the output device or display in an interactive, layered format where the highest level of information (e.g., general information) is presented first, and lower-level information (e.g., detailed or specific information) is presented in response to user input that interacts with or selects user interactive elements (e.g., selectors, prompts, and so on) corresponding to aspects of the higher-level information displayed on an interface.

As shown in FIG. 31, the hierarchical structure 3100 includes six levels: highlights 3150, aggregates statistics 3140, individual case data 3130, timelines 3120, and videos 3110, in that order from the highest level of the hierarchical structure 3100 to the lowest level of the hierarchical structure 3100.

In some embodiments, an interface or GUI containing lower level information (referred to as a lower level interface, a lower level view, or lower level GUI) is displayed in response to a user selecting a user interactive element (e.g., a selector, a prompt, and so on) on an interface or GUI containing higher level information (referred to as a higher level interface, a higher level view, or higher level GUI). In some examples, higher level information displayed on a higher level GUI can be configured as a user interactive element. In some examples, a user interactive element can be displayed adjacent to, on, overlapping, or linked to higher level information displayed on a higher level GUI. By selecting such user interactive element, the user can trigger the display of the lower level GUI that contains more detailed information on such higher level information. In some embodiments, a lower level GUI is displayed in response to determining a trigger event while a higher level GUI is displayed. Examples of the trigger event includes a predetermined period of time has passed since the higher level GUI has been first displayed. Thus, a higher level GUI is displayed before a lower level GUI is displayed.

In some examples, a higher level GUI presents higher level information (e.g., metrics, statistics, and so on) calculated, aggregated, generated, or otherwise determined from or based at least in part on lower level information presented on a lower level GUI. Lower level information displayed on a lower level GUI is determined using fewer steps of calculation or aggregation (based on the theater-wide data collected by the theater-wide sensors) than those used to determine higher level information displayed on a higher level GUI. Thus, a lower level GUI contains more detailed or complete information than a higher level GUI.

In some embodiments, a sixth level of a user interface is configured to display the cross-institutional comparisons 3160. The cross-institutional comparisons 3160 are selected portions of the aggregated data 3140 for two or more institutions (e.g., hospitals, hospital groups, and so on) that can be compared or juxtaposed side-by-side, for example, in a single interface (e.g., a screen). That is, a subset of the multiple types of aggregated data 3140 is displayed in the sixth level of the user interface.

In some embodiments, a fifth level of a user interface is configured to display the highlights 3150. The highlights 3150 are selected portions of the aggregated data 3140. That is, a subset of the multiple types of aggregated data 3140 is displayed in the fifth level of the user interface.

In some embodiments, a fourth level of a user interface is configured to display the aggregated data 3140. The aggregated data 3140 includes at least one of aggregated metrics or aggregated statistics. The aggregated data 3140 is determined based on or aggregated (combined) from the case data 3130 of at least one of two or more medical procedures, two or more phases, two or more tasks, two or more ORs, two or more hospitals, two or more robotic systems or instruments, or two or more medical staff members. For example, the aggregated metrics can be computed or aggregated from individual metrics by adding two or more individual metrics, averaging (to determine the mean, median, and standard deviations for) two or more individual metrics, or running two or more individual metrics into a function or algorithm to determine the aggregated metrics. In some examples, the aggregated metrics include composite metric scores (e.g., ORA score, composite OR metric, and so on), such as that determine using EQN. 1. For example, the aggregated statistics can be computed or aggregated from individual statistics by adding two or more individual statistics, averaging (to determine the mean, median, and standard deviations for) two or more individual statistics, or running two or more individual statistics into a function or algorithm to determine the aggregated statistics.

For example, the aggregated data 3140 for a phase can be determined using the individual case data 3130 for multiple tasks of that phase. The aggregated data 3140 for a medical procedure can be determined using the individual case data 3130 for multiple phases of that medical procedure. The aggregated data 3140 for an OR can be determined using the individual case data 3130 for multiple medical procedures performed in that OR. The aggregated data 3140 for a hospital can be determined using the individual case data 3130 for multiple ORs in that hospital. The aggregated data 3140 for a hospital group can be determined using the individual case data 3130 for multiple hospitals in that hospital group.

In some embodiments, the individual case data 3130 for a phase, task, medical procedure, OR, hospital, robotic system or instrument, medical staff member is displayed in response to a user selecting a user interactive element corresponding to the aggregated data 3140 determined based at least in part of the individual case data 3130 for that phase, task, medical procedure, OR, hospital, robotic system or instrument, medical staff member. In some examples, in the fourth level of the user interface, the aggregated data 3140 can be configured as a user interactive element, or a user interactive element can be displayed adjacent to, on, overlapping, or linked to the aggregated data 3140. In some embodiments, individual case data 3130 for a phase, task, medical procedure, OR, hospital, robotic system or instrument, medical staff member can be displayed in the third level of the user interface in response to a user selecting a user interactive element (e.g., a selector, a prompt, and so on) for the aggregated data 3140 displayed on the fourth level of the user interface. For example, selecting the aggregate data 3140 can trigger the display of constituent elements, referred to as the individual case data 3130, that are used to determine the aggregate data 3140.

In some embodiments, a third level of a user interface is configured to display the individual case data 3130. The case data 3130 includes statistics determined based on the case metadata for at least one of at least one medical procedure, at least one phase, at least one task, at least one OR, at least one hospital, at least one robotic system or instrument, or at least one medical staff member. In other words, the metrics and the statistics are determined for different types of the case metadata, such as for each phase, task, medical procedure, OR, hospital, robotic system or instrument, medical staff member, and so on.

In some embodiments, timeline 3120 for a phase, task, medical procedure, OR, hospital, robotic system or instrument, medical staff member is displayed in response to a user selecting a user interactive element corresponding to the case data 3130 for that phase, task, medical procedure, OR, hospital, robotic system or instrument, medical staff member. In some examples, in the third level of the user interface, the case data 3130 can be configured as a user interactive element, or a user interactive element can be displayed adjacent to, on, overlapping, or linked to the case data 3130. In some embodiments, a timeline 3120 for a phase, task, medical procedure, OR, hospital, robotic system or instrument, medical staff member can be displayed in the second level of the user interface in response to a user selecting a user interactive element (e.g., a selector, a prompt, and so on) for the case data 3130 of that phase, task, medical procedure, OR, hospital, robotic system or instrument, medical staff member displayed on the third level of the user interface.

In some examples, the metrics or metric values of the case data 3130 can be determined in the manner described herein. Examples of the metrics include the metrics 805a, 805b, 805c, 810a, 810b, 810c, 815a, 815b, 820a, 820b. In some examples, the individual case data 3130 includes individual metric scores or constituent metric scores. The statistics of the case data 3130 for a phase, task, or medical procedure include a number of personnel or medical staff members involved in completion of each phase of a nonoperative period, each task in a phase, or a medical procedure that includes one or more nonoperative periods. The statistics can be computed from the number of medical staff members detected in each frame of the output of the theater-wide sensor in the manner described herein.

In some embodiments, a second level of a user interface is configured to display the timelines 3120. The timelines 3120 can include timelines for one or more of at least one medical procedures, for at least one period (e.g., nonoperative periods, operative periods, and so on), for at least one phase, for at least one task, for at least one OR, for at least one hospital, for at least one robotic system or instrument, for at least one medical staff member, and so on. That is, the timelines 3120 can illustrate a duration (corresponding to the length of a part of the timeline) of time intervals corresponding to one or more of the at least one medical procedures, for at least one period (e.g., nonoperative periods, operative periods, and so on), for at least one phase, for at least one task, for at least one OR, for at least one hospital, for at least one robotic system or instrument, for at least one medical staff member, and so on. Examples of the timelines 3120 include the consolidated timeline element 2210.

In some embodiments, a video 3110 for a period, a phase, or a task can be displayed in the first level of the user interface in response to a user selecting a user interactive element (e.g., a selector, a prompt, and so on) for that period, phase, or task in a timeline 3120 displayed on a second level of the user interface. In some examples, each period, phase, and task in the timeline 3120 can be configured as a user interactive element, or a user interactive element can be displayed adjacent to, on, overlapping, or linked to each period, phase, and task in the timeline 3120. Each period, phase, and task is shown to have a length that indicates a duration of a time interval for the period, phase, and task. The video 3110 for each period, phase, and task has the same duration as indicated. In some embodiments, a video 3110 for an OR, hospital, robotic system or instrument, or staff member can be displayed in the first level of the user interface in response to a user selecting a user interactive element (e.g., a selector, a prompt, and so on) for that OR, hospital, robotic system or instrument, or staff member displayed on a second level of the user interface.

In some embodiments, a first level of a user interface is configured to display the videos 3110. In some examples, the videos 3110 can include the three-dimensional point cloud representation (e.g., the three-dimensional point cloud data) that represents activities occurring in tasks, phases, and nonoperative periods of medical procedures. In some examples, the video 3110 can include a two-dimensional video rendered from the three-dimensional point cloud data based on suitable mapping algorithms that maps the three-dimensional point cloud data to a series of two-dimensional frames.

In some examples, the videos 3110 include feather-wide data from the theater-wide sensors (e.g., the sensors 170a and 170c) such as visual image video data, depth frame video data, visual image and depth frame video data, etc. The videos 3110 can be two-dimensional frames that can be used to determine the three-dimensional point cloud representation (e.g., the three-dimensional point cloud data) that represents activities occurring in tasks, phases, and nonoperative periods of medical procedures. The videos 3110 can each include frames of images (e.g., images 250b, 255b, 325a-e, 330a-e, 335a-e, and so on).

Applicant recognizes that by providing the information of a plurality of medical procedures using the hierarchical structure 3100, users having different roles such as a medical staff, a consultant, and a student can efficiently and expediently arrive at desired detailed information (e.g., the first level, the second level, and the third level) from more general information (e.g., the fourth level and the fifth level). A user can quickly and intuitively become acclimated to navigating the user interface with the hierarchical structure 3100 without a steep learning curve to discover the information of a plurality of medical procedures, regardless of the role of the user. For example, to arrive at the desired individual case data 3130, the user only needs to provide the user input two times. To arrive at the desired timelines 3120 and videos 3110, the user only needs to provide the user input three and four times, respectively.

The hierarchical structure 3100 can facilitate optimization of OR workflow. For example, workflow efficiency can be measured using nonoperative temporal metrics that are compared within each hospital, each OR, each care team, each procedure, and so on and compared/contrasted against peers and historical data. The comparisons within the same hospital are often evaluated for similar categories to ensure fairness. For instance, two surgical teams of the same hospital that have operated the same type of procedures can be compared/contrasted to discover efficiencies and inefficiencies of both teams. The hierarchical structure 3100 can allow discovery of information that can improve performance of one team by exemplifying decisions of another team and to improve operation consistency of multiple teams within the hospital.

The hierarchical structure 3100 can facilitate optimization for room layout. For example, room layout can be measured through comparisons between ORs within a hospital or different hospitals, as well as against a recommended room layout. As noted herein, the theater-wide sensors are used to map the 3D location of equipment and personnel within an OR. The hierarchical structure 3100 can allow discovery of information that can improve room layout by exemplifying another room layout or a recommended room layout.

The hierarchical structure 3100 can facilitate optimization of care team size. The number of medical staff members involved in each phase, task, or procedure of nonoperative time can be measured using computer vision detection algorithm as described herein. Minimum, average, and maximum number of medical staff members are then compared/contrasted against other care teams, against recommended number of medical staff members for completion of tasks, and against historical data to provide insights. The hierarchical structure 3100 can allow discovery of information that can improve care team size by exemplifying the sizes of other care teams, a recommended care team size, and so on.

The hierarchical structure 3100 can facilitate optimization of access management and scheduling. Access management performance can be measured by comparing the metrics and statistics against known guidelines and best practices. For example, the nonoperative metrics for a period of time (e.g., days) with three or more procedures per day can be compared/contrasted with the nonoperative metrics for another period of time (having the same length) in the same OR with a number of procedures fewer than three. Often the care teams are more efficient when they operate more of the same type of procedures within a period of time. The hierarchical structure 3100 can allow discovery of information that can improve performance of a care team within current competency and without requiring further training.

Accordingly, the hierarchical structure 3100 enables the user to seamlessly interact with information presented within a particular level of the hierarchical structure 3100 to obtain additional and/or a more in-depth view of the data in another level of the hierarchical structure 3100. A user can interact with a user interface element presenting information of a plurality of medical procedures to arrive at a more detailed view of the information of a plurality of medical procedures.

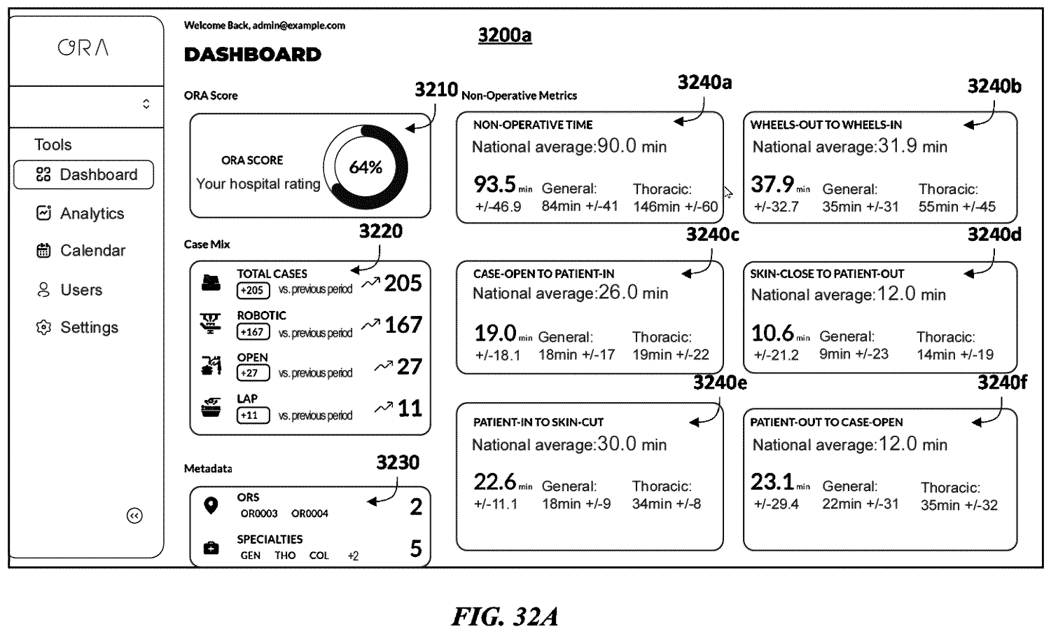

FIG. 32A is a screenshot illustrating an example user interface 3200a showing highlights 3150 of the information of a plurality of medical procedures, according to some examples. The user interface 3200a is an example of the fifth level of the user interface. As shown, the user interface 3200a includes selected portions of the aggregated data 3140, including the ORA score 3210, case mix information 3220, the metadata 3230, and nonoperative metrics such as nonoperative time 3240a, wheals-out to wheals-in time 3240b, case-open to patient-in time 3240c, skin-close to patient-out time 3240d, patient-in to skin-cut 3240e, patient-out to case-open 3240f. Each of the ORA score 3210, the mix information 3220, the metadata 3230, and the nonoperative metrics 3240a-f can be configured as user interactive elements such that, in response to being selected by the user, can trigger displaying of additional aggregated data 3140.

The ORA score 3210 can be an aggregated metric value determined for a hospital. The case mix information 3220 can include aggregated statistics for the hospital, including the total number of cases (medical procedures), cases in which a robotic system is involved, open cases, and lap cases. The metadata 3230 includes identifying information of the ORs, specialties, procedures, surgeons, and so on. The nonoperative metrics 3240a-f can be aggregated metrics (e.g., average) across different ORs in the hospital or by different staff members in the hospital for various intervals (e.g., tasks or phases). For example, the nonoperative metrics 3240a-f at the highlight level can include an average of a particular nonoperative metric across all cases, procedures, or medical environments from a hospital. The user interface 3200a for another hospital displays information for that hospital. Another example of the fifth level of the user interface containing the highlights 3150 is shown in FIG. 26.

FIG. 32B is a screenshot illustrating an example user interface 3200b showing highlights 3150 (e.g., trends) of the information of a plurality of medical procedures, according to some examples. The user interface 3200b is an example of the fifth level of the user interface. As shown, the user interface 3200b includes selected portions of the aggregated data 3140, including the ORA score 3510, case mix information 3260, the metadata 3270, and nonoperative metrics such as nonoperative time 3280a, wheals-out to wheals-in time 3280b, case-open to patient-in time 3280c, skin-close to patient-out time 3280d, patient-in to skin-cut 3280e, patient-out to case-open 3280f. Each of the ORA score 3250, the mix information 3260, the metadata 3270, and the nonoperative metrics 3280a-f can be configured as user interactive elements such that, in response to being selected by the user, can trigger displaying of additional aggregated data 3140.

The ORA score 3250 can be an aggregated metric value determined for a hospital. The case mix information 3260 can include aggregated statistics for the hospital, including the total number of cases (medical procedures), cases in which a robotic system is involved, open cases, and lap cases. The metadata 3270 includes identifying information of the ORs, specialties, procedures, surgeons, and so on. The nonoperative metrics 3280a-f can be aggregated metrics (e.g., average) across different ORs in the hospital or by different staff members in the hospital for various intervals (e.g., tasks or phases). For example, the nonoperative metrics 3280a-f at the highlight level can include an average of a particular nonoperative metric across all cases, procedures, or medical environments from a hospital. The user interface 3200b for another hospital displays information for that hospital.

Trends in information for medical procedures can be more revealing in some situations than outliers given that outliers may be caused by special circumstances which may not be reproducible in other instances. Trends can be displayed to a user at the highest level using indicators corresponding to positive or negative trends. In some examples, a positive trend can be represented using a first color (e.g., green) or a first graphical element (e.g., upward arrow), and a negative trend can be represented using a second color (e.g., red) or a second graphical element (e.g., downward arrow). For instance, metric 3280b for the wheels-out to next wheels-in durations are represented using a red number, indicating that there is a trend that this metric 3280b has been consistently decreasing, meaning that it is trending in the positive direction) over a period of time (e.g., a month). In addition, the case mix information 3260 such as the total number of cases, robotic cases, open cases, and lap cases are each shown with an upward arrow, indicating a positive trend upwards for a particular hospital.

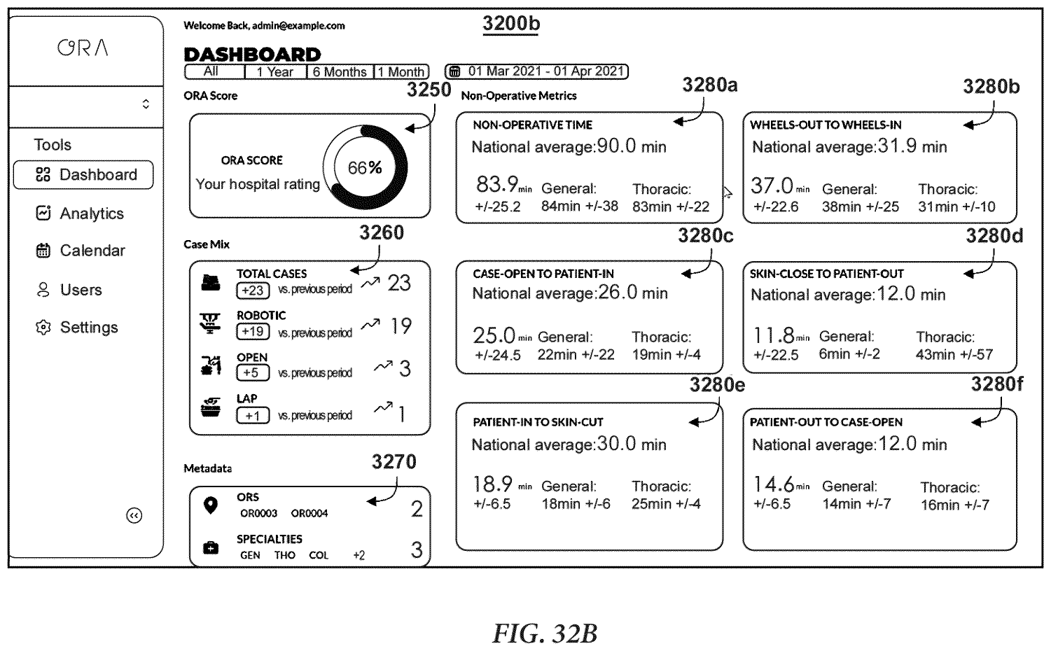

FIG. 33 is a screenshot illustrating an example user interface 3300 showing aggregated data 3140 of the information of a plurality of medical procedures, according to some examples. The user interface 3300 is an example of the fourth level of the user interface. In some examples, the user interface 3300 is displayed in response to the user selecting any one of the user interactive elements for the nonoperative metrics 3240a-f. As shown, the user interface 3300 includes metrics 3310 for nonoperative time, wheals-out to wheals-in time, case-open to patient-in time, skin-close to patient-out time, patient-in to skin-cut, patient-out to case-open, determined with respect to the hospital, an average of multiple hospitals (e.g., hospitals in the nation), and top 20% of the multiple hospitals. The metrics 3310 are shown in bar graphs.

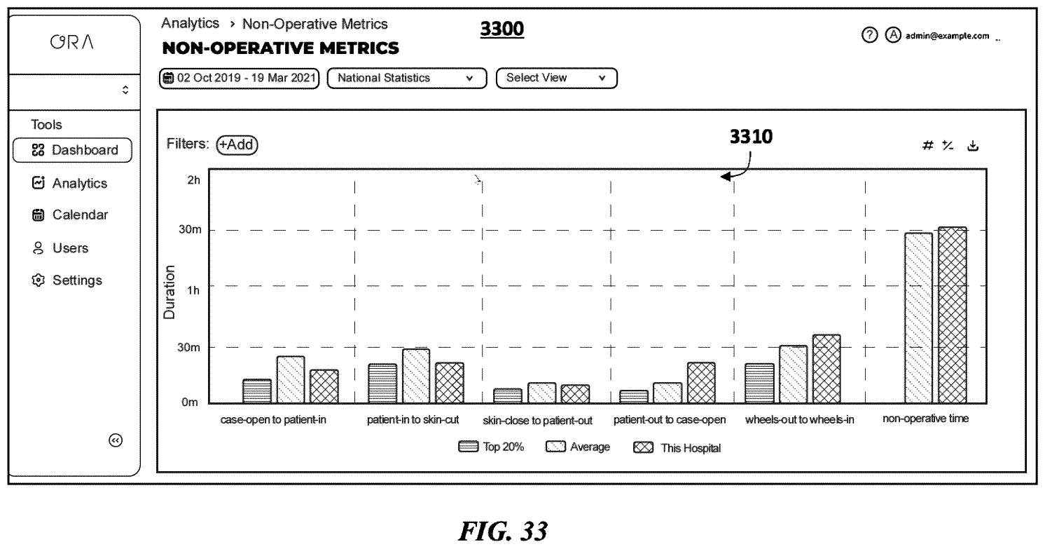

FIG. 34 is a screenshot illustrating an example user interface 3400 showing aggregated data 3140 of the information of a plurality of medical procedures, according to some examples. The user interface 3400 is an example of the fourth level of the user interface. In some examples, the user interface 3400 is displayed in response to the user selecting a user interactive element for the case mix information 3220. As shown, the user interface 3400 includes detailed case mix information 3410 including the number of different medical procedures (e.g., inguinal hernia repair, cholecystectomy, umbilical hernia repair, paraoesophageal hernia repair, hysterectomy, and other procedures) performed within a hospital within a predetermined period of time (e.g., a year). The case mix information 3410 is shown in a pie graph.

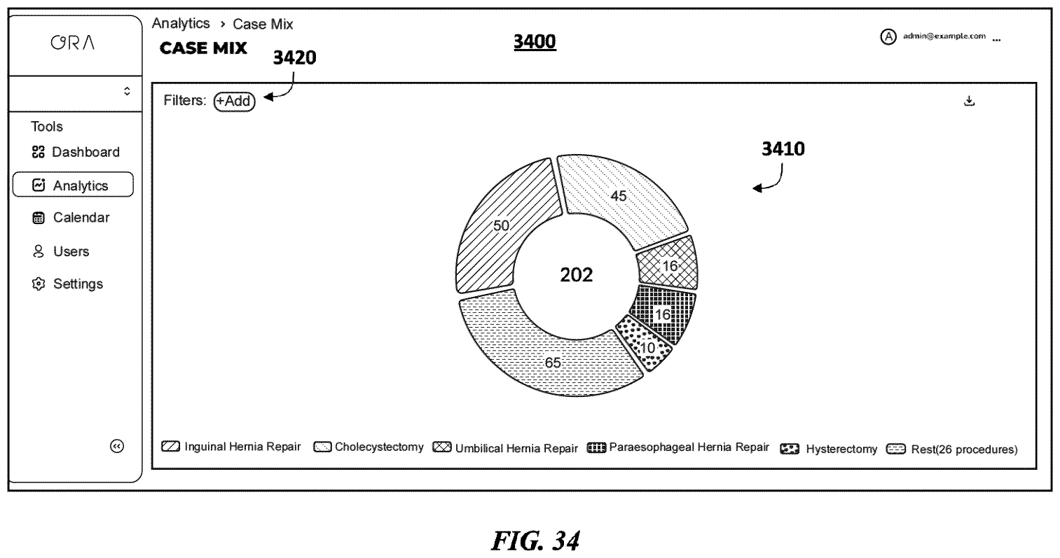

FIG. 35 is a screenshot illustrating an example user interface 3500 showing aggregated data 3140 of the information of a plurality of medical procedures (e.g., modality related information), according to some examples. The user interface 3500 is an example of the fourth level of the user interface. In some examples, the user interface 3500 is displayed in response to the user selecting a user interactive element for the case mix information 3220. As shown, the user interface 3500 includes detailed case mix information 3510 including the number of different procedure modalities (e.g., robotic, open, lap, and so on) performed within a hospital within a period of time. The case mix information 3510 is shown in a bar graph.

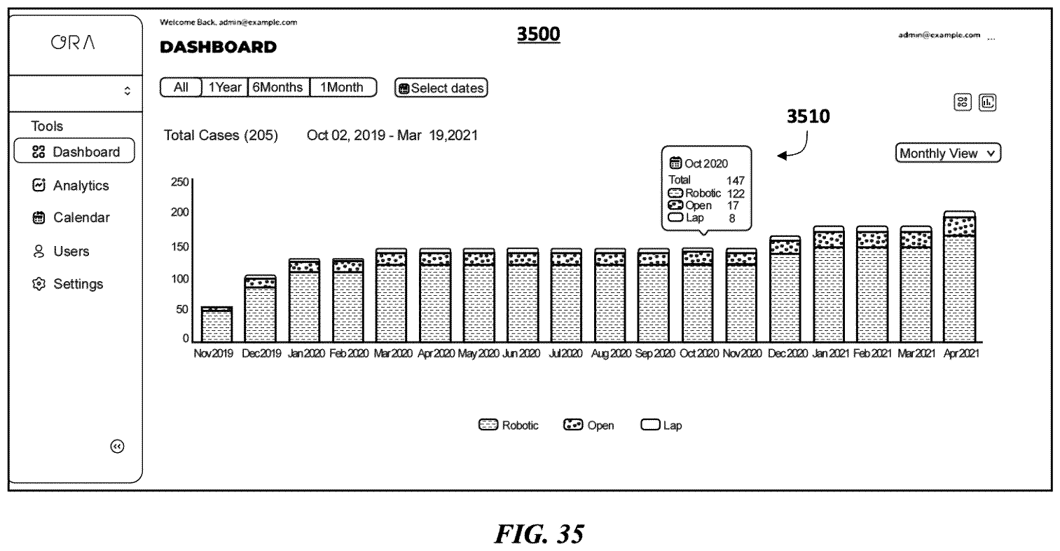

FIG. 36 is a screenshot illustrating an example user interface 3600 showing individual case data 3130 of the information of a plurality of medical procedures, according to some examples. The user interface 3600 is an example of the third level of the user interface. In some examples, the user interface 3600 is displayed in response to the user selecting a user interactive element for an OR in the interface 3500. As shown, the user interface 3600 includes individual case data 3610 such as non-operative metrics (e.g., compound non-operative metrics) for different cases or procedures (e.g., case 1-10), each of which includes multiple phases (e.g., case-open to patient-in, patient-in to skin-cut, and so on). For a given case, the individual case data 3610 includes metadata 3620 such as a case ID, a date, a day of the week, an OR ID, a case number, a procedure name, a specialty of the OR or surgeon, surgeon name, modality (e.g., robotic or non-robotic), scheduled time, and so on. The user interface 3600 can include timelines having various elements. The length of each element displayed corresponds to a length of the duration of each phase. Each element displayed for a phase can be configured as a user interactive element such that, in response to being selected, triggers the display of another user interface showing a video 3110 corresponding to the phase. The timelines in the user interface 3600 corresponds to compound non-operative metrics instead of atomic activities detected by the vision algorithm as described.

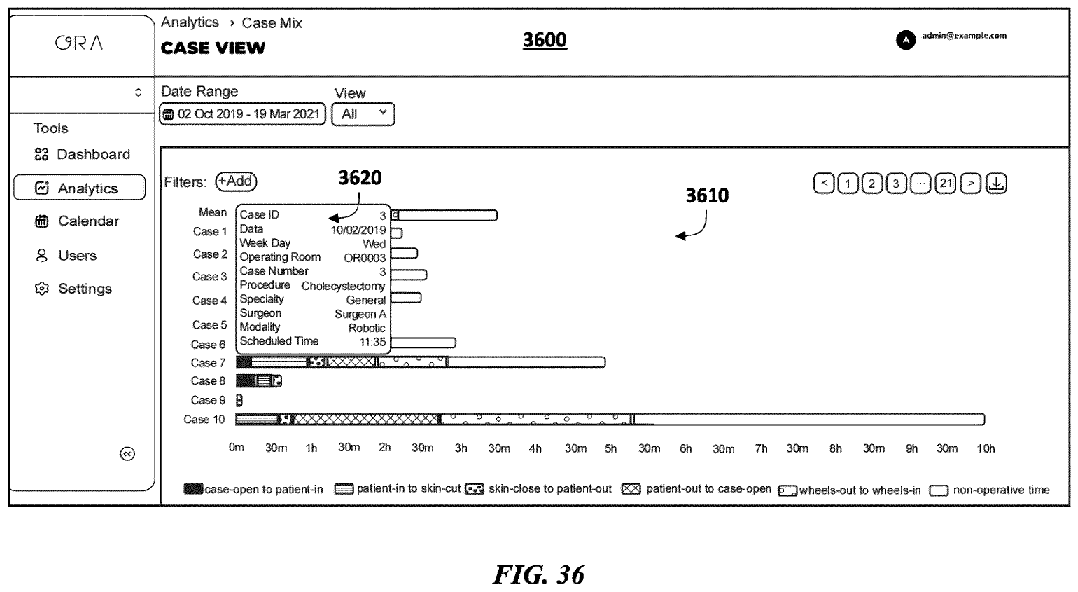

FIG. 37 is a screenshot illustrating an example user interface 3700 showing timelines 3120 of the information of a plurality of medical procedures, according to some examples. The user interface 3700 is an example of the second level of the user interface. In some examples, the user interface 3700 is displayed in response to the user selecting a user interactive element for a procedure shown in the interface 3600. As shown, the user interface 3700 includes timelines 3710 (examples of the timelines 3120) of different phases (e.g., turnover, main phase, patient-prep phase, robotic phase, and so on) and their respective constituent tasks (e.g., turnover, sterile preparation, patent-in, patient preparation, and so on). The length of each element displayed corresponds to a length of the duration of each task. Each element displayed for a phase or a task can be configured as a user interactive element such that, in response to being selected, triggers the display of another user interface showing a video 3110 corresponding to the phase or the task.

In some embodiments, the system can provide interactive filters to allow a user to interact with data manually by removing at least one option or a portion of the information for a plurality of medical procedures from all options or all information for a plurality of medical procedures. Filter options are sorted by importance or relevance based on one or more of case volume, time, location, surgeon, and so on. For example, options and information for a plurality of medical procedures that are more important or relevant are displayed more prominently (e.g., above, in larger font size, in brighter color, and so on) than less important options and information for a plurality of medical procedures. Certain inapplicable and less important options and information for a plurality of medical procedures are disabled from being displayed on the user interface.

Options and information of a plurality of medical procedures for operations, hospitals, ORs, surgeons, and so on with more case volume are more important than options and information of a plurality of medical procedures for operations, hospitals, ORs, surgeons, and so on with less case volume, including for interactive filters and suggested views as described herein. This is because improvements made in categories with higher case volume have a greater impact in overall efficiency improvement. For a given type of data (e.g., for a given surgeon, hospital, etc.) that has low case volume, there may not be sufficient quantity of data to be analyzed to yield meaningful results such as trends and averages for consideration.

Options and information of a plurality of medical procedures (such as theater-wide data, statistics, metrics, and so on) collected or determined later in time can be more important than options and information of a plurality of medical procedures collected or determined earlier in time in some examples. This is because recent data can be more relevant to the current state of operations of surgeons, hospitals, etc. that may continuously improve using the methods described herein.

Options and information of a plurality of medical procedures collected at or determined for a hospital or an OR associated with the particular user are more important than options and information of a plurality of medical procedures collected at or determined for another location. For example, options and information of a plurality of medical procedures collected at or determined for a hospital or an OR is determined to be important in response to determining that at least one of a location of the user device of the user is currently at the hospital or OR, the user has a role that is associated with the hospital or OR. For example, the user can be a surgeon who operated in the OR, an administrator at the hospital, or a student or consultant assigned to study or evaluate procedures at the hospital or OR. For example, options and information of a plurality of medical procedures collected at or determined for procedure is determined to be important in response to determining that the user participated in the procedure (e.g., as a surgeon, robotic system operator, support staff, and so on) or is assigned to study the procedure (e.g., as a consultant assigned to evaluate the procedure).

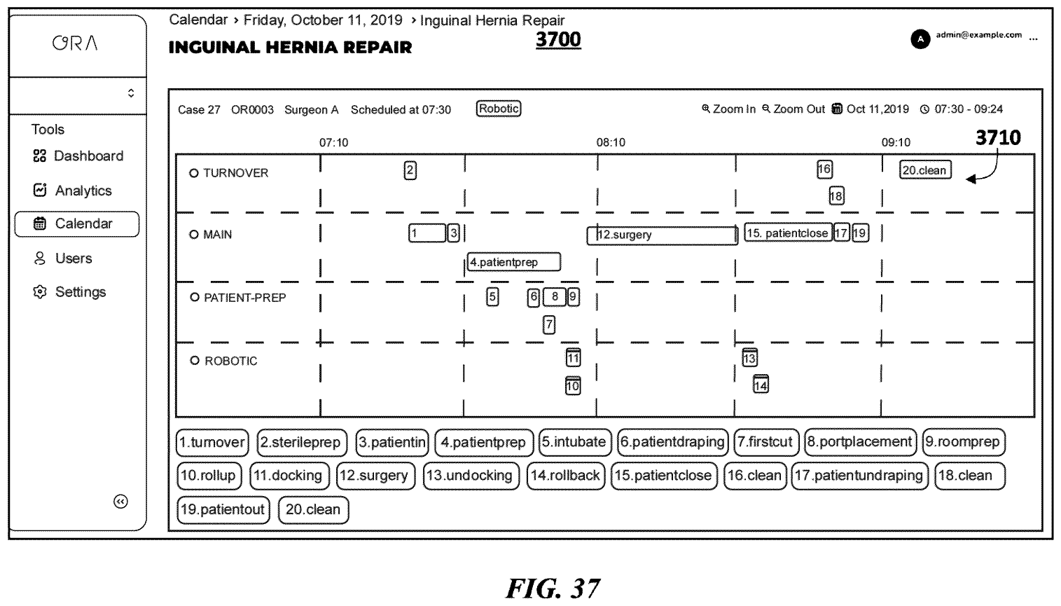

FIG. 38 is a screenshot illustrating an example user interface 3800 showing interactive filter 3810, according to some examples. The user interface 3800 is an example fourth level interface containing the aggregated data 3140. The interactive filter 3810 can be displayed in response to selecting a user interactive element corresponding to the interactive filter 3810 in the user interface 3300. As shown, the interactive filter 3810 includes filters for general classifications of medical procedures (e.g., general, thoracic, colorectal, and so on), surgeons, and specific types of medical procedures (e.g., inguinal hernia repair, cholecystectomy, and so on). The filter options can be sorted or arranged according to importance in the manner described.

Filter options can be sorted based on importance and relevance in the manner described for additional instances as the user applies a filter. For example, in response to the user selecting a filter corresponding to a particular surgeon (e.g., Dr. Daskalakis), the general classifications and the procedure types can be filtered further based on the case volume that the selected surgeon has performed. For example, in response to determining that the selected surgeon has performed medical procedures for a general classification or procedure type is above a threshold (e.g., 0, 1, 2, etc.), that general classification (e.g., general and colorectal) or procedure type (e.g., inguinal hernia repair, cholecystectomy) is determined to be important and is displayed prominently (e.g., in dark text color) as compared to other general classifications or procedure types determined to be less important. Accordingly, a user can easily discover which procedures a surgeon has operated by simply selecting on the surgeon's name in a surgeons' panel and inspecting the remaining options in procedures section.

In another example, in response to the user selecting a filter corresponding to a particular type of medical procedure, additional filter options related to surgeons can be sorted according to a case volume for that selected medical procedure of each surgeon. Therefore, interactive filters allow a user to apply its own filters while providing recommended filters based on importance and relevance, thus improving user experience of savvy users who prefer to interact with information of a plurality of medical procedures manually.

In the example shown in FIG. 32A and FIG. 32B, the ORA score 3210/3250, the case mix information 3220/3260, the metadata 3230/3270, and nonoperative metrics 3240a-f/3280a-f shown are aggregated data computed for and across the hospital (e.g., for all medical staff members of the hospital, for all ORs in the hospital, for all procedures of the hospital, and so on), in response to determining at least one of the user has a role associated with the hospital, the user has a role of administrator or consultant, the user is affiliated with the hospital, the user has previously inputted a preference for suggested views for the hospital, the user has previously participated in a procedure in the hospital, the user is currently located in the hospital, and so on. The location of the user can be determined by the location (e.g., global positioning system (GPS) coordinates, network connections, and so on) of a user device running the application on which the user interface is displayed.

In some embodiments, to suggest displaying information of a plurality of medical procedures, the system performs multiple layers of analysis to suggest views that likely lead to insightful discoveries. The suggestion can be determined based on importance of the data, which in some cases can be estimated based on volume. For instance, surgeons with larger case volumes of a certain procedure type can be cross-referenced, where the procedure type may also have large volumes. In some examples, surgeons that share common procedures are suggested for comparison. Such comparison is fair given that the surgeons are compared for the same type of procedure. Any statistically significant difference in the aggregate data 3140 can cause a difference in performance of the care teams that work with those surgeons. Such suggested views include top procedures (e.g., procedures with the largest number of case volumes), top surgeons (e.g., surgeons with the largest number of case volumes), procedures of the top surgeons (ranked according to case volumes), and so on. The statistics and the metrics of the top procedures, top surgeons, and the procedures of those surgeons can be suggested to be cross referenced for evaluation of efficiencies and inefficiencies. In some examples, filters and options in a user interface can be provided to yield the desired aggregate statistics for comparison. Such features can significantly streamline the discovery process and smoothens the learning curve for novice users.

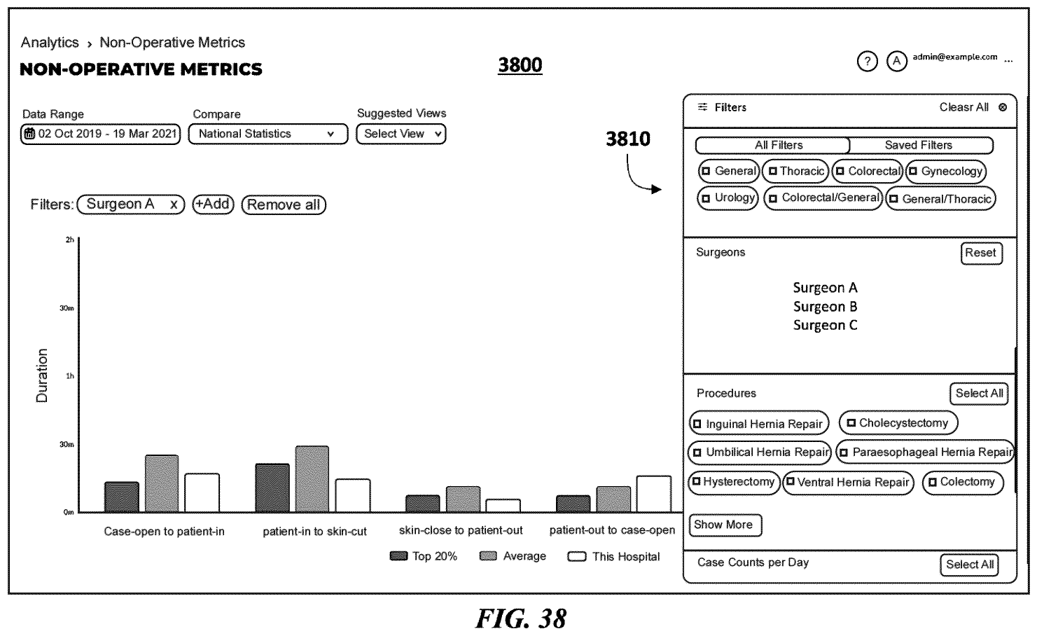

FIG. 39 is a screenshot illustrating an example user interface 3900 displayed in response to selection using an interactive filter, according to some examples. The user interface 3900 is an example fourth level interface containing the aggregated data 3140. The user interface 3900 can be displayed in response to selecting a user interactive element 3420 corresponding to the interactive filter in the user interface 3400. As shown, the interactive filter applies a filter for a particular OR as selected by the user. The detailed case mix information 3410 is modified to the case mix information 3920.

In some embodiments, automated discovery and recommended view features can be provided to customize the information presented in each level of the hierarchical structure 3100 to reduce learning curve of the user by presenting information that is most relevant to the user. This allows a new user without a substantial amount of experience navigating the user interface to discovery relevant information expeditiously. In some examples, different recommended information can be displayed for different users, different hospitals, different ORs, different procedures, and so on, allowing users to discover relevant information via different pathways. In some examples, the procedure-relate information displayed in any level of the hierarchical structure 3100 can be recommended based on the user, the hospital, OR, procedure, time, and so on.

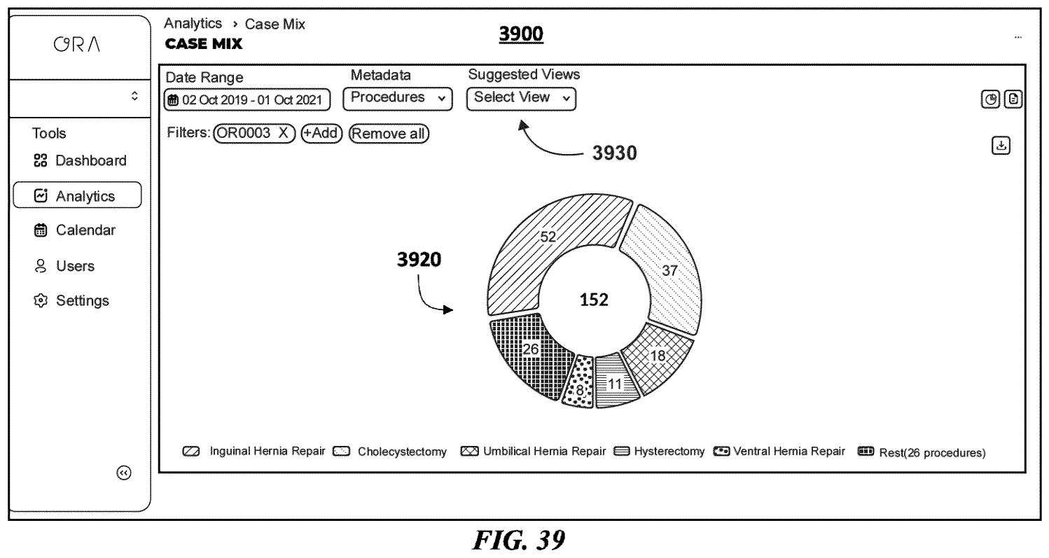

FIG. 40 is a screenshot illustrating an example user interface 4000 showing suggested view element 4010, according to some examples. The user interface 4000 is an example fourth level interface containing the aggregated data 3140. The suggested view element 4010 can be displayed in response to selecting a user interactive element 4030 corresponding to the suggested view element 4010 in the user interface 4000. As shown, in response to selecting the suggested view element 4010, various suggested options 4020 recommended based on the surgeons are displayed. In other words, for a given hospital, the suggested options 4020 can include options for the procedure-relate information relevant to the OR, including specialties in the OR, procedures performed in the OR, procedures performed by surgeons in the OR, and so on. Such suggested views can be predefined for the hospital.

In response to selecting the user interactive element for specialties in OR, a list of medical procedure classifications or types performed in the OR having a case volume above a threshold can be shown. The list can be sorted according to case volume, with medical procedure classifications or types having a greater volume being displayed more prominently than (e.g., above) medical procedure classifications or types having a lesser volume. In response to selecting the user interactive element for procedures in OR, a list of medical procedures performed in the OR can be shown. The list can be sorted according to time, with medical procedures performed at a later time being displayed more prominently than (e.g., above) medical procedures performed at an earlier time. In response to selecting the user interactive element for procedures by surgeon, a list of surgeons who performed medical procedures in the OR can be shown. The list can be sorted according to case volume, with surgeon performing a greater volume of procedures within the OR being displayed more prominently than (e.g., above) surgeon performing a lesser volume of procedures within the OR. Suggested views for an OR, a hospital, a hospital group, a surgeon, a medical procedure classification, a medical procedure type, and so on can be implemented in the manner described. The data (e.g., case volume) used in the sorting of the options can be defined using metadata such as a date range.

According to some embodiments, a fifth level (e.g., a top or highest level of information) or highlights 3150 as well as interfaces of other levels present a customized user interface of information of a plurality of medical procedures generated by the system. Such user interface is customized or individualized based on information associated with the user including for example an identity of the user, a role of the user (e.g., a surgeon, an administrator, a consultant, etc.), entity affiliation(s) of the user, preferences of the user, procedures performed by the user (e.g., types of procedures, number of procedures, etc.), a location of the user, and the like.

According to some embodiments, a fifth level or another level of the user interface can be customized or individualized based on information of a plurality of medical procedures computed by the system that are relevant to the particular user. In one example, the fifth level or another level of the user interface for a user who is a surgeon can be individualized based on information of a plurality of medical procedures computed for procedures performed by the surgeon. For instance, the fifth level of the user interface can present comparisons between the information of a plurality of medical procedures (e.g., first metrics) computed for the surgeon and information of a plurality of medical procedures (e.g., second metrics) for the same or similar procedures computed for other surgeons.

In another example, the fifth level or another level of the user interface for a user who is an administrator of a hospital can be individualized based on information of a plurality of medical procedures computed for procedures performed in the hospital. In yet another example, the fifth level or another level of the user interface for a user who is an administrator of a health network can be individualized based on information of a plurality of medical procedures computed for procedures performed at the individual facilities (e.g., multiple hospitals or a hospital group) of the health network.

In one aspect, the fifth level or another level of the user interface of the user interface can be further customized or individualized based on comparison between the information of a plurality of medical procedures relevant to the user to information of a plurality of medical procedures computed for other users, entities, and/or a group of users and/or entities. In an example in which particular information of a plurality of medical procedures related to the user is an outlier or a trend (which may be an indication of efficiency or inefficiency), the user interface features or elements related to the particular procedure can be presented more prominently within the fifth level (or another level) of the user interface. In this manner, the system is configured to intelligently present, within the first level of the hierarchical UI, information that may be most relevant to the user in evaluating and discovering efficiencies and/or inefficiencies in procedures.

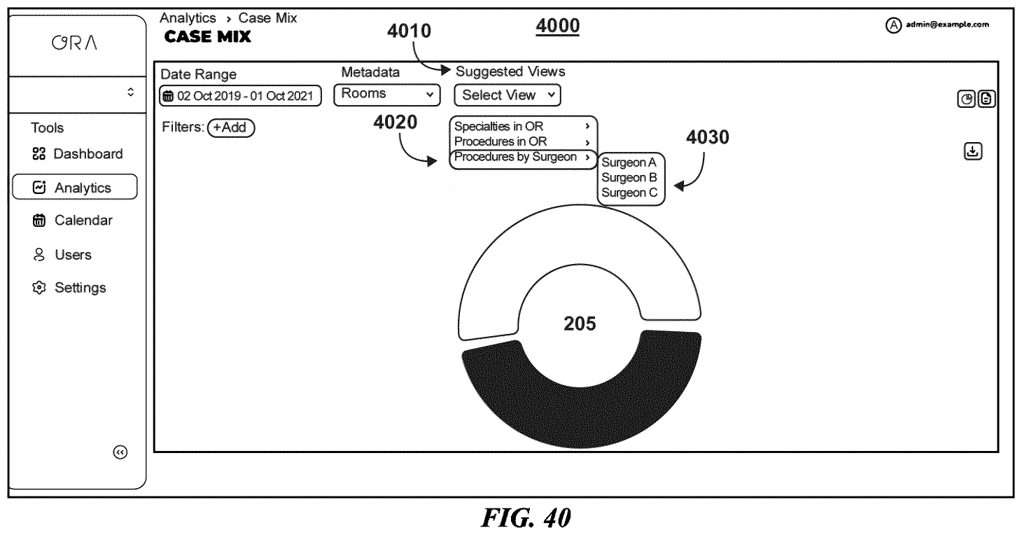

FIG. 41 is a screenshot illustrating an example user interface 4100 showing nonoperative metrics 4110 for multiple surgeons and multiple phases/tasks, according to some examples. The user interface 4100 is an example fourth level interface containing the aggregated data 3140. For example nonoperative metric of average staff member count for different surgeons and for different phases are shown in the same interface 4100. Each phase includes intervals each denoting a task. By interacting (e.g., mouse over) with a user interactive element corresponding to a particular phase or task as shown, more detailed information (e.g., maximum, mean, minimum, standard deviation, and sample sizes of the staff member count) are displayed as the individual case data 3130.

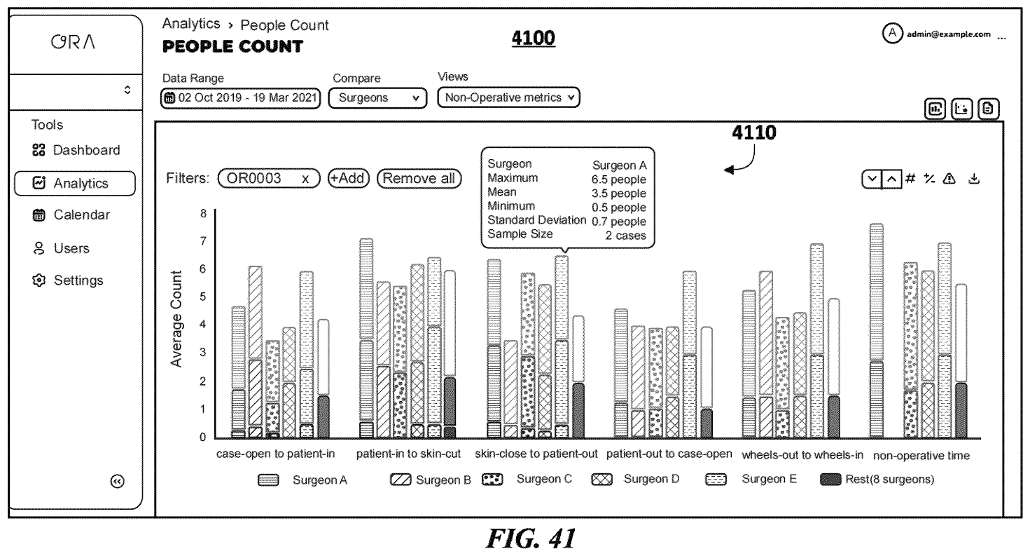

FIG. 42 is a screenshot illustrating an example user interface 4200 showing nonoperative metrics 4210 for multiple surgeons for a given procedure (inguinal hernia repair), according to some examples. The user interface 4200 is an example fourth level interface containing the aggregated data 3140. For example, the nonoperative metrics 4210 of durations for six different phases (e.g., case-open to patient-in, patient-in to skin-cut, etc.) by two different surgeons are shown in the same interface 4200. The user interface 4200 is displayed in response to a user selecting a suggested view to compare two surgeons (e.g., two top surgeons by case volume) for the medical procedure. By interacting with a user interactive element corresponding to a particular phase for a particular surgeon, more detailed information are displayed as the individual case data 3130.

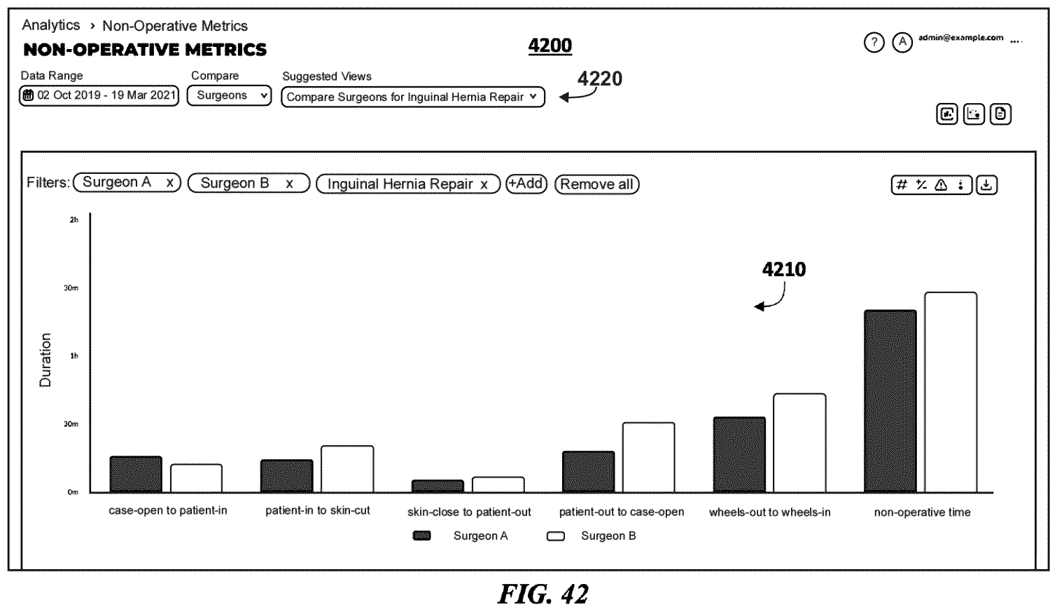

FIG. 43 is a schematic block diagram depicting a method 4300 for providing medical information of a plurality of medical procedures using a hierarchical structure (e.g., the structure 3100), as may be implemented in some embodiments. The method 4300 can be performed using the system (e.g., the systems 190a, 190b, 450b, and 3000).

At 4310, the system receives a plurality of data streams of information of a plurality of medical procedures of a plurality of medical procedures. The information of a plurality of medical procedures includes case metadata of the plurality of medical procedures, a timeline defined by phase data relating to a plurality of phases identified for each of the plurality of medical procedures and task data relating to a plurality of tasks identified within each of the plurality of phases, and three-dimensional point cloud data for each of the plurality of medical procedures during at least portions of the plurality of phases and the plurality of tasks within each of the phases. In some examples, the phase data includes one or more of a start time, an end time, a length of a time duration (e.g., the time duration between the start time and the end time), and a name, description, class, category, or assignment of a phase (e.g., “post-surgery,” “turnover,” “pre-surgery,” and “surgery,” etc.). In some examples, the task data includes one or more of a start time, an end time, a length of a time duration (e.g., the time duration between the start time and the end time), and a name, description, class, category, or assignment of a task (e.g., “port placement,” “rollup,” etc.).

At 4320, the system provides for display at least a portion of the information of a plurality of medical procedures using a hierarchical user interface structure. The hierarchical structure includes a first level of a user interface to display, based at least on the three-dimensional point cloud data, a three-dimensional point cloud representation of a task of a phase selected from a timeline of a second level of user interface. The hierarchical structure includes the second level of the user interface to display the timeline and a portion of the case metadata associated with the timeline.

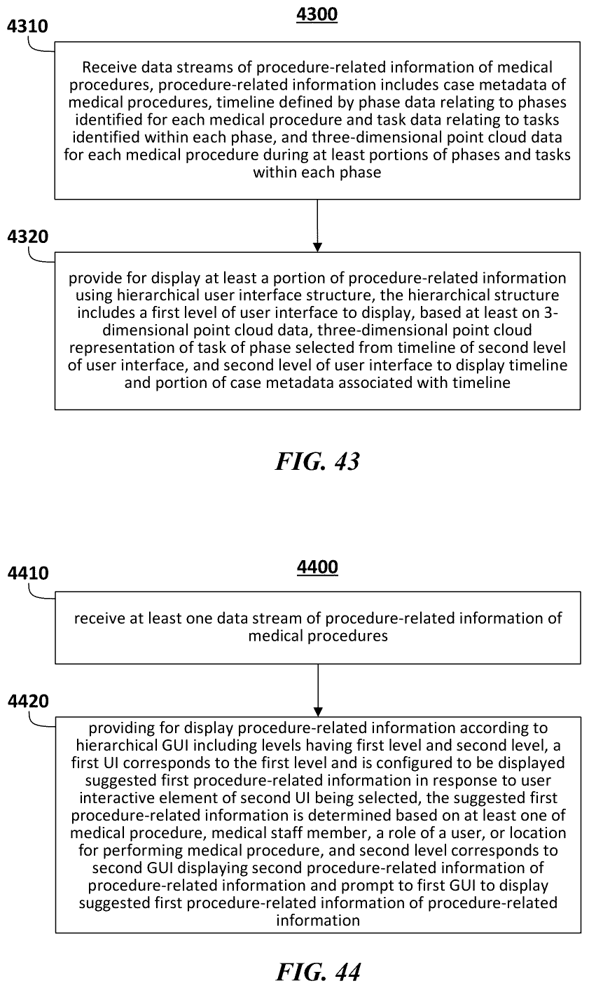

FIG. 44 is a schematic block diagram depicting a method 4400 for providing medical information of a plurality of medical procedures using a hierarchical structure (e.g., the structure 3100), as may be implemented in some embodiments. The method 4400 can be performed using the system (e.g., the systems 190a, 190b, 450b, and 3000).

At 4410, the system receives at least one data stream of information of a plurality of medical procedures of a plurality of medical procedures. At 4420, the system provides for display the information of a plurality of medical procedures according to a hierarchical GUI including a plurality of levels having a first level and a second level. A first GUI corresponds to the first level and is configured to display suggested first information of a plurality of medical procedures in response to a user interactive element of a second GUI being selected. The suggested first information of a plurality of medical procedures is determined based on at least one of a medical procedure, a medical staff member, a role of a user, or a location for performing the medical procedure. The second level corresponds to the second GUI displaying second information of a plurality of medical procedures of the information of a plurality of medical procedures and a prompt to the first GUI to display the suggested first information of a plurality of medical procedures of the information of a plurality of medical procedures.

In some embodiments, the information of a plurality of medical procedures displayed in an interface of each of the levels 3110, 3120, 3130, 3140, 3150, and 3160 can be selected based on a role of a user. That is, at least some information displayed in an interface of each of the levels 3110, 3120, 3130, 3140, 3150, and 3160 is associated with or mapped to a role of the user. In some examples, examples of the roles of a user include a surgeon, a medical staff member, a hospital administrator, a hospital group administrator, a cross-institution administrator, a consultant, a student, and so on. In some examples, a role of a user can be defined in a user profile according to user input, e.g., a user can input or select a role and associate that role with the user's credentials (e.g., name, password, ID, and so on). In some examples, the role of the user can be defined based on a location of a user device running the application on which the user interface is displaced. For instance, in response to determining that the GPS coordinate of a user device operated by the user is within an area defining a hospital, the role of the user can be determined to be a surgeon, a medical staff member, or a hospital administrator in that hospital.

In some examples, the role of a user can be mapped to types of information to be displayed in an interface of each of the levels 3110, 3120, 3130, 3140, 3150, and 3160. In the examples in which the user has a role of a surgeon or a medical staff member, the information displayed at each of the levels 3110, 3120, 3130, 3140, 3150, and 3160 can include information for medical procedures performed by the user, hospitals or ORs in which the user has performed medical procedures, specialties of the user, and so on. In the examples in which the user has a role of a hospital administrator (who manages a hospital), the information displayed at each of the levels 3110, 3120, 3130, 3140, 3150, and 3160 can include information for medical procedures performed in that hospital, surgeons and medical staff members at that hospital, and so on. In the examples in which the user has a role of a hospital group administrator or a cross-institution administrator (who manages multiple hospitals), the information displayed at each of the levels 3110, 3120, 3130, 3140, 3150, and 3160 can include information for medical procedures performed in those hospitals, surgeons and medical staff members at those hospitals, and so on. In the examples in which the user has a role of a consultant or a student, the information displayed at each of the levels 3110, 3120, 3130, 3140, 3150, and 3160 can include information for medical procedures to be analyzed or studied by the user, hospitals to be analyzed or studied by the user, surgeons and medical staff members to be analyzed or studied by the user, and so on.

In some examples, in FIG. 32A and FIG. 32B, the ORA score 3210/3250, the case mix information 3220/3260, the metadata 3230/3270, and nonoperative metrics 3240a-f/3280a-f shown are aggregated data computed for and across a hospital (e.g., for all medical staff members of the hospital, for all ORs in the hospital, for all procedures of the hospital, and so on), in response to determining that a user has a role of a surgeon, a medical staff member, or a hospital administrator. In some examples, in FIG. 32A and FIG. 32B, the ORA score 3210/3250, the case mix information 3220/3260, the metadata 3230/3270, and nonoperative metrics 3240a-f/3280a-f shown are aggregated data computed for and across multiple hospitals (e.g., for all medical staff members of the hospitals, for all ORs in the hospitals, for all procedures of the hospitals, and so on), in response to determining that a user has a role of a hospital group administrator, a cross-institution administrator, a consultant, a student.

In some examples, in FIG. 33, the metrics 3310 shown are aggregated data computed for and across multiple hospitals in a nation (e.g., for all medical staff members of the hospitals, for all ORs in the hospitals, for all procedures of the hospitals, and so on), in response to determining that a user has a role of a hospital group administrator, a cross-institution administrator, a consultant, a student.

In some examples, in FIG. 34, the case mix information 3410 shown is aggregated data computed for and across a hospital (e.g., for all medical staff members of the hospital, for all ORs in the hospital, for all procedures of the hospital, and so on), in response to determining that a user has a role of a hospital administrator. In some examples, in FIG. 34, the case mix information 3410 shown is aggregated data computed for and across procedures performed by a surgeon or a medical staff, in response to determining that a user has a role of the surgeon or the medical staff member.

In some examples, in FIG. 35, the case mix information 3510 shown is aggregated data computed for and across a hospital (e.g., for all medical staff members of the hospital, for all ORs in the hospital, for all procedures of the hospital, and so on), in response to determining that a user has a role of a hospital administrator. In some examples, in FIG. 35, the case mix information 3510 shown is aggregated data computed for and across procedures performed by a surgeon or a medical staff, in response to determining that a user has a role of the surgeon or the medical staff member.

In some examples, in FIG. 36, the individual case data 3610 of different cases is for a hospital (e.g., for all medical staff members of the hospital, for all ORs in the hospital, for all procedures of the hospital, and so on), in response to determining that a user has a role of a hospital administrator. In some examples, in FIG. 36, the individual case data 3610 of different cases is for a surgeon or a medical staff member, in response to determining that a user has a role of the surgeon or the medical staff member.

In some examples, in FIG. 37, the timelines 3710 for a procedure performed by a surgeon is displayed, in response to determining that a user has a role of a surgeon, a medical staff member, hospital administrator.

In some examples, the system selects at least the portion of the information of the plurality of medical procedures according to a role of a user interacting with the user interface. At least the portion of the information is mapped to the role of the user. In some examples, the role of the user comprises at least one of a surgeon, a medical staff member, a hospital administrator, a hospital group administrator, a cross-institution administrator, a consultant, or a student. In some examples, the role of the surgeon or the medical staff member is mapped to at least the portion of the information of the plurality of medical procedures performed by a respective one of the surgeon or the medical staff member. In some examples, the role of the hospital administrator is mapped to at least the portion of the information of the plurality of medical procedures performed in a hospital. In some examples, the role of the hospital group administrator, the cross-institution administrator, the consultant, or the student is mapped to at least the portion of the information of the plurality of medical procedures performed in a plurality of hospitals.

### Remarks

The drawings and description herein are illustrative. Consequently, neither the description nor the drawings should be construed so as to limit the disclosure. For example, titles or subtitles have been provided simply for the reader's convenience and to facilitate understanding. Thus, the titles or subtitles should not be construed so as to limit the scope of the disclosure, e.g., by grouping features which were presented in a particular order or together simply to facilitate understanding. Unless otherwise defined herein, all technical and scientific terms used herein have the same meaning as commonly understood by one of ordinary skill in the art to which this disclosure pertains. In the case of conflict, this document, including any definitions provided herein, will control. A recital of one or more synonyms herein does not exclude the use of other synonyms. The use of examples anywhere in this specification including examples of any term discussed herein is illustrative only and is not intended to further limit the scope and meaning of the disclosure or of any exemplified term.

Similarly, despite the particular presentation in the figures herein, one skilled in the art will appreciate that actual data structures used to store information may differ from what is shown. For example, the data structures may be organized in a different manner, may contain more or less information than shown, may be compressed and/or encrypted, etc. The drawings and disclosure may omit common or well-known details in order to avoid confusion. Similarly, the figures may depict a particular series of operations to facilitate understanding, which are simply exemplary of a wider class of such collection of operations. Accordingly, one will readily recognize that additional, alternative, or fewer operations may often be used to achieve the same purpose or effect depicted in some of the flow diagrams. For example, data may be encrypted, though not presented as such in the figures, items may be considered in different looping patterns (“for” loop, “while” loop, etc.), or sorted in a different manner, to achieve the same or similar effect, etc.

Reference herein to “an embodiment” or “one embodiment” means that at least one embodiment of the disclosure includes a particular feature, structure, or characteristic described in connection with the embodiment. Thus, the phrase “in one embodiment” in various places herein is not necessarily referring to the same embodiment in each of those various places. Separate or alternative embodiments may not be mutually exclusive of other embodiments. One will recognize that various modifications may be made without deviating from the scope of the embodiments.

What is claimed is:

1. A system, comprising:
one or more processors, coupled with memory, to:
receive a plurality of data streams of information of a plurality of medical procedures, wherein the information comprises:
case metadata of the plurality of medical procedures;
a timeline defined by phase data relating to a plurality of phases identified for each of the plurality of medical procedures and task data relating to a plurality of tasks identified within each of the plurality of phases; and
three-dimensional point cloud data for each of the plurality of medical procedures during at least portions of the plurality of phases and the plurality of tasks within each of the phases;
provide for display at least a portion of the information using a hierarchical user interface structure, wherein the hierarchical user interface structure comprises:
a first level of a user interface to display, based at least on the three-dimensional point cloud data, a three-dimensional point cloud representation of a task of a phase selected from the timeline of a second level of user interface; and
the second level of the user interface to display the timeline and a portion of the case metadata associated with the timeline.

2. The system of claim 1, wherein the first level of the user interface is displayed in response to a user selecting a user interactive element displayed in the second level of the user interface, the user interactive element corresponds to a portion of the timeline.

3. The system of claim 1, wherein the hierarchical user interface structure further comprises a third level of the user interface to display case data, wherein the case data comprises metrics and statistics determined based on the case metadata for at least one of:
a medical procedure of the plurality of medical procedures;
a phase of the plurality of phases;
a task of the plurality of tasks;
an operating room (OR);
a hospital;
a robotic system or instrument; or
medical staff.

4. The system of claim 3, wherein the second level of the user interface is displayed in response to a user selecting a user interactive element displayed in the third level of the user interface, the user interactive element corresponds to a portion of the case data, and the portion of the case data corresponds to the timeline displayed in the second level.

5. The system of claim 4, wherein one of:
the portion of the case data is for a medical procedure of the plurality of medical procedures, and the timeline displayed in the second level is a timeline of the medical procedure;
the portion of the case data is for an operating room (OR), and the timeline displayed in the second level is a timeline of at least one medical procedure performed in the OR;
the portion of the case data is for a hospital, and the timeline displayed in the second level is a timeline of at least one medical procedure performed in the hospital;
the portion of the case data is for a robotic system or instrument, and the timeline displayed in the second level is a timeline of at least one medical procedure performed using the robotic system or instrument; or
the portion of the case data is for a medical staff, and the timeline displayed in the second level is a timeline of at least one medical procedure performed by the medical staff.

6. The system of claim 3, wherein the hierarchical user interface structure further comprises a fourth level of the user interface to display aggregated data, wherein the aggregated data is determined based on or aggregated from the case data of at least one of:
two or more of the plurality of medical procedures;
two or more of the plurality of phases;
two or more of the plurality of tasks;
two or more a plurality of ORs;
two or more hospitals;
two or more robotic systems or instruments; or
two or more medical staff members.

7. The system of claim 6, wherein the third level of the user interface is displayed in response to a user selecting a user interactive element displayed in the fourth level of the user interface, the user interactive element corresponds to a portion of the aggregated data that corresponds to at least one of:
a medical procedure of the two or more of the plurality of medical procedures;
a phase of the two or more of the plurality of phases;
a task of the two or more of the plurality of tasks;
an OR of the two or more a plurality of ORs;
a hospital of the two or more hospitals;
a robotic system or instrument of the two or more robotic systems or instruments; or
a medical staff member of the two or more medical staff members.

8. The system of claim 6, wherein the hierarchical user interface structure further comprises a fifth level of the user interface to display highlights, wherein the highlights comprise a selected portion of the aggregated data.

9. The system of claim 8, wherein the fourth level of the user interface is displayed in response to a user selecting a user interactive element displayed in the fifth level of the user interface.

10. The system of claim 1, wherein the case metadata comprises at least one of identifying information of the plurality of medical procedures, identifying information of one or more operating rooms (ORs) in which the plurality of medical procedures are performed, identifying information of one or more hospitals in which the plurality of medical procedures are performed, identifying information of medical staff by which the plurality of medical procedures are performed, identifying information of one or more robotic systems or instruments used in the plurality of medical procedures, statistical information of the plurality of medical procedures, statistical information of the one or more ORs, statistical information of the one or more hospitals, statistical information of the medical staff, or statistical information of the one or more robotic systems or instruments.

11. The system of claim 10, wherein
the identifying information of the plurality of medical procedures comprises at least one of:
a name or type of each of the plurality of medical procedures;
a time at which or a time duration in which each of the plurality of medical procedures is performed; or
a modality of each of the plurality of medical procedures;
the identifying information of the one or more ORs comprises a name of each of the one or more ORs;
the identifying information of the one or more hospitals comprises a name of each of the one or more hospitals;
the identifying information of the medical staff comprises a name of each of one or more surgeons;
the identifying information of the one or more robotic systems or instruments comprises at least one of:
a name of each of the one or more robotic systems or instruments; or
an attribute of each of the one or more robotic systems or instruments;
the identifying information of at least one sensor comprises at least one of a name of each of the at least one sensor or a modality of each of the at least one sensor;
the statistical information of the plurality of medical procedures comprises a number of the plurality of medical procedures or a number of types of the plurality of medical procedures performed in the one or more hospital, in the one or more ORs, by the medical staff, or using the one or more robotic systems or instruments;
the statistical information of the one or more ORs comprises a number of the plurality of medical procedures or a number of types of the plurality of medical staff performed in each of the one or more ORs;
the statistical information of the one or more hospitals comprises a number of the plurality of medical procedures or a number of types of the plurality of medical staff performed in each of the one or more hospitals;
the statistical information of the medical staff comprises a number of the plurality of medical procedures or a number of types of the plurality of medical staff performed by the medical staff; and
the statistical information of the one or more robotic systems or instruments comprises a number of the plurality of medical procedures or a number of types of the plurality of medical staff performed by the one or more robotic systems or instruments.

12. The system of claim 1, wherein the one or more processors to filter out from displaying at least a portion of the information of the plurality of medical procedures based on at least one a volume of the plurality of medical procedures, time, a location, or a medical staff member.

13. The system of claim 1, wherein the one or more processors to:
display, in at least one of the first level of the user interface or the second level of the user interface, a plurality of options for filtering; and
in response to receiving user input corresponding to a first option of the plurality of options, display a portion of the information of the plurality of medical procedures corresponding to the first option.

14. The system of claim 1, wherein the one or more processors to:
display, in at least one of the first level of the user interface or the second level of the user interface, a plurality of options for filtering; and
in response to receiving user input corresponding to a first option of the plurality of options, sort remaining options of the plurality of options based on the first option.

15. The system of claim 13, wherein the plurality of options are sorted based on a case volume associated with each of the plurality of options, a time associated with each of the plurality of options, a location associated with each of the plurality of options, or a surgeon associated with each of the plurality of options.

16. The system of claim 1, wherein the one or more processors to display, in at least one level of the hierarchical user interface structure, at least one suggested view based on a hospital, for a plurality of hospitals, for an operating room (OR), for a user, for a medical staff member, or for a procedure.

17. The system of claim 1, wherein the one or more processors to display, in at least one level of the hierarchical user interface structure, a suggested view based on a hospital, the suggested view comprises at least one option to provide the information of the plurality of medical procedures for one or more of:
at least one operating room (OR) of the hospital;
at least one procedure performed in the hospital; or
or at least one medical staff member performing the at least one procedure in the hospital.

18. The system of claim 1, wherein the one or more processors to display, in at least one level of the hierarchical user interface structure, a suggested view based on an operating room (OR), the suggested view comprises at least one option to provide the information of the plurality of medical procedures for one or more of:
at least one procedure performed in the OR; or
at least one medical staff member performing the at least one procedure in the OR.

19. The system of claim 1, wherein the one or more processors to display, in at least one level of the hierarchical user interface structure, a suggested view based on a medical staff member, the suggested view comprises at least one option to provide the information of the plurality of medical procedures for one or more of:
at least one procedure performed by the medical staff member;
at least one type of the at least one procedure performed by the medical staff member; or
at least one classification of the at least one procedure performed by the medical staff member.

20. The system of claim 1, wherein the one or more processors to display, in at least one level of the hierarchical user interface structure, a suggested view based on a medical procedure of the plurality of medical procedures, the suggested view comprises at least one option to provide the information of the plurality of medical procedures for one or more of:
at least one operating room (OR) in which the medical procedure is performed;
at least one hospital in which the medical procedure is performed;
at least one medical staff member who has performed in the medical procedure;
at least one type of the medical procedure; or
at least one classification of the medical procedure.

21. The system of claim 1, wherein the information of the plurality of medical procedures comprises metrics values determined using the plurality of data streams, wherein the metric values are determined by:
determining a duration of each of one or more intervals based on the plurality of data streams; and
determining the metric values based on a number of medical staff members in a surgical theater during a nonoperative period and motion in the surgical theater during the nonoperative period; and
determining the metric values based on the determined duration of each of the one or more intervals.

22. The system of claim 21, wherein the metric values comprise one or more of:
a metric associated with temporal workflow;
a metric associated with scheduling; and
a metric associated with human resources.

23. The system of claim 22, wherein the metric values comprise one or more of:
an Efficiency metric value;
a Consistency metric value;
an Adverse Events metric value;
a Case Volume metric value;
a First Case Turnovers metric value;
a Delay metric value;
an OR Traffic metric value;
a Room Layout metric value; and
a Modality Conversion metric value.

24. The system of claim 21, wherein the metric values comprise a composite score value, the composite score value determined based upon one or more of:
an Efficiency metric value;
a Consistency metric value;
an Adverse Events metric value;
a Case Volume metric value;
a First Case Turnovers metric value;
a Delay metric value;
an OR Traffic metric value;
a Room Layout metric value; and
a Modality Conversion metric value.

25. The system of claim 24, wherein the one or more processors to cause the duration of each of the one or more intervals and the composite score value to be presented simultaneously in at least one level of the hierarchical user interface structure.

26. The system of claim 21, wherein determining the motion in the surgical theater during the nonoperative period comprises:
determining an optical flow of theater data of the plurality of data streams;
determining that the optical flow of the theater data corresponds to an object associated with a metric value determination; and
determining a standardized motion associated with the optical flow.

27. The system of claim 26, wherein the theater data comprises theater-wide visual image data, theater-wide depth data, or both theater-wide visual image and theater-wide depth data.

28. The system of claim 21, wherein the one or more processors to cause the durations of each of the one or more intervals and the metric values to be presented simultaneously in at least one level of the hierarchical user interface structure.

29. The system of claim 21, wherein the one or more processors to detect, using a machine learning system, a portion of theater-wide data of the plurality of data streams associated with the nonoperative period from a dataset comprising one or more operative periods.

30. The system of claim 1, wherein the one or more processors, coupled with memory, to select at least the portion of the information of the plurality of medical procedures according to a role of a user interacting with the user interface, wherein at least the portion of the information is mapped to the role of the user.

31. The system of claim 1, wherein
the role of the user comprises at least one of a surgeon, a medical staff member, a hospital administrator, a hospital group administrator, a cross-institution administrator, a consultant, or a student; and
at least one of:
the role of the surgeon or the medical staff member is mapped to at least the portion of the information of the plurality of medical procedures performed by a respective one of the surgeon or the medical staff member;
the role of the hospital administrator is mapped to at least the portion of the information of the plurality of medical procedures performed in a hospital; or
the role of the hospital group administrator, the cross-institution administrator, the consultant, or the student is mapped to at least the portion of the information of the plurality of medical procedures performed in a plurality of hospitals.

32. A non-transitory computer-readable medium comprising instructions configured to cause the one or more processors of the system of claim 1 to perform operations of the one or more processors in claim 1.

33. A method, comprising:
receiving a plurality of data streams of information of a plurality of medical procedures, wherein the information comprises:
case metadata of the plurality of medical procedures;
a timeline defined by phase data relating to a plurality of phases identified for each of the plurality of medical procedures and task data relating to a plurality of tasks identified within each of the plurality of phases; and
three-dimensional point cloud data for each of the plurality of medical procedures during at least portions of the plurality of phases and the plurality of tasks within each of the phases;
providing for display at least a portion of the information of the plurality of medical procedures using a hierarchical user interface structure, wherein the hierarchical user interface structure comprises:
a first level of a user interface to display, based at least on the three-dimensional point cloud data, a three-dimensional point cloud representation of a task of a phase selected from the timeline of a second level of user interface; and
the second level of the user interface to display the timeline and a portion of the case metadata associated with the timeline.

34. A system, comprising:
one or more processors, coupled with memory, to:
receive at least one data stream of information of a plurality of medical procedures; and
provide for display the information of the plurality of medical procedures according to a hierarchical graphical user interface (GUI) comprising a plurality of levels having a first level and a second level, wherein
a first GUI corresponds to the first level and is configured to be displayed suggested first information of the plurality of medical procedures in response to a user interactive element of a second GUI being selected, wherein the suggested first information of the plurality of medical procedures is determined based on at least one of a medical procedure, a medical staff member, or a location for performing the medical procedure, and
the second level corresponds to the second GUI displaying second information of the information of the plurality of medical procedures and a prompt to the first GUI to display the suggested first information of the information of the plurality of medical procedures.

35. The system of claim 34, wherein the first GUI is displayed in response to a user selecting a user interactive element displayed in the second GUI, the user interactive element corresponds to a portion of a timeline defined by phase data relating to a plurality of phases identified for each of the plurality of medical procedures and task data relating to a plurality of tasks identified within each of the plurality of phases.

36. The system of claim 34, wherein the hierarchical GUI further comprises a third level corresponding to a third GUI to display case data, wherein the case data comprises metrics and statistics determined based on case metadata for at least one of:
a medical procedure of the plurality of medical procedures;
a phase of the plurality of phases;
a task of the plurality of tasks;
an operating room (OR);
a hospital;
a robotic system or instrument; or
medical staff.

37. The system of claim 36, wherein the second GUI is displayed in response to a user selecting a user interactive element displayed in the third GUI, the user interactive element corresponds to a portion of the case data, and the portion of the case data corresponds to a timeline displayed in the second GUI, wherein the timeline is defined by phase data relating to a plurality of phases identified for each of the plurality of medical procedures and task data relating to a plurality of tasks identified within each of the plurality of phases.

38. The system of claim 37, wherein one of:
the portion of the case data is for a medical procedure of the plurality of medical procedures, and the timeline displayed in the second GUI is a timeline of the medical procedure;
the portion of the case data is for an OR, and the timeline displayed in the second GUI is a timeline of at least one medical procedure performed in the OR;
the portion of the case data is for a hospital, and the timeline displayed in the second GUI is a timeline of at least one medical procedure performed in the hospital;
the portion of the case data is for a robotic system or instrument, and the timeline displayed in the second GUI is a timeline of at least one medical procedure performed using the robotic system or instrument; or
the portion of the case data is for a medical staff, and the timeline displayed in the second GUI is a timeline of at least one medical procedure performed by the medical staff.

39. The system of claim 36, wherein the hierarchical GUI further comprises a fourth level corresponding to a fourth GUI to display aggregated data, wherein the aggregated data is determined based on or aggregated from the case data of at least one of:
two or more of the plurality of medical procedures;
two or more of the plurality of phases;
two or more of the plurality of tasks;
two or more a plurality of ORs;
two or more hospitals;
two or more robotic systems or instruments; or
two or more medical staff members.

40. The system of claim 39, wherein the third GUI is displayed in response to a user selecting a user interactive element displayed in the fourth GUI, the user interactive element corresponds to a portion of the aggregated data that corresponds to at least one of:
a medical procedure of the two or more of the plurality of medical procedures;
a phase of the two or more of the plurality of phases;
a task of the two or more of the plurality of tasks;
an OR of the two or more a plurality of ORs;
a hospital of the two or more hospitals;
a robotic system or instrument of the two or more robotic systems or instruments; or
a medical staff member of the two or more medical staff members.

41. The system of claim 39, wherein the hierarchical GUI further comprises a fifth level corresponding to a fifth GUI to display highlights, wherein the highlights comprise a selected portion of the aggregated data.

42. The system of claim 41, wherein the fourth GUI is displayed in response to a user selecting a user interactive element displayed in the fifth GUI.

43. The system of claim 34, wherein the suggested first information of the plurality of medical procedures is determined based on a hospital, for a plurality of hospitals, for an operating room (OR), for a user, for a medical staff member, or for a procedure.

44. The system of claim 34, wherein the suggested first information of the plurality of medical procedures is determined based on a hospital, the suggested first information of the plurality of medical procedures comprises one or more of:
at least one operating room (OR) of the hospital;
at least one procedure performed in the hospital; or
or at least one medical staff member performing the at least one procedure in the hospital.

45. The system of claim 34, wherein the suggested first information of the plurality of medical procedures is determined based on an operating room (OR), the suggested first information of the plurality of medical procedures comprises one or more of:
at least one procedure performed in the OR; or
at least one medical staff member performing the at least one procedure in the OR.

46. The system of claim 34, wherein the suggested first information of the plurality of medical procedures is determined based on the medical staff member, the suggested first information comprises one or more of:
at least one procedure performed by the medical staff member;
at least one type of the at least one procedure performed by the medical staff member; or
at least one classification of the at least one procedure performed by the medical staff member.

47. The system of claim 34, wherein the suggested first information is determined based on the medical procedure of the plurality of medical procedures, the suggested first information comprises one or more of:
at least one operating room (OR) in which the medical procedure is performed;
at least one hospital in which the medical procedure is performed;
at least one medical staff member who has performed in the medical procedure;
at least one type of the medical procedure; or
at least one classification of the medical procedure.

48. A non-transitory computer-readable medium comprising instructions configured to cause the one or more processors of the system of claim 34 to perform operations of the one or more processors in claim 34.

49. A method, comprising:
receiving at least one data stream of information of a plurality of medical procedures; and
providing for display the information of the plurality of medical procedures according to a hierarchical graphical user interface (GUI) comprising a plurality of levels having a first level and a second level, wherein
a first GUI corresponds to the first level and is configured to display suggested first information in response to a user interactive element of a second GUI being selected, wherein the suggested first information is determined based on at least one of a medical procedure, a medical staff member, a role of a user, or a location for performing the medical procedure, and
the second level corresponds to the second GUI displaying second information of the information of the plurality of medical procedures and a prompt to the first GUI to display the suggested first information of the information.
